SEQUENCE LISTING (1506 sequences)

SEQ ID NO: 1 - DNA, 20 bp, Homo sapeins
gccctcacacttctacaccg

SEQ ID NO: 2 - DNA, 22 bp, Homo sapeins
ccgactttatcacacacctacg

SEQ ID NO: 3 - DNA, 23 bp, Homo sapeins
cgacttccaaacaataataaacg

SEQ ID NO: 4 - DNA, 25 bp, Homo sapeins
ccctaataaatcaaaacatctaccg

SEQ ID NO: 5 - DNA, 21 bp, Homo sapeins
actttcaactacccctcaccg

SEQ ID NO: 6 - DNA, 21 bp, Homo sapeins
gtccaacccctaactctctcg

SEQ ID NO: 7 - DNA, 20 bp, Homo sapeins
acccgaatctctcctaaccg

SEQ ID NO: 8 - DNA, 21 bp, Homo sapeins
actctatacccgacgcctacg

SEQ ID NO: 9 - DNA, 20 bp, Homo sapeins
ctaacgccgaacctacttcg

SEQ ID NO: 10 - DNA, 23 bp, Homo sapeins
cgccaaatatcttttcttcttcg

SEQ ID NO: 11 - DNA, 21 bp, Homo sapeins
tatcccgacgtaacttcctcg

SEQ ID NO: 12 - DNA, 21 bp, Homo sapeins
ccgacctaacaaaacaactcg

SEQ ID NO: 13 - DNA, 23 bp, Homo sapeins
aaccttcttaccttcaaacctcg

SEQ ID NO: 14 - DNA, 21 bp, Homo sapeins
aaatactcccattctccaccg

SEQ ID NO: 15 - DNA, 21 bp, Homo sapeins
actaccactccccaaaaaacg

SEQ ID NO: 16 - DNA, 19 bp, Homo sapeins
cgacgaaaataccccaacg

SEQ ID NO: 17 - DNA, 19 bp, Homo sapeins
gcgctcaaccaattaaacg

SEQ ID NO: 18 - DNA, 21 bp, Homo sapeins
aattcccaacgtaacaacacg

SEQ ID NO: 19 - DNA, 21 bp, Homo sapeins
actcacccctcctttctaccg

SEQ ID NO: 20 - DNA, 19 bp, Homo sapeins
gcgtccgaattcctaaacg

SEQ ID NO: 21 - DNA, 20 bp, Homo sapeins
aacccgcgaaatttaaaacg

SEQ ID NO: 22 - DNA, 23 bp, Homo sapeins
caatacaaccaacattcctaacg

SEQ ID NO: 23 - DNA, 23 bp, Homo sapeins
caactcctaatccccaataaacg

SEQ ID NO: 24 - DNA, 22 bp, Homo sapeins
catctttaaacaaacttccccg

SEQ ID NO: 25 - DNA, 21 bp, Homo sapeins
aaacaaacaacctccctctcg

SEQ ID NO: 26 - DNA, 21 bp, Homo sapeins
ctttaatttctcccaaacccg

SEQ ID NO: 27 - DNA, 23 bp, Homo sapeins
aaaaatctctactaactcccccg

SEQ ID NO: 28 - DNA, 22 bp, Homo sapeins
aacaacacctctcacgcatacg

SEQ ID NO: 29 - DNA, 22 bp, Homo sapeins
caaaacaacgaaacaatacccg

SEQ ID NO: 30 - DNA, 21 bp, Homo sapeins
cgaaaacctaaccgctactcg

SEQ ID NO: 31 - DNA, 20 bp, Homo sapeins
ccccttccctcaactaaacg

SEQ ID NO: 32 - DNA, 23 bp, Homo sapeins
ctcctactccttctcctaatccg

SEQ ID NO: 33 - DNA, 22 bp, Homo sapeins
taaaacaacttctcccgaaacg

SEQ ID NO: 34 - DNA, 22 bp, Homo sapeins
tatctacgacaccaaatcgacg

SEQ ID NO: 35 - DNA, 21 bp, Homo sapeins
ccatttcctttacttcctccg

SEQ ID NO: 36 - DNA, 18 bp, Homo sapeins
cgccgcctacaaaaaacg

SEQ ID NO: 37 - DNA, 22 bp, Homo sapeins
cacgaaaaccaaaaaactaccg

SEQ ID NO: 38 - DNA, 21 bp, Homo sapeins
aataaccaatccaaaaccccg

SEQ ID NO: 39 - DNA, 23 bp, Homo sapeins
caaataaaattcttcctcttccg

SEQ ID NO: 40 - DNA, 25 bp, Homo sapeins
ctcgaatcaataacactactcaacg

SEQ ID NO: 41 - DNA, 23 bp, Homo sapeins
aaatattccccttacaaaaaccg

SEQ ID NO: 42 - DNA, 24 bp, Homo sapeins
caacctactattatatcgccaacg

SEQ ID NO: 43 - DNA, 20 bp, Homo sapeins
tacgtaaatacgcgaacccg

SEQ ID NO: 44 - DNA, 17 bp, Homo sapeins
caaacctacatcccgcg

SEQ ID NO: 45 - DNA, 22 bp, Homo sapeins
cgaaaaacctaaaaacgaaacg

SEQ ID NO: 46 - DNA, 23 bp, Homo sapeins
cgtacgcttcctaaataaaaccg

SEQ ID NO: 47 - DNA, 23 bp, Homo sapeins
tctttctctaataacccacaccg

SEQ ID NO: 48 - DNA, 20 bp, Homo sapeins
ccaacctaactctccttccg

SEQ ID NO: 49 - DNA, 22 bp, Homo sapeins
tcttaaatcttaacaatccccg

SEQ ID NO: 50 - DNA, 23 bp, Homo sapeins
cctaaatacctcctacgcaaacg

SEQ ID NO: 51 - DNA, 22 bp, Homo sapeins
atcaatcacgtaaaacgttccg

SEQ ID NO: 52 - DNA, 22 bp, Homo sapeins
acaccaacctctaacgataccg

SEQ ID NO: 53 - DNA, 23 bp, Homo sapeins
ctcaaaaccgtaattttcaaacg

SEQ ID NO: 54 - DNA, 23 bp, Homo sapeins
aaaactaacctctcctaccctcg

SEQ ID NO: 55 - DNA, 26 bp, Homo sapeins
cctaaaacaaataacacaataccacg

SEQ ID NO: 56 - DNA, 21 bp, Homo sapeins
ataaaaacgaaacccgactcg

SEQ ID NO: 57 - DNA, 22 bp, Homo sapeins
caaatcaataacgtcacatccg

SEQ ID NO: 58 - DNA, 21 bp, Homo sapeins
ctcaaccctaatacccatccg

SEQ ID NO: 59 - DNA, 21 bp, Homo sapeins
ccactccaatactcactcccg

SEQ ID NO: 60 - DNA, 22 bp, Homo sapeins
cgaacaaaacaaacaaaatccg

SEQ ID NO: 61 - DNA, 21 bp, Homo sapeins
cttaactaacgcgaaaccacg

SEQ ID NO: 62 - DNA, 23 bp, Homo sapeins
gaatctcactcctaaattcaacg

SEQ ID NO: 63 - DNA, 21 bp, Homo sapeins
gcgaacctacaaaacctaacg

SEQ ID NO: 64 - DNA, 23 bp, Homo sapeins
actcgataataataaacacgccg

SEQ ID NO: 65 - DNA, 22 bp, Homo sapeins
ccaataaaaccgacttaacccg

SEQ ID NO: 66 - DNA, 22 bp, Homo sapeins
cttccttaaactctctccaccg

SEQ ID NO: 67 - DNA, 22 bp, Homo sapeins
ataacaaccatcctacctcccg

SEQ ID NO: 68 - DNA, 22 bp, Homo sapeins
gaccgaccgtaaaaataaaacg

SEQ ID NO: 69 - DNA, 23 bp, Homo sapeins
cctctcccctaccctataaaacg

SEQ ID NO: 70 - DNA, 18 bp, Homo sapeins
aactacgcgacgactccg

SEQ ID NO: 71 - DNA, 23 bp, Homo sapeins
gaaaaacaccttccataaaaacg

SEQ ID NO: 72 - DNA, 21 bp, Homo sapeins
gcaataccataaacgtcaccg

SEQ ID NO: 73 - DNA, 22 bp, Homo sapeins
acgaattacacaatccaattcg

SEQ ID NO: 74 - DNA, 24 bp, Homo sapeins
aattaataaccaaccaaaaactcg

SEQ ID NO: 75 - DNA, 21 bp, Homo sapeins
tctctttccctttctcttccg

SEQ ID NO: 76 - DNA, 23 bp, Homo sapeins
aataaatttcacctctcaatccg

SEQ ID NO: 77 - DNA, 24 bp, Homo sapeins
aaaatcctttacccctaaattacg

SEQ ID NO: 78 - DNA, 20 bp, Homo sapeins
gcaccaacctaaactcaacg

SEQ ID NO: 79 - DNA, 22 bp, Homo sapeins
cccaactaaaactaaaaccccg

SEQ ID NO: 80 - DNA, 20 bp, Homo sapeins
gcctcctaataaaaaccccg

SEQ ID NO: 81 - DNA, 23 bp, Homo sapeins
tacctctctccaaatctctcacg

SEQ ID NO: 82 - DNA, 23 bp, Homo sapeins
tacgtatctctcaacctctttcg

SEQ ID NO: 83 - DNA, 23 bp, Homo sapeins
aatatcaaaacctctacgacccg

SEQ ID NO: 84 - DNA, 22 bp, Homo sapeins
gaacccaataaaatcaaaaccg

SEQ ID NO: 85 - DNA, 21 bp, Homo sapeins
cctcaaccaatcaacgatacg

SEQ ID NO: 86 - DNA, 22 bp, Homo sapeins
ttaaaaaccgtctaatcgaccg

SEQ ID NO: 87 - DNA, 20 bp, Homo sapeins
tccgttctataccacacccg

SEQ ID NO: 88 - DNA, 23 bp, Homo sapeins
tacttcattctctacacaacccg

SEQ ID NO: 89 - DNA, 20 bp, Homo sapeins
cctcctaatcctacgcgacg

SEQ ID NO: 90 - DNA, 20 bp, Homo sapeins
aacgctcgaaacgaactacg

SEQ ID NO: 91 - DNA, 21 bp, Homo sapeins
ccaactccgaactcataaacg

SEQ ID NO: 92 - DNA, 21 bp, Homo sapeins
actcctctacacctaccgccg

SEQ ID NO: 93 - DNA, 21 bp, Homo sapeins
accgatacgatccatatcccg

SEQ ID NO: 94 - DNA, 23 bp, Homo sapeins
aaaccaaaacttacaaaccaacg

SEQ ID NO: 95 - DNA, 23 bp, Homo sapeins
tttatccccaattaataattccg

SEQ ID NO: 96 - DNA, 20 bp, Homo sapeins
tttataactccgtctccgcg

SEQ ID NO: 97 - DNA, 21 bp, Homo sapeins
ctaccctatacgcctaacccg

SEQ ID NO: 98 - DNA, 18 bp, Homo sapeins
acgtaccgacgtccaacg

SEQ ID NO: 99 - DNA, 22 bp, Homo sapeins
cactcgaacacgtaacaaatcg

SEQ ID NO: 100 - DNA, 22 bp, Homo sapeins
ctttcctaattaacgccaaacg

SEQ ID NO: 101 - DNA, 22 bp, Homo sapeins
aaccgaaccaaaactaaatccg

SEQ ID NO: 102 - DNA, 23 bp, Homo sapeins
aataacccccaaaactaaactcg

SEQ ID NO: 103 - DNA, 23 bp, Homo sapeins
caaaaacaaatccaaatataccg

SEQ ID NO: 104 - DNA, 23 bp, Homo sapeins
caaccctaaacatctaaacaccg

SEQ ID NO: 105 - DNA, 18 bp, Homo sapeins
tcattccactccaacccg

SEQ ID NO: 106 - DNA, 24 bp, Homo sapeins
ctaaaaaatctccctcttatctcg

SEQ ID NO: 107 - DNA, 18 bp, Homo sapeins
aacccttccaccctaccg

SEQ ID NO: 108 - DNA, 24 bp, Homo sapeins
cacaaatcctaaaataccaaaacg

SEQ ID NO: 109 - DNA, 23 bp, Homo sapeins
aactacaataaaccaaactcgcg

SEQ ID NO: 110 - DNA, 21 bp, Homo sapeins
gtataaaatacgccaccctcg

SEQ ID NO: 111 - DNA, 20 bp, Homo sapeins
cccatcccctaataatcacg

SEQ ID NO: 112 - DNA, 26 bp, Homo sapeins
aacaataacaaacatctcttataccg

SEQ ID NO: 113 - DNA, 22 bp, Homo sapeins
actataacccaacccaactacg

SEQ ID NO: 114 - DNA, 25 bp, Homo sapeins
aacactaaaccataaaatctcaacg

SEQ ID NO: 115 - DNA, 22 bp, Homo sapeins
cctacaatctacgcaaataccg

SEQ ID NO: 116 - DNA, 26 bp, Homo sapeins
cctatattcgcacacaatactattcg

SEQ ID NO: 117 - DNA, 22 bp, Homo sapeins
accgacctaacgactctcaacg

SEQ ID NO: 118 - DNA, 20 bp, Homo sapeins
acaccacacacacacacacg

SEQ ID NO: 119 - DNA, 23 bp, Homo sapeins
aaccgattatctttataaccgcg

SEQ ID NO: 120 - DNA, 19 bp, Homo sapeins
cacaaaaaacaaaaacgcg

SEQ ID NO: 121 - DNA, 22 bp, Homo sapeins
cctcaccccaaataaaaactcg

SEQ ID NO: 122 - DNA, 22 bp, Homo sapeins
ctacacccaaatttccattacg

SEQ ID NO: 123 - DNA, 22 bp, Homo sapeins
aatcacgatccaacctctaccg

SEQ ID NO: 124 - DNA, 23 bp, Homo sapeins
atacccaaaaacaataaccaacg

SEQ ID NO: 125 - DNA, 23 bp, Homo sapeins
gaacgacgaaacaataacctacg

SEQ ID NO: 126 - DNA, 25 bp, Homo sapeins
tcatcaaaacatctataaatcgtcg

SEQ ID NO: 127 - DNA, 22 bp, Homo sapeins
tccaaaactacccaaacctacg

SEQ ID NO: 128 - DNA, 18 bp, Homo sapeins
gcgatccctaaacttccg

SEQ ID NO: 129 - DNA, 23 bp, Homo sapeins
cacaacttatccctacttacccg

SEQ ID NO: 130 - DNA, 23 bp, Homo sapeins
ttaacccaaacaataaccctacg

SEQ ID NO: 131 - DNA, 22 bp, Homo sapeins
ttctctcaaaccacttatcccg

SEQ ID NO: 132 - DNA, 22 bp, Homo sapeins
gcaaaaccaaaaactcctaccg

SEQ ID NO: 133 - DNA, 23 bp, Homo sapeins
cgatccctttaaatactcgtacg

SEQ ID NO: 134 - DNA, 22 bp, Homo sapeins
cccaaaaacactcaaatactcg

SEQ ID NO: 135 - DNA, 19 bp, Homo sapeins
acacaaacgaacccacacg

SEQ ID NO: 136 - DNA, 22 bp, Homo sapeins
aacaaacccaacctcttactcg

SEQ ID NO: 137 - DNA, 22 bp, Homo sapeins
gaaccgaaatctttataacgcg

SEQ ID NO: 138 - DNA, 22 bp, Homo sapeins
caaatcacctttacctcttccg

SEQ ID NO: 139 - DNA, 22 bp, Homo sapeins
ccgctaaaaacaaaactcatcg

SEQ ID NO: 140 - DNA, 21 bp, Homo sapeins
cgataaaccctacctccaacg

SEQ ID NO: 141 - DNA, 18 bp, Homo sapeins
ctacatcctcgccgaacg

SEQ ID NO: 142 - DNA, 21 bp, Homo sapeins
atactcgctaaacacaacgcg

SEQ ID NO: 143 - DNA, 26 bp, Homo sapeins
gacctataccctttataaaatacgcg

SEQ ID NO: 144 - DNA, 23 bp, Homo sapeins
tcttaacgctaacctcaataacg

SEQ ID NO: 145 - DNA, 23 bp, Homo sapeins
tacctaaccatcctcaaatttcg

SEQ ID NO: 146 - DNA, 23 bp, Homo sapeins
cttcctcgaaatctaacaattcg

SEQ ID NO: 147 - DNA, 22 bp, Homo sapeins
actccgaataaaactaccaccg

SEQ ID NO: 148 - DNA, 23 bp, Homo sapeins
cccacccaataatcaaataaacg

SEQ ID NO: 149 - DNA, 23 bp, Homo sapeins
acccataattaccataatttccg

SEQ ID NO: 150 - DNA, 21 bp, Homo sapeins
cacacccaacctaataccacg

SEQ ID NO: 151 - DNA, 23 bp, Homo sapeins
aaacaataaacgcgaactctacg

SEQ ID NO: 152 - DNA, 19 bp, Homo sapeins
aaacccaacataacccacg

SEQ ID NO: 153 - DNA, 17 bp, Homo sapeins
aaccctatccttccccg

SEQ ID NO: 154 - DNA, 21 bp, Homo sapeins
ccataaacacccaaatatccg

SEQ ID NO: 155 - DNA, 27 bp, Homo sapeins
tcaacatattaaaacatattcctaacg

SEQ ID NO: 156 - DNA, 24 bp, Homo sapeins
ccacattacttaaaaactcgaacg

SEQ ID NO: 157 - DNA, 22 bp, Homo sapeins
aatcaaataaccacacgacacg

SEQ ID NO: 158 - DNA, 23 bp, Homo sapeins
aacctaaccctttaaatcttccg

SEQ ID NO: 159 - DNA, 22 bp, Homo sapeins
ctttttcttaaccctacccccg

SEQ ID NO: 160 - DNA, 23 bp, Homo sapeins
tacccgtacaatactaaaatccg

SEQ ID NO: 161 - DNA, 24 bp, Homo sapeins
attattaatcaccaacaaacaacg

SEQ ID NO: 162 - DNA, 22 bp, Homo sapeins
tctcccttcattattcattccg

SEQ ID NO: 163 - DNA, 23 bp, Homo sapeins
tcttcctaaatactcataacgcg

SEQ ID NO: 164 - DNA, 21 bp, Homo sapeins
aacctaacccaacaaacaacg

SEQ ID NO: 165 - DNA, 21 bp, Homo sapeins
tctacaaatcccgaaatctcg

SEQ ID NO: 166 - DNA, 25 bp, Homo sapeins
cacaaaacccaactatataaaaacg

SEQ ID NO: 167 - DNA, 22 bp, Homo sapeins
tcactacaacctctacctcccg

SEQ ID NO: 168 - DNA, 21 bp, Homo sapeins
acgtaatcacgcaacatctcg

SEQ ID NO: 169 - DNA, 26 bp, Homo sapeins
tcaccatattaaccaaactaatctcg

SEQ ID NO: 170 - DNA, 23 bp, Homo sapeins
ttttaaccctaacctttaacccg

SEQ ID NO: 171 - DNA, 22 bp, Homo sapeins
cctaacttttaacctttccccg

SEQ ID NO: 172 - DNA, 22 bp, Homo sapeins
aaacgaaacttacttcgcaacg

SEQ ID NO: 173 - DNA, 22 bp, Homo sapeins
aaaccctaaaaactaaaccccg

SEQ ID NO: 174 - DNA, 25 bp, Homo sapeins
aataaaccctacaccttctatctcg

SEQ ID NO: 175 - DNA, 23 bp, Homo sapeins
aaaaaatacaaaacccactaccg

SEQ ID NO: 176 - DNA, 23 bp, Homo sapeins
acaacctaaaaattacccatccg

SEQ ID NO: 177 - DNA, 22 bp, Homo sapeins
cccttcattacgacgaactacg

SEQ ID NO: 178 - DNA, 22 bp, Homo sapeins
aacgactctcaaaccctatccg

SEQ ID NO: 179 - DNA, 18 bp, Homo sapeins
ctactcccaccgcattcg

SEQ ID NO: 180 - DNA, 22 bp, Homo sapeins
caatcatatttccaaaatcccg

SEQ ID NO: 181 - DNA, 25 bp, Homo sapeins
tcctaatcaccctctatcttctacg

SEQ ID NO: 182 - DNA, 22 bp, Homo sapeins
aaaaaatcaccctaaccaaacg

SEQ ID NO: 183 - DNA, 24 bp, Homo sapeins
aaataacctaatcacttcaaaccg

SEQ ID NO: 184 - DNA, 21 bp, Homo sapeins
ccccttacgacctttctaacg

SEQ ID NO: 185 - DNA, 23 bp, Homo sapeins
cacctcaatacctcgtactcacg

SEQ ID NO: 186 - DNA, 22 bp, Homo sapeins
cacataccgctcgtaatattcg

SEQ ID NO: 187 - DNA, 22 bp, Homo sapeins
taacactcaatcccccttaacg

SEQ ID NO: 188 - DNA, 22 bp, Homo sapeins
aaacaaattcaaacccaacacg

SEQ ID NO: 189 - DNA, 18 bp, Homo sapeins
acccgataaaacgcaacg

SEQ ID NO: 190 - DNA, 20 bp, Homo sapeins
aacgaccaaatacgccttcg

SEQ ID NO: 191 - DNA, 22 bp, Homo sapeins
cacgaaaatactaaacctcccg

SEQ ID NO: 192 - DNA, 21 bp, Homo sapeins
acgcaaataacttcaccctcg

SEQ ID NO: 193 - DNA, 22 bp, Homo sapeins
ccccttataactaaacgaaacg

SEQ ID NO: 194 - DNA, 18 bp, Homo sapeins
tcctacaaaacccccacg

SEQ ID NO: 195 - DNA, 21 bp, Homo sapeins
ataaccgccttcctatttccg

SEQ ID NO: 196 - DNA, 21 bp, Homo sapeins
ccgcctaaaacctaaaaaccg

SEQ ID NO: 197 - DNA, 22 bp, Homo sapeins
aaatcttaacaaacgaccgacg

SEQ ID NO: 198 - DNA, 20 bp, Homo sapeins
ctctcctcaacgctaatccg

SEQ ID NO: 199 - DNA, 23 bp, Homo sapeins
aaaataacaacctaacccctacg

SEQ ID NO: 200 - DNA, 19 bp, Homo sapeins
ccgaaacctaactccctcg

SEQ ID NO: 201 - DNA, 17 bp, Homo sapeins
aatcaacgccgacctcg

SEQ ID NO: 202 - DNA, 23 bp, Homo sapeins
cgctcaattacttaacaacctcg

SEQ ID NO: 203 - DNA, 18 bp, Homo sapeins
gactcatcgcaacctccg

SEQ ID NO: 204 - DNA, 17 bp, Homo sapeins
ctctaccccctcccccg

SEQ ID NO: 205 - DNA, 23 bp, Homo sapeins
aacgcctaaattacctacaaccg

SEQ ID NO: 206 - DNA, 23 bp, Homo sapeins
acttcactttctccctctcttcg

SEQ ID NO: 207 - DNA, 23 bp, Homo sapeins
tcaaacacataactttaaccccg

SEQ ID NO: 208 - DNA, 23 bp, Homo sapeins
tccccactcttattaactttccg

SEQ ID NO: 209 - DNA, 21 bp, Homo sapeins
cgccaactctataatcgaacg

SEQ ID NO: 210 - DNA, 24 bp, Homo sapeins
gaattctctaactcacttccttcg

SEQ ID NO: 211 - DNA, 23 bp, Homo sapeins
gctaactttttcaaacctttccg

SEQ ID NO: 212 - DNA, 22 bp, Homo sapeins
aacgtataaacgccacctctcg

SEQ ID NO: 213 - DNA, 22 bp, Homo sapeins
cccgaatcaactctaacactcg

SEQ ID NO: 214 - DNA, 19 bp, Homo sapeins
gccctaacttccaactccg

SEQ ID NO: 215 - DNA, 22 bp, Homo sapeins
tcattaataaaaccgaccgacg

SEQ ID NO: 216 - DNA, 22 bp, Homo sapeins
aaacctactaactccacccacg

SEQ ID NO: 217 - DNA, 22 bp, Homo sapeins
aacccaacctctatcgaaaacg

SEQ ID NO: 218 - DNA, 26 bp, Homo sapeins
tccctataacgtaaccttaaaaaacg

SEQ ID NO: 219 - DNA, 22 bp, Homo sapeins
gaatcacgaaatcaaaaattcg

SEQ ID NO: 220 - DNA, 23 bp, Homo sapeins
ataactcaactcacaacctctcg

SEQ ID NO: 221 - DNA, 21 bp, Homo sapeins
cttaacctacctcgacctccg

SEQ ID NO: 222 - DNA, 26 bp, Homo sapeins
accaacctaaaaactaaaaaataacg

SEQ ID NO: 223 - DNA, 25 bp, Homo sapeins
aaatcttactatatctcccaaaccg

SEQ ID NO: 224 - DNA, 24 bp, Homo sapeins
caaaaatcataccattacactccg

SEQ ID NO: 225 - DNA, 18 bp, Homo sapeins
attaccgacgatccaccg

SEQ ID NO: 226 - DNA, 22 bp, Homo sapeins
attaaaccaaccttctttcccg

SEQ ID NO: 227 - DNA, 19 bp, Homo sapeins
catccactccgtctctccg

SEQ ID NO: 228 - DNA, 18 bp, Homo sapeins
atcctccctcgctaaccg

SEQ ID NO: 229 - DNA, 19 bp, Homo sapeins
cgaacgtaataaaaccgcg

SEQ ID NO: 230 - DNA, 24 bp, Homo sapeins
cctaactaacacgaacatttctcg

SEQ ID NO: 231 - DNA, 24 bp, Homo sapeins
ccaatctctaatatccacctctcg

SEQ ID NO: 232 - DNA, 20 bp, Homo sapeins
caactcaaaacaaacgctcg

SEQ ID NO: 233 - DNA, 23 bp, Homo sapeins
ttcgttaaaatacctacccctcg

SEQ ID NO: 234 - DNA, 23 bp, Homo sapeins
ccttttcaattactcacaatccg

SEQ ID NO: 235 - DNA, 21 bp, Homo sapeins
gctttaaccaatcaacgaacg

SEQ ID NO: 236 - DNA, 25 bp, Homo sapeins
catttctatacaaaccctacttccg

SEQ ID NO: 237 - DNA, 22 bp, Homo sapeins
aaaattcgacctcaatcaaacg

SEQ ID NO: 238 - DNA, 22 bp, Homo sapeins
aataaacgttctaaccctcgcg

SEQ ID NO: 239 - DNA, 20 bp, Homo sapeins
tcccgctcaatcaaactacg

SEQ ID NO: 240 - DNA, 24 bp, Homo sapeins
cactacgatattactccaaatccg

SEQ ID NO: 241 - DNA, 22 bp, Homo sapeins
acaaaaactcccttaaaccccg

SEQ ID NO: 242 - DNA, 23 bp, Homo sapeins
cgaatccgacattaataaaaacg

SEQ ID NO: 243 - DNA, 21 bp, Homo sapeins
cctaacttaaatccccctccg

SEQ ID NO: 244 - DNA, 23 bp, Homo sapeins
acgccctatataaaacgactacg

SEQ ID NO: 245 - DNA, 21 bp, Homo sapeins
caaaaccaatcaaaacgaacg

SEQ ID NO: 246 - DNA, 23 bp, Homo sapeins
ccaactttaacttcttcaaaacg

SEQ ID NO: 247 - DNA, 22 bp, Homo sapeins
tctccgctctaaaacgataacg

SEQ ID NO: 248 - DNA, 24 bp, Homo sapeins
aaaaattattttctccacatttcg

SEQ ID NO: 249 - DNA, 22 bp, Homo sapeins
acatcattttaccaatttcgcg

SEQ ID NO: 250 - DNA, 19 bp, Homo sapeins
aatcattcccgaccactcg

SEQ ID NO: 251 - DNA, 22 bp, Homo sapeins
ccaacaccgctataatataccg

SEQ ID NO: 252 - DNA, 22 bp, Homo sapeins
aaccccattttctatcgaaacg

SEQ ID NO: 253 - DNA, 22 bp, Homo sapeins
aatataaacgatcacgaccccg

SEQ ID NO: 254 - DNA, 21 bp, Homo sapeins
tacgctttcctaaaattcccg

SEQ ID NO: 255 - DNA, 23 bp, Homo sapeins
aacgtccctcaattaaataaccg

SEQ ID NO: 256 - DNA, 21 bp, Homo sapeins
cctctcttctaaaccctcccg

SEQ ID NO: 257 - DNA, 21 bp, Homo sapeins
ccgaaaaataaccgaaaaacg

SEQ ID NO: 258 - DNA, 20 bp, Homo sapeins
gccgaacaaaacgaactacg

SEQ ID NO: 259 - DNA, 23 bp, Homo sapeins
gatctcgactcactacaacctcg

SEQ ID NO: 260 - DNA, 22 bp, Homo sapeins
cgcgctactacactcctaaacg

SEQ ID NO: 261 - DNA, 20 bp, Homo sapeins
ccgatttaaaaaccaacccg

SEQ ID NO: 262 - DNA, 21 bp, Homo sapeins
cgatacgaaacctacgaaccg

SEQ ID NO: 263 - DNA, 21 bp, Homo sapeins
gcgataacatcaaccgatacg

SEQ ID NO: 264 - DNA, 22 bp, Homo sapeins
aacgcttcttcaaattcctacg

SEQ ID NO: 265 - DNA, 22 bp, Homo sapeins
caacaatcgaataaaccgatcg

SEQ ID NO: 266 - DNA, 23 bp, Homo sapeins
tactactcccgctaattcctacg

SEQ ID NO: 267 - DNA, 22 bp, Homo sapeins
aaaaaatcaatcaaattttccg

SEQ ID NO: 268 - DNA, 23 bp, Homo sapeins
cctcctacgactccataactacg

SEQ ID NO: 269 - DNA, 23 bp, Homo sapeins
ctaacattacaaacctcgcttcg

SEQ ID NO: 270 - DNA, 23 bp, Homo sapeins
cttccacatctaaactacttccg

SEQ ID NO: 271 - DNA, 23 bp, Homo sapeins
tactactacttctaccgctcccg

SEQ ID NO: 272 - DNA, 18 bp, Homo sapeins
tcaacgaccgtacccacg

SEQ ID NO: 273 - DNA, 23 bp, Homo sapeins
acttaaacctcaaacaaatcacg

SEQ ID NO: 274 - DNA, 20 bp, Homo sapeins
ttccaacaaatttcctaccg

SEQ ID NO: 275 - DNA, 22 bp, Homo sapeins
acattccttttcgtcaaaatcg

SEQ ID NO: 276 - DNA, 24 bp, Homo sapeins
cattaccaacaacttttaaatccg

SEQ ID NO: 277 - DNA, 20 bp, Homo sapeins
acaaactccgcctaacaccg

SEQ ID NO: 278 - DNA, 22 bp, Homo sapeins
ccaaacgcctataccttaaccg

SEQ ID NO: 279 - DNA, 22 bp, Homo sapeins
taaaacgaccaaaacacaaacg

SEQ ID NO: 280 - DNA, 22 bp, Homo sapeins
tcaaaaactaacaaaccaaacg

SEQ ID NO: 281 - DNA, 17 bp, Homo sapeins
atcccctcaacccaacg

SEQ ID NO: 282 - DNA, 23 bp, Homo sapeins
ccttaacgcgtatcctaatctcg

SEQ ID NO: 283 - DNA, 22 bp, Homo sapeins
aatccgcattaaaccaaatacg

SEQ ID NO: 284 - DNA, 23 bp, Homo sapeins
gaaaaccgaaacttaaatcctcg

SEQ ID NO: 285 - DNA, 17 bp, Homo sapeins
aaaacccgaaaaccccg

SEQ ID NO: 286 - DNA, 23 bp, Homo sapeins
cctatcgacctctaatacactcg

SEQ ID NO: 287 - DNA, 23 bp, Homo sapeins
caaacacaactcgatttaaaacg

SEQ ID NO: 288 - DNA, 19 bp, Homo sapeins
cctaaatccgtctttcgcg

SEQ ID NO: 289 - DNA, 23 bp, Homo sapeins
cccattctatctaccctatctcg

SEQ ID NO: 290 - DNA, 21 bp, Homo sapeins
taaaaaacgcctaaacccacg

SEQ ID NO: 291 - DNA, 21 bp, Homo sapeins
aacgaaaataacgttttcccg

SEQ ID NO: 292 - DNA, 22 bp, Homo sapeins
gcaaccaaaacaaactacgacg

SEQ ID NO: 293 - DNA, 25 bp, Homo sapeins
tcatcttacaatcacataaccatcg

SEQ ID NO: 294 - DNA, 23 bp, Homo sapeins
gctaactccgaaacaactaaacg

SEQ ID NO: 295 - DNA, 22 bp, Homo sapeins
ctctaccccaaccacaactacg

SEQ ID NO: 296 - DNA, 22 bp, Homo sapeins
cccaacaaacttttccttttcg

SEQ ID NO: 297 - DNA, 22 bp, Homo sapeins
gcgattaatatcctaccctccg

SEQ ID NO: 298 - DNA, 19 bp, Homo sapeins
cccgatacaaaccacaacg

SEQ ID NO: 299 - DNA, 22 bp, Homo sapeins
gaaacgctactcctaactcacg

SEQ ID NO: 300 - DNA, 22 bp, Homo sapeins
gtaaaccaaaaacaacgtcccg

SEQ ID NO: 301 - DNA, 18 bp, Homo sapeins
aacgcaaacgaaacaccg

SEQ ID NO: 302 - DNA, 24 bp, Homo sapeins
accctataaacctaatcgttaacg

SEQ ID NO: 303 - DNA, 20 bp, Homo sapeins
gtctaccctccacatctccg

SEQ ID NO: 304 - DNA, 23 bp, Homo sapeins
aacgctcgataaacgataatacg

SEQ ID NO: 305 - DNA, 22 bp, Homo sapeins
acccctaactacttcaacctcg

SEQ ID NO: 306 - DNA, 22 bp, Homo sapeins
caaatcttaaaccctaatcccg

SEQ ID NO: 307 - DNA, 22 bp, Homo sapeins
cccttctatataaacctccccg

SEQ ID NO: 308 - DNA, 19 bp, Homo sapeins
aaacaactatccccacccg

SEQ ID NO: 309 - DNA, 23 bp, Homo sapeins
ccttactaccacacatttaaccg

SEQ ID NO: 310 - DNA, 23 bp, Homo sapeins
tcaaatcttatcaaacaccctcg

SEQ ID NO: 311 - DNA, 23 bp, Homo sapeins
aaacccaacctatatccaaatcg

SEQ ID NO: 312 - DNA, 22 bp, Homo sapeins
aatactaaaaacgcccctctcg

SEQ ID NO: 313 - DNA, 22 bp, Homo sapeins
ctttaaccgctaacctaatccg

SEQ ID NO: 314 - DNA, 21 bp, Homo sapeins
cccgaaacaactacgctatcg

SEQ ID NO: 315 - DNA, 22 bp, Homo sapeins
aattaccccacaacctaaaccg

SEQ ID NO: 316 - DNA, 18 bp, Homo sapeins
cgcaaatcccgcttatcg

SEQ ID NO: 317 - DNA, 20 bp, Homo sapeins
gccaaacaaaacaaaaaccg

SEQ ID NO: 318 - DNA, 23 bp, Homo sapeins
gcaacgcattacaaacaaatacg

SEQ ID NO: 319 - DNA, 25 bp, Homo sapeins
cactctctcgcttttataaaaaacg

SEQ ID NO: 320 - DNA, 23 bp, Homo sapeins
attacacaaaccgttctaacccg

SEQ ID NO: 321 - DNA, 22 bp, Homo sapeins
gaaaaactatccgccttacacg

SEQ ID NO: 322 - DNA, 21 bp, Homo sapeins
tcactcttatccccacctacg

SEQ ID NO: 323 - DNA, 21 bp, Homo sapeins
ccaccaacatcctaaataccg

SEQ ID NO: 324 - DNA, 17 bp, Homo sapeins
acacaacccaaaccgcg

SEQ ID NO: 325 - DNA, 19 bp, Homo sapeins
acctcaaatcctccaaccg

SEQ ID NO: 326 - DNA, 22 bp, Homo sapeins
cccaaatacaactacccaaacg

SEQ ID NO: 327 - DNA, 22 bp, Homo sapeins
aaccgactcgaatctacctacg

SEQ ID NO: 328 - DNA, 22 bp, Homo sapeins
actctaacgacccatcctaacg

SEQ ID NO: 329 - DNA, 23 bp, Homo sapeins
cttacatcctctaaatttccccg

SEQ ID NO: 330 - DNA, 22 bp, Homo sapeins
acaacttaatccccctaacccg

SEQ ID NO: 331 - DNA, 23 bp, Homo sapeins
ataacgaccgatcctactctccg

SEQ ID NO: 332 - DNA, 21 bp, Homo sapeins
ctaaaaacccttaaccctccg

SEQ ID NO: 333 - DNA, 23 bp, Homo sapeins
actattctctcctatttccctcg

SEQ ID NO: 334 - DNA, 23 bp, Homo sapeins
taaaactttctccccgaaaaacg

SEQ ID NO: 335 - DNA, 23 bp, Homo sapeins
aattcctccacttaaactacccg

SEQ ID NO: 336 - DNA, 22 bp, Homo sapeins
aactcaaaaccctccaaaatcg

SEQ ID NO: 337 - DNA, 23 bp, Homo sapeins
ctaactaaactcaacaacctccg

SEQ ID NO: 338 - DNA, 21 bp, Homo sapeins
cttaactaacgcgaaaccacg

SEQ ID NO: 339 - DNA, 23 bp, Homo sapeins
gaatctcactcctaaattcaacg

SEQ ID NO: 340 - DNA, 21 bp, Homo sapeins
aaaacctctacccgaaaaccg

SEQ ID NO: 341 - DNA, 22 bp, Homo sapeins
aatacgaactacttcacccccg

SEQ ID NO: 342 - DNA, 22 bp, Homo sapeins
caacccaaaacctactcctacg

SEQ ID NO: 343 - DNA, 22 bp, Homo sapeins
aaatttaaattaccctcctccg

SEQ ID NO: 344 - DNA, 23 bp, Homo sapeins
cccctctaattatctctttaccg

SEQ ID NO: 345 - DNA, 23 bp, Homo sapeins
caacctatccaaatcacaaaacg

SEQ ID NO: 346 - DNA, 21 bp, Homo sapeins
caaatctcccgaataaaatcg

SEQ ID NO: 347 - DNA, 17 bp, Homo sapeins
caaacactcccctcccg

SEQ ID NO: 348 - DNA, 22 bp, Homo sapeins
aaacctcaatctaaaaaccacg

SEQ ID NO: 349 - DNA, 18 bp, Homo sapeins
cccctccccaaataatcg

SEQ ID NO: 350 - DNA, 28 bp, Homo sapeins
catattatatcctaccatttaatacccg

SEQ ID NO: 351 - DNA, 22 bp, Homo sapeins
aaactcaacctcacaaatcacg

SEQ ID NO: 352 - DNA, 25 bp, Homo sapeins
ccaaaaccaataactttacaaaacg

SEQ ID NO: 353 - DNA, 21 bp, Homo sapeins
aaataaaataccacctccgcg

SEQ ID NO: 354 - DNA, 19 bp, Homo sapeins
aaaaaccgcgaaaatttcg

SEQ ID NO: 355 - DNA, 24 bp, Homo sapeins
aaaacacctaaacttaaaacctcg

SEQ ID NO: 356 - DNA, 19 bp, Homo sapeins
aaaccaacccaaatttccg

SEQ ID NO: 357 - DNA, 22 bp, Homo sapeins
accacatacacactacacaccg

SEQ ID NO: 358 - DNA, 22 bp, Homo sapeins
acacacacaaaccacatacacg

SEQ ID NO: 359 - DNA, 18 bp, Homo sapeins
aaacaaacaccaaccccg

SEQ ID NO: 360 - DNA, 23 bp, Homo sapeins
acatacgtacgacaatcctctcg

SEQ ID NO: 361 - DNA, 23 bp, Homo sapeins
aactcctaaactcaaacaatccg

SEQ ID NO: 362 - DNA, 21 bp, Homo sapeins
gatactcgaaaaccgaaaccg

SEQ ID NO: 363 - DNA, 25 bp, Homo sapeins
cacactccctactaaacctaaatcg

SEQ ID NO: 364 - DNA, 23 bp, Homo sapeins
ccaacctcaactcacaaaatacg

SEQ ID NO: 365 - DNA, 23 bp, Homo sapeins
taacatcacgacccaaataaccg

SEQ ID NO: 366 - DNA, 19 bp, Homo sapeins
acctcgcccctaaactacg

SEQ ID NO: 367 - DNA, 23 bp, Homo sapeins
taccaaccgactaaactaaaccg

SEQ ID NO: 368 - DNA, 19 bp, Homo sapeins
aataaccaaacgaacgccg

SEQ ID NO: 369 - DNA, 22 bp, Homo sapeins
caaaacaaaaacgaaaacgacg

SEQ ID NO: 370 - DNA, 24 bp, Homo sapeins
ccaaaccaacttatctatcctacg

SEQ ID NO: 371 - DNA, 22 bp, Homo sapeins
caaacctaaaattcctctttcg

SEQ ID NO: 372 - DNA, 22 bp, Homo sapeins
aacaaaaactacaactctcccg

SEQ ID NO: 373 - DNA, 23 bp, Homo sapeins
ccaatacactccaaacctaaacg

SEQ ID NO: 374 - DNA, 23 bp, Homo sapeins
aaaattacaaacgtaaaccaccg

SEQ ID NO: 375 - DNA, 22 bp, Homo sapeins
tcaaaccctctaacctcaaacg

SEQ ID NO: 376 - DNA, 23 bp, Homo sapiens
gtccccaaaactaaaaaacatcc

SEQ ID NO: 377 - DNA, 20 bp, Homo sapiens
cgccaaactaaaatcgaacc

SEQ ID NO: 378 - DNA, 21 bp, Homo sapiens
atccctacgcgaaaacgtaac

SEQ ID NO: 379 - DNA, 23 bp, Homo sapiens
ctcccctcaactttcttcaaatt

SEQ ID NO: 380 - DNA, 22 bp, Homo sapiens
cctcccacaccactcaaaaatt

SEQ ID NO: 381 - DNA, 26 bp, Homo sapiens
aactaccaatcatatttcctaaacca

SEQ ID NO: 382 - DNA, 21 bp, Homo sapiens
cgccactaacgctaaactcct

SEQ ID NO: 383 - DNA, 21 bp, Homo sapiens
ttcccccgaactccctactat

SEQ ID NO: 384 - DNA, 20 bp, Homo sapiens
ccctacgtaaacgacctcgc

SEQ ID NO: 385 - DNA, 21 bp, Homo sapiens
aatctttacccgaacgcttcc

SEQ ID NO: 386 - DNA, 23 bp, Homo sapiens
ccctctaaaaacgtacaaaccaa

SEQ ID NO: 387 - DNA, 19 bp, Homo sapiens
aaataaataaaccgcgccg

SEQ ID NO: 388 - DNA, 19 bp, Homo sapiens
ctccaacaccaatccgaca

SEQ ID NO: 389 - DNA, 26 bp, Homo sapiens
ttatcacacacttatttctcacttcc

SEQ ID NO: 390 - DNA, 17 bp, Homo sapiens
cctcctcgtctcctccc

SEQ ID NO: 391 - DNA, 20 bp, Homo sapiens
gtaaacgaaacaacgacccg

SEQ ID NO: 392 - DNA, 21 bp, Homo sapiens
gactattccgtaaacgccacc

SEQ ID NO: 393 - DNA, 24 bp, Homo sapiens
aactaaactacaactcacccaacc

SEQ ID NO: 394 - DNA, 24 bp, Homo sapiens
tccttcctttccttaccctacttt

SEQ ID NO: 395 - DNA, 21 bp, Homo sapiens
aaaccgaacctcgcttaaacc

SEQ ID NO: 396 - DNA, 20 bp, Homo sapiens
atccaaacaaaccgcaaact

SEQ ID NO: 397 - DNA, 22 bp, Homo sapiens
ctccctaacccaatctctaacg

SEQ ID NO: 398 - DNA, 26 bp, Homo sapiens
aacaaaaaatttctattccttcaacc

SEQ ID NO: 399 - DNA, 17 bp, Homo sapiens
cctcgtaacgcgtcgac

SEQ ID NO: 400 - DNA, 25 bp, Homo sapiens
cctaacccaactctaaaacaaacaa

SEQ ID NO: 401 - DNA, 23 bp, Homo sapiens
caacccgaaactattacaaaccg

SEQ ID NO: 402 - DNA, 22 bp, Homo sapiens
ctcgatccacaaactctccact

SEQ ID NO: 403 - DNA, 23 bp, Homo sapiens
attataatcttcccacctcccac

SEQ ID NO: 404 - DNA, 18 bp, Homo sapiens
caaacgaaacgcaacacc

SEQ ID NO: 405 - DNA, 23 bp, Homo sapiens
aaaaaacctacgaactcaaaccc

SEQ ID NO: 406 - DNA, 23 bp, Homo sapiens
aaacatcaaccctacgacctaaa

SEQ ID NO: 407 - DNA, 20 bp, Homo sapiens
acgaaccgacctaaactccc

SEQ ID NO: 408 - DNA, 22 bp, Homo sapiens
aaaacgctaattcctcgactcc

SEQ ID NO: 409 - DNA, 24 bp, Homo sapiens
aaatcctaactctacaccctcctc

SEQ ID NO: 410 - DNA, 28 bp, Homo sapiens
caaacgaattacttacccttacttatca

SEQ ID NO: 411 - DNA, 28 bp, Homo sapiens
aacaaatcttacacctcttctacatctc

SEQ ID NO: 412 - DNA, 24 bp, Homo sapiens
taaacaacaacctctcaaaatacg

SEQ ID NO: 413 - DNA, 24 bp, Homo sapiens
aaaacgcttaactctttctatccc

SEQ ID NO: 414 - DNA, 22 bp, Homo sapiens
ctctttccttttacgtcatccg

SEQ ID NO: 415 - DNA, 23 bp, Homo sapiens
acccatctcaactttcatcatca

SEQ ID NO: 416 - DNA, 24 bp, Homo sapiens
ccctacatttccctctacactaaa

SEQ ID NO: 417 - DNA, 23 bp, Homo sapiens
atatcacctcatccgactccctt

SEQ ID NO: 418 - DNA, 20 bp, Homo sapiens
actcctaaattccatccccg

SEQ ID NO: 419 - DNA, 24 bp, Homo sapiens
aacatccctaccctctctatacaa

SEQ ID NO: 420 - DNA, 21 bp, Homo sapiens
ccttctctaaatccgcgaaat

SEQ ID NO: 421 - DNA, 22 bp, Homo sapiens
aaacgacttcaaaaccccctac

SEQ ID NO: 422 - DNA, 17 bp, Homo sapiens
ccgcaaaaccacaacgc

SEQ ID NO: 423 - DNA, 23 bp, Homo sapiens
tctaaatccccctcctctaatcg

SEQ ID NO: 424 - DNA, 24 bp, Homo sapiens
acactactccccgctttatttatt

SEQ ID NO: 425 - DNA, 22 bp, Homo sapiens
aattatcctcctacgccgacct

SEQ ID NO: 426 - DNA, 23 bp, Homo sapiens
taaccaatcaccctcttcctttc

SEQ ID NO: 427 - DNA, 23 bp, Homo sapiens
ataattttcctccttcttccctc

SEQ ID NO: 428 - DNA, 19 bp, Homo sapiens
ttttcgccttacgatcacc

SEQ ID NO: 429 - DNA, 21 bp, Homo sapiens
ccacgctacgccaacacttat

SEQ ID NO: 430 - DNA, 23 bp, Homo sapiens
aacccgatctatccctataacga

SEQ ID NO: 431 - DNA, 27 bp, Homo sapiens
aaactattacatttactctccacctcc

SEQ ID NO: 432 - DNA, 23 bp, Homo sapiens
acacccgaatatttcctaacgaa

SEQ ID NO: 433 - DNA, 18 bp, Homo sapiens
ccaaccattccaccctca

SEQ ID NO: 434 - DNA, 22 bp, Homo sapiens
aacccaaccccctcttcattat

SEQ ID NO: 435 - DNA, 19 bp, Homo sapiens
aacgataaccgcacaatcc

SEQ ID NO: 436 - DNA, 23 bp, Homo sapiens
ccaccaaacatacaattccaact

SEQ ID NO: 437 - DNA, 23 bp, Homo sapiens
attctatccctatcctaatcgcc

SEQ ID NO: 438 - DNA, 22 bp, Homo sapiens
actcaaacttctctccgaaacg

SEQ ID NO: 439 - DNA, 21 bp, Homo sapiens
ccactttacaaaacgtcaccg

SEQ ID NO: 440 - DNA, 20 bp, Homo sapiens
gaacaaacgaaaattcccga

SEQ ID NO: 441 - DNA, 25 bp, Homo sapiens
tacccactaaaaactacaacttccc

SEQ ID NO: 442 - DNA, 20 bp, Homo sapiens
taaaacgacgtctcctcccc

SEQ ID NO: 443 - DNA, 22 bp, Homo sapiens
taataacatcaccccgaaatcg

SEQ ID NO: 444 - DNA, 23 bp, Homo sapiens
atatacaaactccgaaatcgcaa

SEQ ID NO: 445 - DNA, 22 bp, Homo sapiens
aaactccaaaacgcccctctac

SEQ ID NO: 446 - DNA, 25 bp, Homo sapiens
aaataaattcctcacacctcacaac

SEQ ID NO: 447 - DNA, 23 bp, Homo sapiens
cactacccaatcgtcgtatcaaa

SEQ ID NO: 448 - DNA, 26 bp, Homo sapiens
tattattaaaacctcaatttcccaaa

SEQ ID NO: 449 - DNA, 21 bp, Homo sapiens
cacccctttccactactcctt

SEQ ID NO: 450 - DNA, 27 bp, Homo sapiens
ttcatcctattaaaaataccacaaatt

SEQ ID NO: 451 - DNA, 21 bp, Homo sapiens
cctaattttaaaccctcgccc

SEQ ID NO: 452 - DNA, 23 bp, Homo sapiens
caccctcatacttccaaaaccta

SEQ ID NO: 453 - DNA, 23 bp, Homo sapiens
cttcctaatcctaccctacccta

SEQ ID NO: 454 - DNA, 20 bp, Homo sapiens
cccgctacgaaatctctcct

SEQ ID NO: 455 - DNA, 21 bp, Homo sapiens
cttaaaacacaaaccgctccc

SEQ ID NO: 456 - DNA, 24 bp, Homo sapiens
cctaatttctacaaccgctctacc

SEQ ID NO: 457 - DNA, 22 bp, Homo sapiens
tccctctttcacgatctcactc

SEQ ID NO: 458 - DNA, 21 bp, Homo sapiens
ttatctcgaactcgcccactt

SEQ ID NO: 459 - DNA, 23 bp, Homo sapiens
acaaatcccataacccacctaaa

SEQ ID NO: 460 - DNA, 23 bp, Homo sapiens
aaaacgatacctccgaaactcac

SEQ ID NO: 461 - DNA, 23 bp, Homo sapiens
gccttctctaaaccaacaacaca

SEQ ID NO: 462 - DNA, 22 bp, Homo sapiens
aactcctacgtttccccctaac

SEQ ID NO: 463 - DNA, 23 bp, Homo sapiens
ccgatttcctatacgtaacgtca

SEQ ID NO: 464 - DNA, 19 bp, Homo sapiens
cgctaactacgaccgcctc

SEQ ID NO: 465 - DNA, 22 bp, Homo sapiens
gacgaaaactccgaaaaacgac

SEQ ID NO: 466 - DNA, 23 bp, Homo sapiens
gatcaacaaaacgaaccaaaacg

SEQ ID NO: 467 - DNA, 19 bp, Homo sapiens
ctaaaccgattccgcgaac

SEQ ID NO: 468 - DNA, 23 bp, Homo sapiens
acaaccccaccttctctacctac

SEQ ID NO: 469 - DNA, 23 bp, Homo sapiens
aaaatcccgaaatcttacattcc

SEQ ID NO: 470 - DNA, 23 bp, Homo sapiens
aaattcctcaattattcctccgc

SEQ ID NO: 471 - DNA, 22 bp, Homo sapiens
aacaactcgtccgaataaccaa

SEQ ID NO: 472 - DNA, 23 bp, Homo sapiens
ataacccttcgaccgatacaaac

SEQ ID NO: 473 - DNA, 23 bp, Homo sapiens
aaatacgcaaactacctcaaacc

SEQ ID NO: 474 - DNA, 19 bp, Homo sapiens
ttacacgcccgcgaactat

SEQ ID NO: 475 - DNA, 25 bp, Homo sapiens
aatccaccaatcaataaacaacttc

SEQ ID NO: 476 - DNA, 23 bp, Homo sapiens
ccaaacctccacaaatcttcctt

SEQ ID NO: 477 - DNA, 22 bp, Homo sapiens
ccaccaaaacccaactctaaaa

SEQ ID NO: 478 - DNA, 25 bp, Homo sapiens
tattcaaatacccaaaactaaaccc

SEQ ID NO: 479 - DNA, 28 bp, Homo sapiens
cacacttaaatctacctctattacctcc

SEQ ID NO: 480 - DNA, 20 bp, Homo sapiens
atcctaacccctacaacccc

SEQ ID NO: 481 - DNA, 29 bp, Homo sapiens
aaaatacctatccctaaataaaaatcatt

SEQ ID NO: 482 - DNA, 21 bp, Homo sapiens
ccttccttctaacccgctaaa

SEQ ID NO: 483 - DNA, 26 bp, Homo sapiens
aaacgtactttcttaaaccttaaacg

SEQ ID NO: 484 - DNA, 20 bp, Homo sapiens
cactacactccaacccgaac

SEQ ID NO: 485 - DNA, 23 bp, Homo sapiens
accttattacgacgtcgacacat

SEQ ID NO: 486 - DNA, 25 bp, Homo sapiens
attacccattaaatcttaattcccc

SEQ ID NO: 487 - DNA, 21 bp, Homo sapiens
accttacacctcaacccattt

SEQ ID NO: 488 - DNA, 27 bp, Homo sapiens
ctatatataaaacaaccccatttctca

SEQ ID NO: 489 - DNA, 24 bp, Homo sapiens
atactcacaacctctcacacaaaa

SEQ ID NO: 490 - DNA, 26 bp, Homo sapiens
ctttctcactttaacaccaactaccc

SEQ ID NO: 491 - DNA, 22 bp, Homo sapiens
aaacaaaacaatcccaccctac

SEQ ID NO: 492 - DNA, 23 bp, Homo sapiens
attaaacgcgaactacgataacc

SEQ ID NO: 493 - DNA, 23 bp, Homo sapiens
caaataaccaacaaaaacctcca

SEQ ID NO: 494 - DNA, 22 bp, Homo sapiens
cgcctaacacaacgcctaatac

SEQ ID NO: 495 - DNA, 26 bp, Homo sapiens
acaaaccaacccttctaatatactcc

SEQ ID NO: 496 - DNA, 23 bp, Homo sapiens
aacttcctaccccacccaataaa

SEQ ID NO: 497 - DNA, 21 bp, Homo sapiens
gactctcctcaactccttccc

SEQ ID NO: 498 - DNA, 21 bp, Homo sapiens
aaccccaatctccgcaataaa

SEQ ID NO: 499 - DNA, 22 bp, Homo sapiens
taccctatacccgcgatattcc

SEQ ID NO: 500 - DNA, 22 bp, Homo sapiens
aactaaaaaactccgaaacccc

SEQ ID NO: 501 - DNA, 24 bp, Homo sapiens
ctctacaatccatttatttacccg

SEQ ID NO: 502 - DNA, 21 bp, Homo sapiens
acccaaccacattaacgaacc

SEQ ID NO: 503 - DNA, 23 bp, Homo sapiens
tactaacgaactcctcgctccaa

SEQ ID NO: 504 - DNA, 22 bp, Homo sapiens
gaacataaaaccgaaaacaccc

SEQ ID NO: 505 - DNA, 23 bp, Homo sapiens
tccttactcgaatctttaccgaa

SEQ ID NO: 506 - DNA, 23 bp, Homo sapiens
ccaatctaaccttccaaacacat

SEQ ID NO: 507 - DNA, 21 bp, Homo sapiens
attaaaccttctctcgtcgcc

SEQ ID NO: 508 - DNA, 22 bp, Homo sapiens
ataaacacaacaccctacacgc

SEQ ID NO: 509 - DNA, 23 bp, Homo sapiens
gacacacacaatacaatcacgct

SEQ ID NO: 510 - DNA, 20 bp, Homo sapiens
acaactcccgaaacaaaacc

SEQ ID NO: 511 - DNA, 22 bp, Homo sapiens
ctcctcgccttccctttatatc

SEQ ID NO: 512 - DNA, 21 bp, Homo sapiens
taacaacgaccacgaacacaa

SEQ ID NO: 513 - DNA, 25 bp, Homo sapiens
aactcaatttcttccacctaaaaac

SEQ ID NO: 514 - DNA, 22 bp, Homo sapiens
ccaacttccgaccgaaaactac

SEQ ID NO: 515 - DNA, 23 bp, Homo sapiens
ataacgctcgaatccgactaaac

SEQ ID NO: 516 - DNA, 23 bp, Homo sapiens
gcgatcgacaactaacgacctaa

SEQ ID NO: 517 - DNA, 23 bp, Homo sapiens
accttataaaaaacacaaaccgc

SEQ ID NO: 518 - DNA, 22 bp, Homo sapiens
tatatccaacgaacatcgacca

SEQ ID NO: 519 - DNA, 22 bp, Homo sapiens
cctaaactacacttttccccct

SEQ ID NO: 520 - DNA, 24 bp, Homo sapiens
acaattacttcaaaacgtctccaa

SEQ ID NO: 521 - DNA, 21 bp, Homo sapiens
cctcccaaaccattcaaaaac

SEQ ID NO: 522 - DNA, 22 bp, Homo sapiens
gaccaaaacaatctcctccgac

SEQ ID NO: 523 - DNA, 22 bp, Homo sapiens
accccttaaaattccaaaaacc

SEQ ID NO: 524 - DNA, 26 bp, Homo sapiens
ccacctcctcaacttactctaacttc

SEQ ID NO: 525 - DNA, 18 bp, Homo sapiens
accgcaaacgctccataa

SEQ ID NO: 526 - DNA, 22 bp, Homo sapiens
ttcgcttaactaacgacgcaac

SEQ ID NO: 527 - DNA, 27 bp, Homo sapiens
atacaaaatatttaaaaaccctcacaa

SEQ ID NO: 528 - DNA, 25 bp, Homo sapiens
taactcctctcattccaatacctaa

SEQ ID NO: 529 - DNA, 23 bp, Homo sapiens
aatacccccacaactctaaacct

SEQ ID NO: 530 - DNA, 21 bp, Homo sapiens
ccaaaaaaccaatcaaaacca

SEQ ID NO: 531 - DNA, 21 bp, Homo sapiens
acgcaaaactccgaattcaat

SEQ ID NO: 532 - DNA, 22 bp, Homo sapiens
aaacgcatacgtaacgacaaca

SEQ ID NO: 533 - DNA, 23 bp, Homo sapiens
gatcccatttcgaaatttcctct

SEQ ID NO: 534 - DNA, 24 bp, Homo sapiens
attatcaaacgacgaacgtctacc

SEQ ID NO: 535 - DNA, 23 bp, Homo sapiens
ttcccctttacaaactaccaacc

SEQ ID NO: 536 - DNA, 23 bp, Homo sapiens
acaaccgaaaataaaaccaaacc

SEQ ID NO: 537 - DNA, 24 bp, Homo sapiens
aatcccaatcccttaattaaactt

SEQ ID NO: 538 - DNA, 22 bp, Homo sapiens
ccccatttctcactcccattaa

SEQ ID NO: 539 - DNA, 18 bp, Homo sapiens
aaacccccaacctacccc

SEQ ID NO: 540 - DNA, 22 bp, Homo sapiens
aatacaaatcacctctcccaaa

SEQ ID NO: 541 - DNA, 18 bp, Homo sapiens
accccacccaacttcaaa

SEQ ID NO: 542 - DNA, 23 bp, Homo sapiens
attcaaacgattctcctacctca

SEQ ID NO: 543 - DNA, 23 bp, Homo sapiens
aacttacccttccgattaaccct

SEQ ID NO: 544 - DNA, 23 bp, Homo sapiens
actcctaactcaaataatccgcc

SEQ ID NO: 545 - DNA, 23 bp, Homo sapiens
attaaatccaaacctccttcacc

SEQ ID NO: 546 - DNA, 22 bp, Homo sapiens
aatactaccccctccaaatccc

SEQ ID NO: 547 - DNA, 23 bp, Homo sapiens
ctacttaccgcacttccgaacta

SEQ ID NO: 548 - DNA, 22 bp, Homo sapiens
ccccatacccctcaaaacctat

SEQ ID NO: 549 - DNA, 19 bp, Homo sapiens
tttcttctccatcgcgaaa

SEQ ID NO: 550 - DNA, 26 bp, Homo sapiens
ttcctccaaataaactcataaatttc

SEQ ID NO: 551 - DNA, 21 bp, Homo sapiens
ccccgaaaactctatccaaaa

SEQ ID NO: 552 - DNA, 22 bp, Homo sapiens
accaaacttcactaaacgtccg

SEQ ID NO: 553 - DNA, 23 bp, Homo sapiens
acgtaacctcactttcctactcc

SEQ ID NO: 554 - DNA, 23 bp, Homo sapiens
ccctacccgaactcactacttac

SEQ ID NO: 555 - DNA, 24 bp, Homo sapiens
gatttcccctaatctcttcattca

SEQ ID NO: 556 - DNA, 24 bp, Homo sapiens
tacacttccaaaccttcaacaaac

SEQ ID NO: 557 - DNA, 24 bp, Homo sapiens
caaacacacacatacacacacaca

SEQ ID NO: 558 - DNA, 28 bp, Homo sapiens
aacattataattccaaccaaaaatactc

SEQ ID NO: 559 - DNA, 24 bp, Homo sapiens
taaccctactcttataacctcccg

SEQ ID NO: 560 - DNA, 25 bp, Homo sapiens
tcttccttcaactataacttacgcc

SEQ ID NO: 561 - DNA, 23 bp, Homo sapiens
actcaacctcgtaataacgccta

SEQ ID NO: 562 - DNA, 28 bp, Homo sapiens
caatttctcacctatacatttcctataa

SEQ ID NO: 563 - DNA, 20 bp, Homo sapiens
tcctcccctactacccctcg

SEQ ID NO: 564 - DNA, 18 bp, Homo sapiens
cccaaactaacgcatccg

SEQ ID NO: 565 - DNA, 24 bp, Homo sapiens
caaaacaatactaattccaacccc

SEQ ID NO: 566 - DNA, 22 bp, Homo sapiens
ccgttctaaacgaaaacccaac

SEQ ID NO: 567 - DNA, 23 bp, Homo sapiens
ctcaacgacctcaaaaactaccc

SEQ ID NO: 568 - DNA, 28 bp, Homo sapiens
aaaaactctataaaaataacaacgacct

SEQ ID NO: 569 - DNA, 19 bp, Homo sapiens
cccgcccaactataaaaaa

SEQ ID NO: 570 - DNA, 18 bp, Homo sapiens
ccgacgaacaacgctacg

SEQ ID NO: 571 - DNA, 21 bp, Homo sapiens
cactacgacccaaaacaatcc

SEQ ID NO: 572 - DNA, 23 bp, Homo sapiens
aactcacaaatccctttcctaac

SEQ ID NO: 573 - DNA, 22 bp, Homo sapiens
aaaaaacaaactccgaacgaaa

SEQ ID NO: 574 - DNA, 21 bp, Homo sapiens
tccccaacaatctctattccc

SEQ ID NO: 575 - DNA, 18 bp, Homo sapiens
cacgcccacttcctaccc

SEQ ID NO: 576 - DNA, 23 bp, Homo sapiens
cttcgcaaacctaaccaatcaat

SEQ ID NO: 577 - DNA, 23 bp, Homo sapiens
cgctaaaccaccccaaataaaac

SEQ ID NO: 578 - DNA, 21 bp, Homo sapiens
ctcccgaattcaaacgattct

SEQ ID NO: 579 - DNA, 24 bp, Homo sapiens
aatccgaaataaattccctaaact

SEQ ID NO: 580 - DNA, 23 bp, Homo sapiens
tctcacacttttcctcgaaaaat

SEQ ID NO: 581 - DNA, 23 bp, Homo sapiens
gcaaacctaaatacgtccctcct

SEQ ID NO: 582 - DNA, 22 bp, Homo sapiens
accttcattcgaacgcttaaac

SEQ ID NO: 583 - DNA, 23 bp, Homo sapiens
aactacttcctctccctccacaa

SEQ ID NO: 584 - DNA, 23 bp, Homo sapiens
ttcaaaaatcgaccaatcgtaat

SEQ ID NO: 585 - DNA, 21 bp, Homo sapiens
tcgcgaacaaattaacttccc

SEQ ID NO: 586 - DNA, 28 bp, Homo sapiens
ttaactacttactttcttctctccaccc

SEQ ID NO: 587 - DNA, 22 bp, Homo sapiens
aaaaccaatttactcgcgaacg

SEQ ID NO: 588 - DNA, 23 bp, Homo sapiens
caactaaacccaacgccaatcta

SEQ ID NO: 589 - DNA, 21 bp, Homo sapiens
aaacaatccaaacccgaaaac

SEQ ID NO: 590 - DNA, 19 bp, Homo sapiens
gaacgcatacgcgacaaac

SEQ ID NO: 591 - DNA, 21 bp, Homo sapiens
tcctaccctcaccctcctaaa

SEQ ID NO: 592 - DNA, 26 bp, Homo sapiens
attctcaaaataaaaattccctatca

SEQ ID NO: 593 - DNA, 22 bp, Homo sapiens
cattaccctttctatccccacc

SEQ ID NO: 594 - DNA, 27 bp, Homo sapiens
aaccaacctaaccaacataataaaacc

SEQ ID NO: 595 - DNA, 23 bp, Homo sapiens
taaactccaaacacccacctacc

SEQ ID NO: 596 - DNA, 21 bp, Homo sapiens
accctccgaacctccaactac

SEQ ID NO: 597 - DNA, 23 bp, Homo sapiens
ctacatctactcatttcctccca

SEQ ID NO: 598 - DNA, 27 bp, Homo sapiens
aatacaataacataacctctacctccc

SEQ ID NO: 599 - DNA, 24 bp, Homo sapiens
cctaaaaaacacaacaaaactcca

SEQ ID NO: 600 - DNA, 24 bp, Homo sapiens
ttaccacactctaaccaatcaaaa

SEQ ID NO: 601 - DNA, 23 bp, Homo sapiens
ctaccgaattcatttaaaaaccg

SEQ ID NO: 602 - DNA, 25 bp, Homo sapiens
ttccctttcttcacgatacttaaca

SEQ ID NO: 603 - DNA, 23 bp, Homo sapiens
ttcaactccgaaatttccaaact

SEQ ID NO: 604 - DNA, 21 bp, Homo sapiens
ttcgtcactcccacgattaac

SEQ ID NO: 605 - DNA, 22 bp, Homo sapiens
aaaacgaccaaaacaaaaccaa

SEQ ID NO: 606 - DNA, 21 bp, Homo sapiens
catcaaccaaacattccgaaa

SEQ ID NO: 607 - DNA, 19 bp, Homo sapiens
aaaacgcgtatcctcaccc

SEQ ID NO: 608 - DNA, 20 bp, Homo sapiens
accctcgctaactctcctcc

SEQ ID NO: 609 - DNA, 23 bp, Homo sapiens
caatccttaaaccaatcgacgta

SEQ ID NO: 610 - DNA, 23 bp, Homo sapiens
cgaaacattaaactcctcctcct

SEQ ID NO: 611 - DNA, 24 bp, Homo sapiens
taaacaacataacgtacgacaccg

SEQ ID NO: 612 - DNA, 22 bp, Homo sapiens
tcaactccgtttcgatttcact

SEQ ID NO: 613 - DNA, 21 bp, Homo sapiens
aacaatacgacctatcaccgc

SEQ ID NO: 614 - DNA, 23 bp, Homo sapiens
tactttaaaccgaaccaaaccaa

SEQ ID NO: 615 - DNA, 21 bp, Homo sapiens
aaaaattcaaactcccgaacg

SEQ ID NO: 616 - DNA, 23 bp, Homo sapiens
cttcttccgactcttcacctcaa

SEQ ID NO: 617 - DNA, 22 bp, Homo sapiens
gacgcgtccctaaaacttaacc

SEQ ID NO: 618 - DNA, 20 bp, Homo sapiens
ttcgccacaaaccttctttc

SEQ ID NO: 619 - DNA, 24 bp, Homo sapiens
acgaacaactaaactcgactaccg

SEQ ID NO: 620 - DNA, 23 bp, Homo sapiens
ctactaaaaaaccaatcaacgcg

SEQ ID NO: 621 - DNA, 26 bp, Homo sapiens
atcctaatcctcctacccttatattt

SEQ ID NO: 622 - DNA, 29 bp, Homo sapiens
aaactaactaaacaattaactctcctccc

SEQ ID NO: 623 - DNA, 23 bp, Homo sapiens
aattctaaacgtttcccgactac

SEQ ID NO: 624 - DNA, 20 bp, Homo sapiens
actacaaccgacgaaccacg

SEQ ID NO: 625 - DNA, 19 bp, Homo sapiens
gacccgaaactaccgcaaa

SEQ ID NO: 626 - DNA, 23 bp, Homo sapiens
aatcctaaacctaaacctcccga

SEQ ID NO: 627 - DNA, 20 bp, Homo sapiens
accgaatcttcccttatccc

SEQ ID NO: 628 - DNA, 23 bp, Homo sapiens
tccttacaacctctaacctcctt

SEQ ID NO: 629 - DNA, 24 bp, Homo sapiens
cctcttaaaacctacctacccctc

SEQ ID NO: 630 - DNA, 26 bp, Homo sapiens
aaattacaccaattataaaccgaacg

SEQ ID NO: 631 - DNA, 21 bp, Homo sapiens
gaaaccctctacccgcctatt

SEQ ID NO: 632 - DNA, 20 bp, Homo sapiens
aaaatcgaaaccaaaacccc

SEQ ID NO: 633 - DNA, 24 bp, Homo sapiens
ccgtaacaactacacgactcctaa

SEQ ID NO: 634 - DNA, 22 bp, Homo sapiens
cctcccgaactaaaacgattct

SEQ ID NO: 635 - DNA, 22 bp, Homo sapiens
caaaacgaaaccctatctccaa

SEQ ID NO: 636 - DNA, 22 bp, Homo sapiens
accatcccaactccctatcttt

SEQ ID NO: 637 - DNA, 20 bp, Homo sapiens
acgaaactctacgacgacgc

SEQ ID NO: 638 - DNA, 22 bp, Homo sapiens
aaaacgaaaccgcgactataaa

SEQ ID NO: 639 - DNA, 23 bp, Homo sapiens
taaaatcaccacccgaaaaacat

SEQ ID NO: 640 - DNA, 26 bp, Homo sapiens
tttaataaaattttccctcccttacc

SEQ ID NO: 641 - DNA, 22 bp, Homo sapiens
cttctactcaaacaccaacgcc

SEQ ID NO: 642 - DNA, 24 bp, Homo sapiens
aaaatcaaatccaaataacatccc

SEQ ID NO: 643 - DNA, 23 bp, Homo sapiens
ctcccgccactttactaaaaact

SEQ ID NO: 644 - DNA, 21 bp, Homo sapiens
taccacttcccaactcttccc

SEQ ID NO: 645 - DNA, 22 bp, Homo sapiens
gccctacatcccaacactaaaa

SEQ ID NO: 646 - DNA, 25 bp, Homo sapiens
aaatacctacgaactcctaaaaccc

SEQ ID NO: 647 - DNA, 18 bp, Homo sapiens
gacgacgcccatatcgac

SEQ ID NO: 648 - DNA, 22 bp, Homo sapiens
aacctctcccaacctcaatttt

SEQ ID NO: 649 - DNA, 22 bp, Homo sapiens
actaataaccgctctcgcttct

SEQ ID NO: 650 - DNA, 27 bp, Homo sapiens
aaacaaactcgctaaaatctaaataac

SEQ ID NO: 651 - DNA, 27 bp, Homo sapiens
tttttaaaaaacctataaaaccaatcc

SEQ ID NO: 652 - DNA, 17 bp, Homo sapiens
aaccatcctccgcctca

SEQ ID NO: 653 - DNA, 23 bp, Homo sapiens
cctacaaccctactacccctacc

SEQ ID NO: 654 - DNA, 23 bp, Homo sapiens
ctaacacaaaaaccacactccaa

SEQ ID NO: 655 - DNA, 24 bp, Homo sapiens
cccaaaaactaaacttcctctaaa

SEQ ID NO: 656 - DNA, 20 bp, Homo sapiens
acactaccaccaccaccatc

SEQ ID NO: 657 - DNA, 23 bp, Homo sapiens
aaacatttcgcaattcctatctc

SEQ ID NO: 658 - DNA, 23 bp, Homo sapiens
atattctctctcattctacgccg

SEQ ID NO: 659 - DNA, 25 bp, Homo sapiens
aatcctatactcgataccgtttctc

SEQ ID NO: 660 - DNA, 24 bp, Homo sapiens
acaaccacaccaaaccctatatct

SEQ ID NO: 661 - DNA, 23 bp, Homo sapiens
acttccaacccttaaaacaaaca

SEQ ID NO: 662 - DNA, 22 bp, Homo sapiens
tcccaaaaaacaacttccctaa

SEQ ID NO: 663 - DNA, 23 bp, Homo sapiens
ttattttaaacccaatcttccct

SEQ ID NO: 664 - DNA, 25 bp, Homo sapiens
ttacaatataatcctccccaaaatc

SEQ ID NO: 665 - DNA, 24 bp, Homo sapiens
caaatttcccaatttaattcctca

SEQ ID NO: 666 - DNA, 23 bp, Homo sapiens
gattaaacgcgacgctcaattac

SEQ ID NO: 667 - DNA, 21 bp, Homo sapiens
cgacacacaaaaaccgactac

SEQ ID NO: 668 - DNA, 25 bp, Homo sapiens
ataccattacaaaaacaacccaaca

SEQ ID NO: 669 - DNA, 24 bp, Homo sapiens
acacatatcccctctctaatcctc

SEQ ID NO: 670 - DNA, 23 bp, Homo sapiens
aaaccccatacccttaaacccta

SEQ ID NO: 671 - DNA, 23 bp, Homo sapiens
acctcaatcttctcgtcaacaaa

SEQ ID NO: 672 - DNA, 21 bp, Homo sapiens
cccaaccaataaacgacgaaa

SEQ ID NO: 673 - DNA, 21 bp, Homo sapiens
cctcacgaactaccctcaaac

SEQ ID NO: 674 - DNA, 23 bp, Homo sapiens
taaacaccaacttctctcccttt

SEQ ID NO: 675 - DNA, 23 bp, Homo sapiens
ctcctcccctactcattttaaaa

SEQ ID NO: 676 - DNA, 19 bp, Homo sapiens
tttccaaactaaaccgccg

SEQ ID NO: 677 - DNA, 27 bp, Homo sapiens
taacacctaattttatataccccaacc

SEQ ID NO: 678 - DNA, 23 bp, Homo sapiens
actccgtctaacttctttctccc

SEQ ID NO: 679 - DNA, 20 bp, Homo sapiens
aactttcaatacatccgccg

SEQ ID NO: 680 - DNA, 23 bp, Homo sapiens
caaaacccataaaaattcaaaaa

SEQ ID NO: 681 - DNA, 17 bp, Homo sapiens
cccctactcctccccac

SEQ ID NO: 682 - DNA, 18 bp, Homo sapiens
ttcctcctccaacccctc

SEQ ID NO: 683 - DNA, 21 bp, Homo sapiens
ccctaacacgactctacccaa

SEQ ID NO: 684 - DNA, 21 bp, Homo sapiens
cctccccataccaacttaaaa

SEQ ID NO: 685 - DNA, 26 bp, Homo sapiens
aatcatctaaaataaaaacacccaac

SEQ ID NO: 686 - DNA, 23 bp, Homo sapiens
accaaatctataccaaaatcccc

SEQ ID NO: 687 - DNA, 23 bp, Homo sapiens
aaattaactcctcaaaacacccg

SEQ ID NO: 688 - DNA, 23 bp, Homo sapiens
aaacccaaaactacctccaaatc

SEQ ID NO: 689 - DNA, 22 bp, Homo sapiens
aaccaaaccgaactcccaataa

SEQ ID NO: 690 - DNA, 20 bp, Homo sapiens
ttcgacctctctccgctaaa

SEQ ID NO: 691 - DNA, 23 bp, Homo sapiens
acccatctcccgttacataaaac

SEQ ID NO: 692 - DNA, 22 bp, Homo sapiens
ctaaacacccgaaccaacgact

SEQ ID NO: 693 - DNA, 20 bp, Homo sapiens
accacatacgccaaataccc

SEQ ID NO: 694 - DNA, 23 bp, Homo sapiens
cctcaacaaataaaccccaaaat

SEQ ID NO: 695 - DNA, 21 bp, Homo sapiens
ccgcccctcaactatttattc

SEQ ID NO: 696 - DNA, 22 bp, Homo sapiens
ttccaatcgcattacgtaaaca

SEQ ID NO: 697 - DNA, 28 bp, Homo sapiens
tctaaaaacctaccactaatattcacaa

SEQ ID NO: 698 - DNA, 25 bp, Homo sapiens
taaaccctaaactaccataaaaccg

SEQ ID NO: 699 - DNA, 28 bp, Homo sapiens
aaaattaactactctcacctaacaaacc

SEQ ID NO: 700 - DNA, 22 bp, Homo sapiens
tttcataacaacccaaaatcca

SEQ ID NO: 701 - DNA, 24 bp, Homo sapiens
aaacactaaccgtaaaccaaacac

SEQ ID NO: 702 - DNA, 23 bp, Homo sapiens
caacgctatacctaaacgacctt

SEQ ID NO: 703 - DNA, 22 bp, Homo sapiens
aaacttcgactaacaaaccccg

SEQ ID NO: 704 - DNA, 23 bp, Homo sapiens
cttatcccaaaaaacaaaaccaa

SEQ ID NO: 705 - DNA, 24 bp, Homo sapiens
ttccaaccctcacctaactttatc

SEQ ID NO: 706 - DNA, 25 bp, Homo sapiens
cctattcaaatcaactccttctaaa

SEQ ID NO: 707 - DNA, 20 bp, Homo sapiens
cacaaacaaacgctccaaaa

SEQ ID NO: 708 - DNA, 23 bp, Homo sapiens
cccaaaatctccgtaaactaaaa

SEQ ID NO: 709 - DNA, 23 bp, Homo sapiens
aaaatccctctcacccattaaca

SEQ ID NO: 710 - DNA, 20 bp, Homo sapiens
aaaaaccgcgaccctaaaaa

SEQ ID NO: 711 - DNA, 23 bp, Homo sapiens
gacctcttcgaacaacccaaata

SEQ ID NO: 712 - DNA, 23 bp, Homo sapiens
ccctatcctaatcgcccaactaa

SEQ ID NO: 713 - DNA, 23 bp, Homo sapiens
ccaccaaacatacaattccaact

SEQ ID NO: 714 - DNA, 23 bp, Homo sapiens
attctatccctatcctaatcgcc

SEQ ID NO: 715 - DNA, 20 bp, Homo sapiens
actctctaaaaccgccgtcg

SEQ ID NO: 716 - DNA, 23 bp, Homo sapiens
gtaaacctccccctcaactaaaa

SEQ ID NO: 717 - DNA, 22 bp, Homo sapiens
cgctacctaacgctctaactcc

SEQ ID NO: 718 - DNA, 26 bp, Homo sapiens
tccaattccctaatatacctatttca

SEQ ID NO: 719 - DNA, 21 bp, Homo sapiens
accaacacaatctctacgccc

SEQ ID NO: 720 - DNA, 22 bp, Homo sapiens
aacctcgaaccttaacaattcg

SEQ ID NO: 721 - DNA, 22 bp, Homo sapiens
aattccctacactcatcccctt

SEQ ID NO: 722 - DNA, 20 bp, Homo sapiens
ccccactaaacccttaaccc

SEQ ID NO: 723 - DNA, 19 bp, Homo sapiens
aaaactctcccctctcccc

SEQ ID NO: 724 - DNA, 25 bp, Homo sapiens
aaaatccaaactccttacctctaat

SEQ ID NO: 725 - DNA, 27 bp, Homo sapiens
taaacattctatacctacttcccaaaa

SEQ ID NO: 726 - DNA, 28 bp, Homo sapiens
taaacaataccaactaattccacttttc

SEQ ID NO: 727 - DNA, 21 bp, Homo sapiens
attccttcctaacgctccctc

SEQ ID NO: 728 - DNA, 21 bp, Homo sapiens
aactcgctcctcgcgaaatac

SEQ ID NO: 729 - DNA, 27 bp, Homo sapiens
aattctaaaaatccgaaattctaaacc

SEQ ID NO: 730 - DNA, 22 bp, Homo sapiens
atttctaaaaccccgaaacctc

SEQ ID NO: 731 - DNA, 23 bp, Homo sapiens
tctatacccgctaatacaaaccc

SEQ ID NO: 732 - DNA, 25 bp, Homo sapiens
aacacacacacaaaaaacctataca

SEQ ID NO: 733 - DNA, 21 bp, Homo sapiens
acatacacgcacactcaaaca

SEQ ID NO: 734 - DNA, 21 bp, Homo sapiens
cccccttaaaccctaaactca

SEQ ID NO: 735 - DNA, 18 bp, Homo sapiens
cccacgcaaaaccgaaaa

SEQ ID NO: 736 - DNA, 21 bp, Homo sapiens
tcacgtcaacctccccaaata

SEQ ID NO: 737 - DNA, 20 bp, Homo sapiens
aataccaccttccgcaaacc

SEQ ID NO: 738 - DNA, 20 bp, Homo sapiens
aacccaatctcacaacaccc

SEQ ID NO: 739 - DNA, 23 bp, Homo sapiens
aaaacctactcgcgaccttaact

SEQ ID NO: 740 - DNA, 22 bp, Homo sapiens
gacccaaccaataaaccaaaac

SEQ ID NO: 741 - DNA, 24 bp, Homo sapiens
ctaactccctaccattatccacca

SEQ ID NO: 742 - DNA, 23 bp, Homo sapiens
actccttacccactccgaactaa

SEQ ID NO: 743 - DNA, 23 bp, Homo sapiens
aaccaacccaaattcaaaactaa

SEQ ID NO: 744 - DNA, 25 bp, Homo sapiens
aattcctaactccctaatccctacc

SEQ ID NO: 745 - DNA, 22 bp, Homo sapiens
cctaactcaaccataaacccca

SEQ ID NO: 746 - DNA, 25 bp, Homo sapiens
aaattctatactcccctcaaaaatc

SEQ ID NO: 747 - DNA, 25 bp, Homo sapiens
aaccttccaaatactaccctaactc

SEQ ID NO: 748 - DNA, 22 bp, Homo sapiens
caaaacgaaaccctatctctcg

SEQ ID NO: 749 - DNA, 25 bp, Homo sapiens
gcctaaccaatcttctcttcttaca

SEQ ID NO: 750 - DNA, 20 bp, Homo sapiens
tcctccaacctaaacctccc

SEQ ID NO: 751 - DNA, 23 bp, Homo sapiens
aacaccctcacacttctacacca

SEQ ID NO: 752 - DNA, 25 bp, Homo sapiens
cttccaactttatcacacacctaca

SEQ ID NO: 753 - DNA, 25 bp, Homo sapiens
cccaacttccaaacaataataaaca

SEQ ID NO: 754 - DNA, 26 bp, Homo sapiens
tccctaataaatcaaaacatctacca

SEQ ID NO: 755 - DNA, 22 bp, Homo sapiens
aactttcaactacccctcacca

SEQ ID NO: 756 - DNA, 23 bp, Homo sapiens
acatccaacccctaactctctca

SEQ ID NO: 757 - DNA, 24 bp, Homo sapiens
taacacccaaatctctcctaacca

SEQ ID NO: 758 - DNA, 24 bp, Homo sapiens
ccaactctatacccaacacctaca

SEQ ID NO: 759 - DNA, 24 bp, Homo sapiens
aaccctaacaccaaacctacttca

SEQ ID NO: 760 - DNA, 24 bp, Homo sapiens
ccaccaaatatcttttcttcttca

SEQ ID NO: 761 - DNA, 25 bp, Homo sapiens
aatctatcccaacataacttcctca

SEQ ID NO: 762 - DNA, 24 bp, Homo sapiens
acaccaacctaacaaaacaactca

SEQ ID NO: 763 - DNA, 24 bp, Homo sapiens
aaaccttcttaccttcaaacctca

SEQ ID NO: 764 - DNA, 23 bp, Homo sapiens
aaaaatactcccattctccacca

SEQ ID NO: 765 - DNA, 22 bp, Homo sapiens
cactaccactccccaaaaaaca

SEQ ID NO: 766 - DNA, 23 bp, Homo sapiens
aaaccaacaaaaataccccaaca

SEQ ID NO: 767 - DNA, 24 bp, Homo sapiens
actccacactcaaccaattaaaca

SEQ ID NO: 768 - DNA, 23 bp, Homo sapiens
ccaattcccaacataacaacaca

SEQ ID NO: 769 - DNA, 22 bp, Homo sapiens
aactcacccctcctttctacca

SEQ ID NO: 770 - DNA, 23 bp, Homo sapiens
acccacatccaaattcctaaaca

SEQ ID NO: 771 - DNA, 23 bp, Homo sapiens
tccaacccacaaaatttaaaaca

SEQ ID NO: 772 - DNA, 25 bp, Homo sapiens
aacaatacaaccaacattcctaaca

SEQ ID NO: 773 - DNA, 23 bp, Homo sapiens
caactcctaatccccaataaaca

SEQ ID NO: 774 - DNA, 24 bp, Homo sapiens
aacatctttaaacaaacttcccca

SEQ ID NO: 775 - DNA, 22 bp, Homo sapiens
aaaacaaacaacctccctctca

SEQ ID NO: 776 - DNA, 23 bp, Homo sapiens
acctttaatttctcccaaaccca

SEQ ID NO: 777 - DNA, 24 bp, Homo sapiens
caaaaatctctactaactccccca

SEQ ID NO: 778 - DNA, 23 bp, Homo sapiens
caacaacacctctcacacataca

SEQ ID NO: 779 - DNA, 24 bp, Homo sapiens
aacaaaacaacaaaacaataccca

SEQ ID NO: 780 - DNA, 24 bp, Homo sapiens
caccaaaaacctaaccactactca

SEQ ID NO: 781 - DNA, 23 bp, Homo sapiens
cttccccttccctcaactaaaca

SEQ ID NO: 782 - DNA, 23 bp, Homo sapiens
ctcctactccttctcctaatcca

SEQ ID NO: 783 - DNA, 25 bp, Homo sapiens
aactaaaacaacttctcccaaaaca

SEQ ID NO: 784 - DNA, 27 bp, Homo sapiens
attaatatctacaacaccaaatcaaca

SEQ ID NO: 785 - DNA, 23 bp, Homo sapiens
ctccatttcctttacttcctcca

SEQ ID NO: 786 - DNA, 21 bp, Homo sapiens
ccccaccacctacaaaaaaca

SEQ ID NO: 787 - DNA, 23 bp, Homo sapiens
ccacaaaaaccaaaaaactacca

SEQ ID NO: 788 - DNA, 23 bp, Homo sapiens
taaataaccaatccaaaacccca

SEQ ID NO: 789 - DNA, 25 bp, Homo sapiens
ctcaaataaaattcttcctcttcca

SEQ ID NO: 790 - DNA, 28 bp, Homo sapiens
aaactcaaatcaataacactactcaaca

SEQ ID NO: 791 - DNA, 24 bp, Homo sapiens
aaaatattccccttacaaaaacca

SEQ ID NO: 792 - DNA, 28 bp, Homo sapiens
aaaacaacctactattatatcaccaaca

SEQ ID NO: 793 - DNA, 26 bp, Homo sapiens
caactctacataaatacacaaaccca

SEQ ID NO: 794 - DNA, 23 bp, Homo sapiens
atcaaccaaacctacatcccaca

SEQ ID NO: 795 - DNA, 25 bp, Homo sapiens
ccacaaaaaacctaaaaacaaaaca

SEQ ID NO: 796 - DNA, 25 bp, Homo sapiens
cccatacacttcctaaataaaacca

SEQ ID NO: 797 - DNA, 24 bp, Homo sapiens
ctctttctctaataacccacacca

SEQ ID NO: 798 - DNA, 21 bp, Homo sapiens
cccaacctaactctccttcca

SEQ ID NO: 799 - DNA, 24 bp, Homo sapiens
tctcttaaatcttaacaatcccca

SEQ ID NO: 800 - DNA, 25 bp, Homo sapiens
tccctaaatacctcctacacaaaca

SEQ ID NO: 801 - DNA, 27 bp, Homo sapiens
caataatcaatcacataaaacattcca

SEQ ID NO: 802 - DNA, 24 bp, Homo sapiens
acacaccaacctctaacaatacca

SEQ ID NO: 803 - DNA, 25 bp, Homo sapiens
aactcaaaaccataattttcaaaca

SEQ ID NO: 804 - DNA, 24 bp, Homo sapiens
taaaactaacctctcctaccctca

SEQ ID NO: 805 - DNA, 27 bp, Homo sapiens
tcctaaaacaaataacacaataccaca

SEQ ID NO: 806 - DNA, 24 bp, Homo sapiens
ccaataaaaacaaaacccaactca

SEQ ID NO: 807 - DNA, 24 bp, Homo sapiens
cacaaatcaataacatcacatcca

SEQ ID NO: 808 - DNA, 21 bp, Homo sapiens
ctcaaccctaatacccatcca

SEQ ID NO: 809 - DNA, 21 bp, Homo sapiens
ccactccaatactcactccca

SEQ ID NO: 810 - DNA, 24 bp, Homo sapiens
aacaaacaaaacaaacaaaatcca

SEQ ID NO: 811 - DNA, 26 bp, Homo sapiens
aaaaccttaactaacacaaaaccaca

SEQ ID NO: 812 - DNA, 25 bp, Homo sapiens
ccaaatctcactcctaaattcaaca

SEQ ID NO: 813 - DNA, 24 bp, Homo sapiens
tccacaaacctacaaaacctaaca

SEQ ID NO: 814 - DNA, 28 bp, Homo sapiens
caaaaactcaataataataaacacacca

SEQ ID NO: 815 - DNA, 23 bp, Homo sapiens
cccaataaaaccaacttaaccca

SEQ ID NO: 816 - DNA, 23 bp, Homo sapiens
ccttccttaaactctctccacca

SEQ ID NO: 817 - DNA, 23 bp, Homo sapiens
aataacaaccatcctacctccca

SEQ ID NO: 818 - DNA, 24 bp, Homo sapiens
ccaaccaaccataaaaataaaaca

SEQ ID NO: 819 - DNA, 23 bp, Homo sapiens
cctctcccctaccctataaaaca

SEQ ID NO: 820 - DNA, 23 bp, Homo sapiens
caaccaactacacaacaactcca

SEQ ID NO: 821 - DNA, 24 bp, Homo sapiens
caaaaaacaccttccataaaaaca

SEQ ID NO: 822 - DNA, 24 bp, Homo sapiens
accacaataccataaacatcacca

SEQ ID NO: 823 - DNA, 27 bp, Homo sapiens
aaactacaaattacacaatccaattca

SEQ ID NO: 824 - DNA, 27 bp, Homo sapiens
aataattaataaccaaccaaaaactca

SEQ ID NO: 825 - DNA, 21 bp, Homo sapiens
tctctttccctttctcttcca

SEQ ID NO: 826 - DNA, 26 bp, Homo sapiens
cataataaatttcacctctcaatcca

SEQ ID NO: 827 - DNA, 25 bp, Homo sapiens
aaaaatcctttacccctaaattaca

SEQ ID NO: 828 - DNA, 22 bp, Homo sapiens
ccacaccaacctaaactcaaca

SEQ ID NO: 829 - DNA, 23 bp, Homo sapiens
acccaactaaaactaaaacccca

SEQ ID NO: 830 - DNA, 23 bp, Homo sapiens
accacctcctaataaaaacccca

SEQ ID NO: 831 - DNA, 24 bp, Homo sapiens
ttacctctctccaaatctctcaca

SEQ ID NO: 832 - DNA, 28 bp, Homo sapiens
ctttctacatatctctcaacctctttca

SEQ ID NO: 833 - DNA, 25 bp, Homo sapiens
aaaatatcaaaacctctacaaccca

SEQ ID NO: 834 - DNA, 23 bp, Homo sapiens
caaacccaataaaatcaaaacca

SEQ ID NO: 835 - DNA, 23 bp, Homo sapiens
accctcaaccaatcaacaataca

SEQ ID NO: 836 - DNA, 25 bp, Homo sapiens
cctttaaaaaccatctaatcaacca

SEQ ID NO: 837 - DNA, 23 bp, Homo sapiens
aactccattctataccacaccca

SEQ ID NO: 838 - DNA, 25 bp, Homo sapiens
cttacttcattctctacacaaccca

SEQ ID NO: 839 - DNA, 23 bp, Homo sapiens
cctcctcctaatcctacacaaca

SEQ ID NO: 840 - DNA, 25 bp, Homo sapiens
ccatcaacactcaaaacaaactaca

SEQ ID NO: 841 - DNA, 23 bp, Homo sapiens
acccaactccaaactcataaaca

SEQ ID NO: 842 - DNA, 23 bp, Homo sapiens
aaactcctctacacctaccacca

SEQ ID NO: 843 - DNA, 23 bp, Homo sapiens
aaaccaatacaatccatatccca

SEQ ID NO: 844 - DNA, 24 bp, Homo sapiens
aaaaccaaaacttacaaaccaaca

SEQ ID NO: 845 - DNA, 25 bp, Homo sapiens
aatttatccccaattaataattcca

SEQ ID NO: 846 - DNA, 24 bp, Homo sapiens
cccttttataactccatctccaca

SEQ ID NO: 847 - DNA, 24 bp, Homo sapiens
ccactaccctatacacctaaccca

SEQ ID NO: 848 - DNA, 23 bp, Homo sapiens
aaccaacataccaacatccaaca

SEQ ID NO: 849 - DNA, 25 bp, Homo sapiens
aaccactcaaacacataacaaatca

SEQ ID NO: 850 - DNA, 24 bp, Homo sapiens
ccctttcctaattaacaccaaaca

SEQ ID NO: 851 - DNA, 24 bp, Homo sapiens
aaaaccaaaccaaaactaaatcca

SEQ ID NO: 852 - DNA, 24 bp, Homo sapiens
caataacccccaaaactaaactca

SEQ ID NO: 853 - DNA, 25 bp, Homo sapiens
aacaaaaacaaatccaaatatacca

SEQ ID NO: 854 - DNA, 23 bp, Homo sapiens
caaccctaaacatctaaacacca

SEQ ID NO: 855 - DNA, 18 bp, Homo sapiens
tcattccactccaaccca

SEQ ID NO: 856 - DNA, 25 bp, Homo sapiens
tctaaaaaatctccctcttatctca

SEQ ID NO: 857 - DNA, 19 bp, Homo sapiens
caacccttccaccctacca

SEQ ID NO: 858 - DNA, 24 bp, Homo sapiens
cacaaatcctaaaataccaaaaca

SEQ ID NO: 859 - DNA, 26 bp, Homo sapiens
caaaactacaataaaccaaactcaca

SEQ ID NO: 860 - DNA, 24 bp, Homo sapiens
cccatataaaatacaccaccctca

SEQ ID NO: 861 - DNA, 21 bp, Homo sapiens
tcccatcccctaataatcaca

SEQ ID NO: 862 - DNA, 29 bp, Homo sapiens
cataacaataacaaacatctcttatacca

SEQ ID NO: 863 - DNA, 24 bp, Homo sapiens
aaactataacccaacccaactaca

SEQ ID NO: 864 - DNA, 26 bp, Homo sapiens
aaacactaaaccataaaatctcaaca

SEQ ID NO: 865 - DNA, 25 bp, Homo sapiens
aaccctacaatctacacaaatacca

SEQ ID NO: 866 - DNA, 27 bp, Homo sapiens
ccctatattcacacacaatactattca

SEQ ID NO: 867 - DNA, 25 bp, Homo sapiens
ctaaccaacctaacaactctcaaca

SEQ ID NO: 868 - DNA, 21 bp, Homo sapiens
cacaccacacacacacacaca

SEQ ID NO: 869 - DNA, 26 bp, Homo sapiens
caaaaccaattatctttataaccaca

SEQ ID NO: 870 - DNA, 25 bp, Homo sapiens
cacatacacaaaaaacaaaaacaca

SEQ ID NO: 871 - DNA, 23 bp, Homo sapiens
tcctcaccccaaataaaaactca

SEQ ID NO: 872 - DNA, 23 bp, Homo sapiens
cctacacccaaatttccattaca

SEQ ID NO: 873 - DNA, 23 bp, Homo sapiens
aaatcacaatccaacctctacca

SEQ ID NO: 874 - DNA, 24 bp, Homo sapiens
aatacccaaaaacaataaccaaca

SEQ ID NO: 875 - DNA, 25 bp, Homo sapiens
tcaaacaacaaaacaataacctaca

SEQ ID NO: 876 - DNA, 28 bp, Homo sapiens
tcttcatcaaaacatctataaatcatca

SEQ ID NO: 877 - DNA, 24 bp, Homo sapiens
actccaaaactacccaaacctaca

SEQ ID NO: 878 - DNA, 23 bp, Homo sapiens
actccacaatccctaaacttcca

SEQ ID NO: 879 - DNA, 25 bp, Homo sapiens
aacacaacttatccctacttaccca

SEQ ID NO: 880 - DNA, 24 bp, Homo sapiens
attaacccaaacaataaccctaca

SEQ ID NO: 881 - DNA, 23 bp, Homo sapiens
tttctctcaaaccacttatccca

SEQ ID NO: 882 - DNA, 23 bp, Homo sapiens
cacaaaaccaaaaactcctacca

SEQ ID NO: 883 - DNA, 26 bp, Homo sapiens
acacaatccctttaaatactcataca

SEQ ID NO: 884 - DNA, 23 bp, Homo sapiens
ccccaaaaacactcaaatactca

SEQ ID NO: 885 - DNA, 23 bp, Homo sapiens
acaaacacaaacaaacccacaca

SEQ ID NO: 886 - DNA, 23 bp, Homo sapiens
aaacaaacccaacctcttactca

SEQ ID NO: 887 - DNA, 27 bp, Homo sapiens
caaacaaaccaaaatctttataacaca

SEQ ID NO: 888 - DNA, 24 bp, Homo sapiens
aacaaatcacctttacctcttcca

SEQ ID NO: 889 - DNA, 24 bp, Homo sapiens
acccactaaaaacaaaactcatca

SEQ ID NO: 890 - DNA, 24 bp, Homo sapiens
aaacaataaaccctacctccaaca

SEQ ID NO: 891 - DNA, 23 bp, Homo sapiens
atcctctacatcctcaccaaaca

SEQ ID NO: 892 - DNA, 26 bp, Homo sapiens
aaccaatactcactaaacacaacaca

SEQ ID NO: 893 - DNA, 29 bp, Homo sapiens
cacaacctataccctttataaaatacaca

SEQ ID NO: 894 - DNA, 29 bp, Homo sapiens
tctacttcttaacactaacctcaataaca

SEQ ID NO: 895 - DNA, 24 bp, Homo sapiens
ctacctaaccatcctcaaatttca

SEQ ID NO: 896 - DNA, 24 bp, Homo sapiens
ccttcctcaaaatctaacaattca

SEQ ID NO: 897 - DNA, 24 bp, Homo sapiens
ccactccaaataaaactaccacca

SEQ ID NO: 898 - DNA, 23 bp, Homo sapiens
cccacccaataatcaaataaaca

SEQ ID NO: 899 - DNA, 24 bp, Homo sapiens
cacccataattaccataatttcca

SEQ ID NO: 900 - DNA, 23 bp, Homo sapiens
accacacccaacctaataccaca

SEQ ID NO: 901 - DNA, 26 bp, Homo sapiens
cacaaacaataaacacaaactctaca

SEQ ID NO: 902 - DNA, 20 bp, Homo sapiens
aaaacccaacataacccaca

SEQ ID NO: 903 - DNA, 22 bp, Homo sapiens
aaaataaccctatccttcccca

SEQ ID NO: 904 - DNA, 22 bp, Homo sapiens
cccataaacacccaaatatcca

SEQ ID NO: 905 - DNA, 29 bp, Homo sapiens
actcaacatattaaaacatattcctaaca

SEQ ID NO: 906 - DNA, 25 bp, Homo sapiens
cccacattacttaaaaactcaaaca

SEQ ID NO: 907 - DNA, 24 bp, Homo sapiens
acaatcaaataaccacacaacaca

SEQ ID NO: 908 - DNA, 24 bp, Homo sapiens
caacctaaccctttaaatcttcca

SEQ ID NO: 909 - DNA, 23 bp, Homo sapiens
tctttttcttaaccctaccccca

SEQ ID NO: 910 - DNA, 25 bp, Homo sapiens
cctacccatacaatactaaaatcca

SEQ ID NO: 911 - DNA, 28 bp, Homo sapiens
ttatattattaatcaccaacaaacaaca

SEQ ID NO: 912 - DNA, 24 bp, Homo sapiens
catctcccttcattattcattcca

SEQ ID NO: 913 - DNA, 27 bp, Homo sapiens
tccttcttcctaaatactcataacaca

SEQ ID NO: 914 - DNA, 22 bp, Homo sapiens
caacctaacccaacaaacaaca

SEQ ID NO: 915 - DNA, 24 bp, Homo sapiens
aactctacaaatcccaaaatctca

SEQ ID NO: 916 - DNA, 26 bp, Homo sapiens
acacaaaacccaactatataaaaaca

SEQ ID NO: 917 - DNA, 24 bp, Homo sapiens
actcactacaacctctacctccca

SEQ ID NO: 918 - DNA, 23 bp, Homo sapiens
ccacataatcacacaacatctca

SEQ ID NO: 919 - DNA, 27 bp, Homo sapiens
ttcaccatattaaccaaactaatctca

SEQ ID NO: 920 - DNA, 24 bp, Homo sapiens
cttttaaccctaacctttaaccca

SEQ ID NO: 921 - DNA, 23 bp, Homo sapiens
ccctaacttttaacctttcccca

SEQ ID NO: 922 - DNA, 25 bp, Homo sapiens
ccaaaacaaaacttacttcacaaca

SEQ ID NO: 923 - DNA, 23 bp, Homo sapiens
aaaaccctaaaaactaaacccca

SEQ ID NO: 924 - DNA, 27 bp, Homo sapiens
ataataaaccctacaccttctatctca

SEQ ID NO: 925 - DNA, 24 bp, Homo sapiens
caaaaaatacaaaacccactacca

SEQ ID NO: 926 - DNA, 24 bp, Homo sapiens
cacaacctaaaaattacccatcca

SEQ ID NO: 927 - DNA, 24 bp, Homo sapiens
tccccttcattacaacaaactaca

SEQ ID NO: 928 - DNA, 23 bp, Homo sapiens
aaacaactctcaaaccctatcca

SEQ ID NO: 929 - DNA, 23 bp, Homo sapiens
catctctactcccaccacattca

SEQ ID NO: 930 - DNA, 23 bp, Homo sapiens
tcaatcatatttccaaaatccca

SEQ ID NO: 931 - DNA, 26 bp, Homo sapiens
ttcctaatcaccctctatcttctaca

SEQ ID NO: 932 - DNA, 24 bp, Homo sapiens
ataaaaaatcaccctaaccaaaca

SEQ ID NO: 933 - DNA, 27 bp, Homo sapiens
aataaataacctaatcacttcaaacca

SEQ ID NO: 934 - DNA, 24 bp, Homo sapiens
tctccccttacaacctttctaaca

SEQ ID NO: 935 - DNA, 24 bp, Homo sapiens
tcacctcaatacctcatactcaca

SEQ ID NO: 936 - DNA, 26 bp, Homo sapiens
ccaccacataccactcataatattca

SEQ ID NO: 937 - DNA, 23 bp, Homo sapiens
ttaacactcaatcccccttaaca

SEQ ID NO: 938 - DNA, 23 bp, Homo sapiens
aaaacaaattcaaacccaacaca

SEQ ID NO: 939 - DNA, 24 bp, Homo sapiens
caatcaacccaataaaacacaaca

SEQ ID NO: 940 - DNA, 24 bp, Homo sapiens
aacaaacaaccaaatacaccttca

SEQ ID NO: 941 - DNA, 24 bp, Homo sapiens
cacacaaaaatactaaacctccca

SEQ ID NO: 942 - DNA, 24 bp, Homo sapiens
cacacacaaataacttcaccctca

SEQ ID NO: 943 - DNA, 27 bp, Homo sapiens
cataaccccttataactaaacaaaaca

SEQ ID NO: 944 - DNA, 20 bp, Homo sapiens
actcctacaaaacccccaca

SEQ ID NO: 945 - DNA, 24 bp, Homo sapiens
acaataaccaccttcctatttcca

SEQ ID NO: 946 - DNA, 23 bp, Homo sapiens
caccacctaaaacctaaaaacca

SEQ ID NO: 947 - DNA, 24 bp, Homo sapiens
ccaaatcttaacaaacaaccaaca

SEQ ID NO: 948 - DNA, 23 bp, Homo sapiens
ctcctctcctcaacactaatcca

SEQ ID NO: 949 - DNA, 24 bp, Homo sapiens
aaaaataacaacctaacccctaca

SEQ ID NO: 950 - DNA, 20 bp, Homo sapiens
cccaaaacctaactccctca

SEQ ID NO: 951 - DNA, 21 bp, Homo sapiens
caccaatcaacaccaacctca

SEQ ID NO: 952 - DNA, 25 bp, Homo sapiens
cacactcaattacttaacaacctca

SEQ ID NO: 953 - DNA, 23 bp, Homo sapiens
atctcaactcatcacaacctcca

SEQ ID NO: 954 - DNA, 21 bp, Homo sapiens
ataactctaccccctccccca

SEQ ID NO: 955 - DNA, 26 bp, Homo sapiens
caaaacacctaaattacctacaacca

SEQ ID NO: 956 - DNA, 24 bp, Homo sapiens
aacttcactttctccctctcttca

SEQ ID NO: 957 - DNA, 25 bp, Homo sapiens
aatcaaacacataactttaacccca

SEQ ID NO: 958 - DNA, 24 bp, Homo sapiens
ttccccactcttattaactttcca

SEQ ID NO: 959 - DNA, 24 bp, Homo sapiens
tcccaccaactctataatcaaaca

SEQ ID NO: 960 - DNA, 25 bp, Homo sapiens
caaattctctaactcacttccttca

SEQ ID NO: 961 - DNA, 24 bp, Homo sapiens
cactaactttttcaaacctttcca

SEQ ID NO: 962 - DNA, 25 bp, Homo sapiens
cacaacatataaacaccacctctca

SEQ ID NO: 963 - DNA, 24 bp, Homo sapiens
ctcccaaatcaactctaacactca

SEQ ID NO: 964 - DNA, 23 bp, Homo sapiens
aaacaccctaacttccaactcca

SEQ ID NO: 965 - DNA, 25 bp, Homo sapiens
ttttcattaataaaaccaaccaaca

SEQ ID NO: 966 - DNA, 24 bp, Homo sapiens
aaaaacctactaactccacccaca

SEQ ID NO: 967 - DNA, 23 bp, Homo sapiens
caacccaacctctatcaaaaaca

SEQ ID NO: 968 - DNA, 27 bp, Homo sapiens
ttccctataacataaccttaaaaaaca

SEQ ID NO: 969 - DNA, 25 bp, Homo sapiens
aacaaatcacaaaatcaaaaattca

SEQ ID NO: 970 - DNA, 25 bp, Homo sapiens
caataactcaactcacaacctctca

SEQ ID NO: 971 - DNA, 24 bp, Homo sapiens
atccttaacctacctcaacctcca

SEQ ID NO: 972 - DNA, 27 bp, Homo sapiens
aaccaacctaaaaactaaaaaataaca

SEQ ID NO: 973 - DNA, 26 bp, Homo sapiens
aaaatcttactatatctcccaaacca

SEQ ID NO: 974 - DNA, 24 bp, Homo sapiens
caaaaatcataccattacactcca

SEQ ID NO: 975 - DNA, 21 bp, Homo sapiens
accattaccaacaatccacca

SEQ ID NO: 976 - DNA, 23 bp, Homo sapiens
aattaaaccaaccttctttccca

SEQ ID NO: 977 - DNA, 21 bp, Homo sapiens
ctcatccactccatctctcca

SEQ ID NO: 978 - DNA, 23 bp, Homo sapiens
aaatcatcctccctcactaacca

SEQ ID NO: 979 - DNA, 23 bp, Homo sapiens
tccccaaacataataaaaccaca

SEQ ID NO: 980 - DNA, 26 bp, Homo sapiens
cacctaactaacacaaacatttctca

SEQ ID NO: 981 - DNA, 24 bp, Homo sapiens
ccaatctctaatatccacctctca

SEQ ID NO: 982 - DNA, 24 bp, Homo sapiens
aaaccaactcaaaacaaacactca

SEQ ID NO: 983 - DNA, 25 bp, Homo sapiens
cattcattaaaatacctacccctca

SEQ ID NO: 984 - DNA, 24 bp, Homo sapiens
accttttcaattactcacaatcca

SEQ ID NO: 985 - DNA, 24 bp, Homo sapiens
aacactttaaccaatcaacaaaca

SEQ ID NO: 986 - DNA, 26 bp, Homo sapiens
ccatttctatacaaaccctacttcca

SEQ ID NO: 987 - DNA, 24 bp, Homo sapiens
caaaaattcaacctcaatcaaaca

SEQ ID NO: 988 - DNA, 26 bp, Homo sapiens
caaaaataaacattctaaccctcaca

SEQ ID NO: 989 - DNA, 23 bp, Homo sapiens
acctcccactcaatcaaactaca

SEQ ID NO: 990 - DNA, 26 bp, Homo sapiens
tccactacaatattactccaaatcca

SEQ ID NO: 991 - DNA, 23 bp, Homo sapiens
aacaaaaactcccttaaacccca

SEQ ID NO: 992 - DNA, 25 bp, Homo sapiens
cccaaatccaacattaataaaaaca

SEQ ID NO: 993 - DNA, 21 bp, Homo sapiens
cctaacttaaatccccctcca

SEQ ID NO: 994 - DNA, 28 bp, Homo sapiens
ccaaaacaccctatataaaacaactaca

SEQ ID NO: 995 - DNA, 22 bp, Homo sapiens
ccaaaaccaatcaaaacaaaca

SEQ ID NO: 996 - DNA, 25 bp, Homo sapiens
ctccaactttaacttcttcaaaaca

SEQ ID NO: 997 - DNA, 26 bp, Homo sapiens
ctcttctccactctaaaacaataaca

SEQ ID NO: 998 - DNA, 25 bp, Homo sapiens
aaaaaattattttctccacatttca

SEQ ID NO: 999 - DNA, 25 bp, Homo sapiens
tttacatcattttaccaatttcaca

SEQ ID NO: 1000 - DNA, 20 bp, Homo sapiens
caatcattcccaaccactca

SEQ ID NO: 1001 - DNA, 24 bp, Homo sapiens
ccccaacaccactataatatacca

SEQ ID NO: 1002 - DNA, 24 bp, Homo sapiens
aaaaccccattttctatcaaaaca

SEQ ID NO: 1003 - DNA, 24 bp, Homo sapiens
aaaatataaacaatcacaacccca

SEQ ID NO: 1004 - DNA, 25 bp, Homo sapiens
tttctacactttcctaaaattccca

SEQ ID NO: 1005 - DNA, 24 bp, Homo sapiens
aaacatccctcaattaaataacca

SEQ ID NO: 1006 - DNA, 21 bp, Homo sapiens
cctctcttctaaaccctccca

SEQ ID NO: 1007 - DNA, 25 bp, Homo sapiens
aaatccaaaaaataaccaaaaaaca

SEQ ID NO: 1008 - DNA, 25 bp, Homo sapiens
caaacaccaaacaaaacaaactaca

SEQ ID NO: 1009 - DNA, 24 bp, Homo sapiens
caatctcaactcactacaacctca

SEQ ID NO: 1010 - DNA, 28 bp, Homo sapiens
caaaatcacactactacactcctaaaca

SEQ ID NO: 1011 - DNA, 23 bp, Homo sapiens
acaccaatttaaaaaccaaccca

SEQ ID NO: 1012 - DNA, 25 bp, Homo sapiens
cacacaatacaaaacctacaaacca

SEQ ID NO: 1013 - DNA, 24 bp, Homo sapiens
cccacaataacatcaaccaataca

SEQ ID NO: 1014 - DNA, 25 bp, Homo sapiens
caaaacacttcttcaaattcctaca

SEQ ID NO: 1015 - DNA, 26 bp, Homo sapiens
aaatcaacaatcaaataaaccaatca

SEQ ID NO: 1016 - DNA, 26 bp, Homo sapiens
tcctactactcccactaattcctaca

SEQ ID NO: 1017 - DNA, 25 bp, Homo sapiens
cctaaaaaatcaatcaaattttcca

SEQ ID NO: 1018 - DNA, 26 bp, Homo sapiens
tctcctcctacaactccataactaca

SEQ ID NO: 1019 - DNA, 27 bp, Homo sapiens
aactctaacattacaaacctcacttca

SEQ ID NO: 1020 - DNA, 26 bp, Homo sapiens
caacttccacatctaaactacttcca

SEQ ID NO: 1021 - DNA, 27 bp, Homo sapiens
ttcctactactacttctaccactccca

SEQ ID NO: 1022 - DNA, 22 bp, Homo sapiens
acactcaacaaccatacccaca

SEQ ID NO: 1023 - DNA, 24 bp, Homo sapiens
aacttaaacctcaaacaaatcaca

SEQ ID NO: 1024 - DNA, 23 bp, Homo sapiens
atcttccaacaaatttcctacca

SEQ ID NO: 1025 - DNA, 26 bp, Homo sapiens
caatacattccttttcatcaaaatca

SEQ ID NO: 1026 - DNA, 24 bp, Homo sapiens
cattaccaacaacttttaaatcca

SEQ ID NO: 1027 - DNA, 23 bp, Homo sapiens
aaaacaaactccacctaacacca

SEQ ID NO: 1028 - DNA, 25 bp, Homo sapiens
ataccaaacacctataccttaacca

SEQ ID NO: 1029 - DNA, 25 bp, Homo sapiens
ctataaaacaaccaaaacacaaaca

SEQ ID NO: 1030 - DNA, 24 bp, Homo sapiens
tttcaaaaactaacaaaccaaaca

SEQ ID NO: 1031 - DNA, 17 bp, Homo sapiens
atcccctcaacccaaca

SEQ ID NO: 1032 - DNA, 26 bp, Homo sapiens
ccaccttaacacatatcctaatctca

SEQ ID NO: 1033 - DNA, 26 bp, Homo sapiens
caaaaatccacattaaaccaaataca

SEQ ID NO: 1034 - DNA, 24 bp, Homo sapiens
caaaaaccaaaacttaaatcctca

SEQ ID NO: 1035 - DNA, 19 bp, Homo sapiens
acaaaacccaaaaacccca

SEQ ID NO: 1036 - DNA, 28 bp, Homo sapiens
actaacctatcaacctctaatacactca

SEQ ID NO: 1037 - DNA, 25 bp, Homo sapiens
tccaaacacaactcaatttaaaaca

SEQ ID NO: 1038 - DNA, 23 bp, Homo sapiens
aacccctaaatccatctttcaca

SEQ ID NO: 1039 - DNA, 24 bp, Homo sapiens
ccccattctatctaccctatctca

SEQ ID NO: 1040 - DNA, 23 bp, Homo sapiens
cctaaaaaacacctaaacccaca

SEQ ID NO: 1041 - DNA, 24 bp, Homo sapiens
acaaacaaaaataacattttccca

SEQ ID NO: 1042 - DNA, 24 bp, Homo sapiens
tcacaaccaaaacaaactacaaca

SEQ ID NO: 1043 - DNA, 25 bp, Homo sapiens
tcatcttacaatcacataaccatca

SEQ ID NO: 1044 - DNA, 28 bp, Homo sapiens
catacactaactccaaaacaactaaaca

SEQ ID NO: 1045 - DNA, 23 bp, Homo sapiens
actctaccccaaccacaactaca

SEQ ID NO: 1046 - DNA, 23 bp, Homo sapiens
acccaacaaacttttccttttca

SEQ ID NO: 1047 - DNA, 25 bp, Homo sapiens
cacacaattaatatcctaccctcca

SEQ ID NO: 1048 - DNA, 21 bp, Homo sapiens
tccccaatacaaaccacaaca

SEQ ID NO: 1049 - DNA, 26 bp, Homo sapiens
aaccaaaacactactcctaactcaca

SEQ ID NO: 1050 - DNA, 23 bp, Homo sapiens
cataaaccaaaaacaacatccca

SEQ ID NO: 1051 - DNA, 23 bp, Homo sapiens
aaacaaacacaaacaaaacacca

SEQ ID NO: 1052 - DNA, 28 bp, Homo sapiens
caataccctataaacctaatcattaaca

SEQ ID NO: 1053 - DNA, 23 bp, Homo sapiens
tacatctaccctccacatctcca

SEQ ID NO: 1054 - DNA, 28 bp, Homo sapiens
cccaaaacactcaataaacaataataca

SEQ ID NO: 1055 - DNA, 23 bp, Homo sapiens
aacccctaactacttcaacctca

SEQ ID NO: 1056 - DNA, 23 bp, Homo sapiens
acaaatcttaaaccctaatccca

SEQ ID NO: 1057 - DNA, 22 bp, Homo sapiens
cccttctatataaacctcccca

SEQ ID NO: 1058 - DNA, 19 bp, Homo sapiens
aaacaactatccccaccca

SEQ ID NO: 1059 - DNA, 24 bp, Homo sapiens
tccttactaccacacatttaacca

SEQ ID NO: 1060 - DNA, 24 bp, Homo sapiens
ttcaaatcttatcaaacaccctca

SEQ ID NO: 1061 - DNA, 24 bp, Homo sapiens
caaacccaacctatatccaaatca

SEQ ID NO: 1062 - DNA, 25 bp, Homo sapiens
aacaatactaaaaacacccctctca

SEQ ID NO: 1063 - DNA, 24 bp, Homo sapiens
ccctttaaccactaacctaatcca

SEQ ID NO: 1064 - DNA, 24 bp, Homo sapiens
ccacccaaaacaactacactatca

SEQ ID NO: 1065 - DNA, 23 bp, Homo sapiens
aaattaccccacaacctaaacca

SEQ ID NO: 1066 - DNA, 23 bp, Homo sapiens
aaccacacaaatcccacttatca

SEQ ID NO: 1067 - DNA, 23 bp, Homo sapiens
aacaccaaacaaaacaaaaacca

SEQ ID NO: 1068 - DNA, 26 bp, Homo sapiens
cacacaacacattacaaacaaataca

SEQ ID NO: 1069 - DNA, 27 bp, Homo sapiens
cacactctctcacttttataaaaaaca

SEQ ID NO: 1070 - DNA, 25 bp, Homo sapiens
aaattacacaaaccattctaaccca

SEQ ID NO: 1071 - DNA, 26 bp, Homo sapiens
aaacaaaaaactatccaccttacaca

SEQ ID NO: 1072 - DNA, 23 bp, Homo sapiens
actcactcttatccccacctaca

SEQ ID NO: 1073 - DNA, 22 bp, Homo sapiens
cccaccaacatcctaaatacca

SEQ ID NO: 1074 - DNA, 21 bp, Homo sapiens
aactacacaacccaaaccaca

SEQ ID NO: 1075 - DNA, 19 bp, Homo sapiens
acctcaaatcctccaacca

SEQ ID NO: 1076 - DNA, 23 bp, Homo sapiens
acccaaatacaactacccaaaca

SEQ ID NO: 1077 - DNA, 25 bp, Homo sapiens
ccaaaccaactcaaatctacctaca

SEQ ID NO: 1078 - DNA, 24 bp, Homo sapiens
aaactctaacaacccatcctaaca

SEQ ID NO: 1079 - DNA, 24 bp, Homo sapiens
acttacatcctctaaatttcccca

SEQ ID NO: 1080 - DNA, 23 bp, Homo sapiens
aacaacttaatccccctaaccca

SEQ ID NO: 1081 - DNA, 26 bp, Homo sapiens
aatataacaaccaatcctactctcca

SEQ ID NO: 1082 - DNA, 23 bp, Homo sapiens
tcctaaaaacccttaaccctcca

SEQ ID NO: 1083 - DNA, 27 bp, Homo sapiens
aactactattctctcctatttccctca

SEQ ID NO: 1084 - DNA, 24 bp, Homo sapiens
ataaaactttctccccaaaaaaca

SEQ ID NO: 1085 - DNA, 24 bp, Homo sapiens
aaattcctccacttaaactaccca

SEQ ID NO: 1086 - DNA, 23 bp, Homo sapiens
aaactcaaaaccctccaaaatca

SEQ ID NO: 1087 - DNA, 25 bp, Homo sapiens
ctctaactaaactcaacaacctcca

SEQ ID NO: 1088 - DNA, 26 bp, Homo sapiens
aaaaccttaactaacacaaaaccaca

SEQ ID NO: 1089 - DNA, 25 bp, Homo sapiens
ccaaatctcactcctaaattcaaca

SEQ ID NO: 1090 - DNA, 23 bp, Homo sapiens
aaaaaacctctacccaaaaacca

SEQ ID NO: 1091 - DNA, 24 bp, Homo sapiens
aaaatacaaactacttcaccccca

SEQ ID NO: 1092 - DNA, 23 bp, Homo sapiens
ccaacccaaaacctactcctaca

SEQ ID NO: 1093 - DNA, 26 bp, Homo sapiens
ctctaaatttaaattaccctcctcca

SEQ ID NO: 1094 - DNA, 25 bp, Homo sapiens
aacccctctaattatctctttacca

SEQ ID NO: 1095 - DNA, 24 bp, Homo sapiens
acaacctatccaaatcacaaaaca

SEQ ID NO: 1096 - DNA, 25 bp, Homo sapiens
cttacaaatctcccaaataaaatca

SEQ ID NO: 1097 - DNA, 18 bp, Homo sapiens
acaaacactcccctccca

SEQ ID NO: 1098 - DNA, 24 bp, Homo sapiens
caaaacctcaatctaaaaaccaca

SEQ ID NO: 1099 - DNA, 19 bp, Homo sapiens
acccctccccaaataatca

SEQ ID NO: 1100 - DNA, 28 bp, Homo sapiens
catattatatcctaccatttaataccca

SEQ ID NO: 1101 - DNA, 25 bp, Homo sapiens
ttaaaactcaacctcacaaatcaca

SEQ ID NO: 1102 - DNA, 25 bp, Homo sapiens
ccaaaaccaataactttacaaaaca

SEQ ID NO: 1103 - DNA, 25 bp, Homo sapiens
aaacaaataaaataccacctccaca

SEQ ID NO: 1104 - DNA, 25 bp, Homo sapiens
aattcaaaaaaccacaaaaatttca

SEQ ID NO: 1105 - DNA, 26 bp, Homo sapiens
ttaaaacacctaaacttaaaacctca

SEQ ID NO: 1106 - DNA, 23 bp, Homo sapiens
ataaaaaccaacccaaatttcca

SEQ ID NO: 1107 - DNA, 23 bp, Homo sapiens
caccacatacacactacacacca

SEQ ID NO: 1108 - DNA, 23 bp, Homo sapiens
cacacacacaaaccacatacaca

SEQ ID NO: 1109 - DNA, 19 bp, Homo sapiens
aaaacaaacaccaacccca

SEQ ID NO: 1110 - DNA, 26 bp, Homo sapiens
caaacatacatacaacaatcctctca

SEQ ID NO: 1111 - DNA, 25 bp, Homo sapiens
caaactcctaaactcaaacaatcca

SEQ ID NO: 1112 - DNA, 24 bp, Homo sapiens
aacaatactcaaaaaccaaaacca

SEQ ID NO: 1113 - DNA, 25 bp, Homo sapiens
cacactccctactaaacctaaatca

SEQ ID NO: 1114 - DNA, 23 bp, Homo sapiens
ccaacctcaactcacaaaataca

SEQ ID NO: 1115 - DNA, 24 bp, Homo sapiens
ataacatcacaacccaaataacca

SEQ ID NO: 1116 - DNA, 24 bp, Homo sapiens
ataccacctcacccctaaactaca

SEQ ID NO: 1117 - DNA, 26 bp, Homo sapiens
caataccaaccaactaaactaaacca

SEQ ID NO: 1118 - DNA, 24 bp, Homo sapiens
caacaaataaccaaacaaacacca

SEQ ID NO: 1119 - DNA, 24 bp, Homo sapiens
accaaaacaaaaacaaaaacaaca

SEQ ID NO: 1120 - DNA, 25 bp, Homo sapiens
tccaaaccaacttatctatcctaca

SEQ ID NO: 1121 - DNA, 25 bp, Homo sapiens
ttacaaacctaaaattcctctttca

SEQ ID NO: 1122 - DNA, 23 bp, Homo sapiens
aaacaaaaactacaactctccca

SEQ ID NO: 1123 - DNA, 24 bp, Homo sapiens
accaatacactccaaacctaaaca

SEQ ID NO: 1124 - DNA, 27 bp, Homo sapiens
cactaaaattacaaacataaaccacca

SEQ ID NO: 1125 - DNA, 24 bp, Homo sapiens
actcaaaccctctaacctcaaaca

SEQ ID NO: 1126 - DNA, 23 bp, Homo sapiens
atccccaaaactaaaaaacatcc

SEQ ID NO: 1127 - DNA, 23 bp, Homo sapiens
caccaaactaaaatcaaacccct

SEQ ID NO: 1128 - DNA, 26 bp, Homo sapiens
atccctacacaaaaacataacaaaca

SEQ ID NO: 1129 - DNA, 23 bp, Homo sapiens
ctcccctcaactttcttcaaatt

SEQ ID NO: 1130 - DNA, 22 bp, Homo sapiens
cctcccacaccactcaaaaatt

SEQ ID NO: 1131 - DNA, 26 bp, Homo sapiens
aactaccaatcatatttcctaaacca

SEQ ID NO: 1132 - DNA, 24 bp, Homo sapiens
caccactaacactaaactcctcaa

SEQ ID NO: 1133 - DNA, 22 bp, Homo sapiens
ttcccccaaactccctactatc

SEQ ID NO: 1134 - DNA, 23 bp, Homo sapiens
ccctacataaacaacctcaccaa

SEQ ID NO: 1135 - DNA, 23 bp, Homo sapiens
aatctttacccaaacacttccaa

SEQ ID NO: 1136 - DNA, 24 bp, Homo sapiens
ccctctaaaaacatacaaaccaaa

SEQ ID NO: 1137 - DNA, 24 bp, Homo sapiens
aaataaataaaccacaccaccaac

SEQ ID NO: 1138 - DNA, 21 bp, Homo sapiens
ctccaacaccaatccaacaaa

SEQ ID NO: 1139 - DNA, 26 bp, Homo sapiens
ttatcacacacttatttctcacttcc

SEQ ID NO: 1140 - DNA, 19 bp, Homo sapiens
cctcctcatctcctcccac

SEQ ID NO: 1141 - DNA, 24 bp, Homo sapiens
ataaacaaaacaacaacccacaaa

SEQ ID NO: 1142 - DNA, 24 bp, Homo sapiens
aactattccataaacaccaccacc

SEQ ID NO: 1143 - DNA, 25 bp, Homo sapiens
aactaaactacaactcacccaacca

SEQ ID NO: 1144 - DNA, 24 bp, Homo sapiens
tccttcctttccttaccctacttt

SEQ ID NO: 1145 - DNA, 23 bp, Homo sapiens
aaaccaaacctcacttaaaccac

SEQ ID NO: 1146 - DNA, 22 bp, Homo sapiens
atccaaacaaaccacaaactcc

SEQ ID NO: 1147 - DNA, 24 bp, Homo sapiens
ctccctaacccaatctctaacaca

SEQ ID NO: 1148 - DNA, 26 bp, Homo sapiens
aacaaaaaatttctattccttcaacc

SEQ ID NO: 1149 - DNA, 23 bp, Homo sapiens
cctcataacacatcaacccaaac

SEQ ID NO: 1150 - DNA, 25 bp, Homo sapiens
cctaacccaactctaaaacaaacaa

SEQ ID NO: 1151 - DNA, 24 bp, Homo sapiens
caacccaaaactattacaaaccaa

SEQ ID NO: 1152 - DNA, 23 bp, Homo sapiens
ctcaatccacaaactctccactt

SEQ ID NO: 1153 - DNA, 23 bp, Homo sapiens
attataatcttcccacctcccac

SEQ ID NO: 1154 - DNA, 23 bp, Homo sapiens
caaacaaaacacaacacccatta

SEQ ID NO: 1155 - DNA, 24 bp, Homo sapiens
aaaaaacctacaaactcaaaccca

SEQ ID NO: 1156 - DNA, 25 bp, Homo sapiens
aaacatcaaccctacaacctaaaca

SEQ ID NO: 1157 - DNA, 23 bp, Homo sapiens
acaaaccaacctaaactcccact

SEQ ID NO: 1158 - DNA, 23 bp, Homo sapiens
aaaacactaattcctcaactccc

SEQ ID NO: 1159 - DNA, 24 bp, Homo sapiens
aaatcctaactctacaccctcctc

SEQ ID NO: 1160 - DNA, 28 bp, Homo sapiens
caaacaaattacttacccttacttatca

SEQ ID NO: 1161 - DNA, 28 bp, Homo sapiens
aacaaatcttacacctcttctacatctc

SEQ ID NO: 1162 - DNA, 27 bp, Homo sapiens
taaacaacaacctctcaaaatacaaaa

SEQ ID NO: 1163 - DNA, 27 bp, Homo sapiens
aaaacacttaactctttctatccctcc

SEQ ID NO: 1164 - DNA, 25 bp, Homo sapiens
ctctttccttttacatcatccaaaa

SEQ ID NO: 1165 - DNA, 23 bp, Homo sapiens
acccatctcaactttcatcatca

SEQ ID NO: 1166 - DNA, 24 bp, Homo sapiens
ccctacatttccctctacactaaa

SEQ ID NO: 1167 - DNA, 23 bp, Homo sapiens
atatcacctcatccaactccctt

SEQ ID NO: 1168 - DNA, 21 bp, Homo sapiens
actcctaaattccatccccac

SEQ ID NO: 1169 - DNA, 26 bp, Homo sapiens
aacatccctaccctctctatacaaca

SEQ ID NO: 1170 - DNA, 24 bp, Homo sapiens
ccttctctaaatccacaaaatcac

SEQ ID NO: 1171 - DNA, 23 bp, Homo sapiens
aaacaacttcaaaaccccctacc

SEQ ID NO: 1172 - DNA, 23 bp, Homo sapiens
ccacaaaaccacaacacatctaa

SEQ ID NO: 1173 - DNA, 23 bp, Homo sapiens
tctaaatccccctcctctaatca

SEQ ID NO: 1174 - DNA, 27 bp, Homo sapiens
acactactccccactttatttatttaa

SEQ ID NO: 1175 - DNA, 24 bp, Homo sapiens
aattatcctcctacaccaacctca

SEQ ID NO: 1176 - DNA, 23 bp, Homo sapiens
taaccaatcaccctcttcctttc

SEQ ID NO: 1177 - DNA, 23 bp, Homo sapiens
ataattttcctccttcttccctc

SEQ ID NO: 1178 - DNA, 24 bp, Homo sapiens
ttttcaccttacaatcacccttaa

SEQ ID NO: 1179 - DNA, 25 bp, Homo sapiens
ccacactacaccaacacttatttct

SEQ ID NO: 1180 - DNA, 24 bp, Homo sapiens
aacccaatctatccctataacaaa

SEQ ID NO: 1181 - DNA, 27 bp, Homo sapiens
aaactattacatttactctccacctcc

SEQ ID NO: 1182 - DNA, 24 bp, Homo sapiens
acacccaaatatttcctaacaaaa

SEQ ID NO: 1183 - DNA, 18 bp, Homo sapiens
ccaaccattccaccctca

SEQ ID NO: 1184 - DNA, 22 bp, Homo sapiens
aacccaaccccctcttcattat

SEQ ID NO: 1185 - DNA, 23 bp, Homo sapiens
aacaataaccacacaatcccaac

SEQ ID NO: 1186 - DNA, 23 bp, Homo sapiens
ccaccaaacatacaattccaact

SEQ ID NO: 1187 - DNA, 24 bp, Homo sapiens
attctatccctatcctaatcaccc

SEQ ID NO: 1188 - DNA, 25 bp, Homo sapiens
actcaaacttctctccaaaacaaaa

SEQ ID NO: 1189 - DNA, 23 bp, Homo sapiens
ccactttacaaaacatcaccacc

SEQ ID NO: 1190 - DNA, 23 bp, Homo sapiens
aaacaaacaaaaattcccaaaaa

SEQ ID NO: 1191 - DNA, 25 bp, Homo sapiens
tacccactaaaaactacaacttccc

SEQ ID NO: 1192 - DNA, 22 bp, Homo sapiens
taaaacaacatctcctccccaa

SEQ ID NO: 1193 - DNA, 23 bp, Homo sapiens
taataacatcaccccaaaatcaa

SEQ ID NO: 1194 - DNA, 26 bp, Homo sapiens
atatacaaactccaaaatcacaacaa

SEQ ID NO: 1195 - DNA, 23 bp, Homo sapiens
aaactccaaaacacccctctaca

SEQ ID NO: 1196 - DNA, 25 bp, Homo sapiens
aaataaattcctcacacctcacaac

SEQ ID NO: 1197 - DNA, 24 bp, Homo sapiens
cactacccaatcatcatatcaaaa

SEQ ID NO: 1198 - DNA, 26 bp, Homo sapiens
tattattaaaacctcaatttcccaaa

SEQ ID NO: 1199 - DNA, 21 bp, Homo sapiens
cacccctttccactactcctt

SEQ ID NO: 1200 - DNA, 27 bp, Homo sapiens
ttcatcctattaaaaataccacaaatt

SEQ ID NO: 1201 - DNA, 22 bp, Homo sapiens
cctaattttaaaccctcacccc

SEQ ID NO: 1202 - DNA, 23 bp, Homo sapiens
caccctcatacttccaaaaccta

SEQ ID NO: 1203 - DNA, 23 bp, Homo sapiens
cttcctaatcctaccctacccta

SEQ ID NO: 1204 - DNA, 25 bp, Homo sapiens
cccactacaaaatctctcctctatc

SEQ ID NO: 1205 - DNA, 23 bp, Homo sapiens
cttaaaacacaaaccactcccac

SEQ ID NO: 1206 - DNA, 25 bp, Homo sapiens
cctaatttctacaaccactctaccc

SEQ ID NO: 1207 - DNA, 23 bp, Homo sapiens
tccctctttcacaatctcactcc

SEQ ID NO: 1208 - DNA, 24 bp, Homo sapiens
ttatctcaaactcacccacttcaa

SEQ ID NO: 1209 - DNA, 23 bp, Homo sapiens
acaaatcccataacccacctaaa

SEQ ID NO: 1210 - DNA, 24 bp, Homo sapiens
aaaacaatacctccaaaactcacc

SEQ ID NO: 1211 - DNA, 24 bp, Homo sapiens
accttctctaaaccaacaacacaa

SEQ ID NO: 1212 - DNA, 23 bp, Homo sapiens
aactcctacatttccccctaaca

SEQ ID NO: 1213 - DNA, 27 bp, Homo sapiens
ccaatttcctatacataacatcacaca

SEQ ID NO: 1214 - DNA, 22 bp, Homo sapiens
cactaactacaaccacctccca

SEQ ID NO: 1215 - DNA, 23 bp, Homo sapiens
aacaaaaactccaaaaaacaacc

SEQ ID NO: 1216 - DNA, 24 bp, Homo sapiens
aatcaacaaaacaaaccaaaacaa

SEQ ID NO: 1217 - DNA, 23 bp, Homo sapiens
ctaaaccaattccacaaacctca

SEQ ID NO: 1218 - DNA, 23 bp, Homo sapiens
acaaccccaccttctctacctac

SEQ ID NO: 1219 - DNA, 24 bp, Homo sapiens
aaaatcccaaaatcttacattccc

SEQ ID NO: 1220 - DNA, 24 bp, Homo sapiens
aaattcctcaattattcctccacc

SEQ ID NO: 1221 - DNA, 23 bp, Homo sapiens
aacaactcatccaaataaccaaa

SEQ ID NO: 1222 - DNA, 24 bp, Homo sapiens
ataacccttcaaccaatacaaacc

SEQ ID NO: 1223 - DNA, 25 bp, Homo sapiens
aaatacacaaactacctcaaaccca

SEQ ID NO: 1224 - DNA, 23 bp, Homo sapiens
ttacacacccacaaactatccct

SEQ ID NO: 1225 - DNA, 25 bp, Homo sapiens
aatccaccaatcaataaacaacttc

SEQ ID NO: 1226 - DNA, 23 bp, Homo sapiens
ccaaacctccacaaatcttcctt

SEQ ID NO: 1227 - DNA, 22 bp, Homo sapiens
ccaccaaaacccaactctaaaa

SEQ ID NO: 1228 - DNA, 25 bp, Homo sapiens
tattcaaatacccaaaactaaaccc

SEQ ID NO: 1229 - DNA, 28 bp, Homo sapiens
cacacttaaatctacctctattacctcc

SEQ ID NO: 1230 - DNA, 20 bp, Homo sapiens
atcctaacccctacaacccc

SEQ ID NO: 1231 - DNA, 29 bp, Homo sapiens
aaaatacctatccctaaataaaaatcatt

SEQ ID NO: 1232 - DNA, 23 bp, Homo sapiens
ccttccttctaacccactaaaaa

SEQ ID NO: 1233 - DNA, 29 bp, Homo sapiens
aaacatactttcttaaaccttaaacaaaa

SEQ ID NO: 1234 - DNA, 23 bp, Homo sapiens
cactacactccaacccaaacaac

SEQ ID NO: 1235 - DNA, 27 bp, Homo sapiens
accttattacaacatcaacacattaca

SEQ ID NO: 1236 - DNA, 25 bp, Homo sapiens
attacccattaaatcttaattcccc

SEQ ID NO: 1237 - DNA, 21 bp, Homo sapiens
accttacacctcaacccattt

SEQ ID NO: 1238 - DNA, 27 bp, Homo sapiens
ctatatataaaacaaccccatttctca

SEQ ID NO: 1239 - DNA, 24 bp, Homo sapiens
atactcacaacctctcacacaaaa

SEQ ID NO: 1240 - DNA, 26 bp, Homo sapiens
ctttctcactttaacaccaactaccc

SEQ ID NO: 1241 - DNA, 23 bp, Homo sapiens
aaacaaaacaatcccaccctaca

SEQ ID NO: 1242 - DNA, 29 bp, Homo sapiens
attaaacacaaactacaataaccttcaca

SEQ ID NO: 1243 - DNA, 23 bp, Homo sapiens
caaataaccaacaaaaacctcca

SEQ ID NO: 1244 - DNA, 25 bp, Homo sapiens
cacctaacacaacacctaataccct

SEQ ID NO: 1245 - DNA, 26 bp, Homo sapiens
acaaaccaacccttctaatatactcc

SEQ ID NO: 1246 - DNA, 23 bp, Homo sapiens
aacttcctaccccacccaataaa

SEQ ID NO: 1247 - DNA, 23 bp, Homo sapiens
aactctcctcaactccttcccac

SEQ ID NO: 1248 - DNA, 22 bp, Homo sapiens
aaccccaatctccacaataaaa

SEQ ID NO: 1249 - DNA, 24 bp, Homo sapiens
taccctatacccacaatattccaa

SEQ ID NO: 1250 - DNA, 23 bp, Homo sapiens
aactaaaaaactccaaaaccccc

SEQ ID NO: 1251 - DNA, 25 bp, Homo sapiens
ctctacaatccatttatttacccac

SEQ ID NO: 1252 - DNA, 22 bp, Homo sapiens
acccaaccacattaacaaacca

SEQ ID NO: 1253 - DNA, 24 bp, Homo sapiens
tactaacaaactcctcactccaaa

SEQ ID NO: 1254 - DNA, 23 bp, Homo sapiens
aaacataaaaccaaaaacacccc

SEQ ID NO: 1255 - DNA, 28 bp, Homo sapiens
tccttactcaaatctttaccaaataacc

SEQ ID NO: 1256 - DNA, 23 bp, Homo sapiens
ccaatctaaccttccaaacacat

SEQ ID NO: 1257 - DNA, 23 bp, Homo sapiens
attaaaccttctctcatcacccc

SEQ ID NO: 1258 - DNA, 23 bp, Homo sapiens
ataaacacaacaccctacacacc

SEQ ID NO: 1259 - DNA, 26 bp, Homo sapiens
aacacacacaatacaatcacacttaa

SEQ ID NO: 1260 - DNA, 21 bp, Homo sapiens
acaactcccaaaacaaaaccc

SEQ ID NO: 1261 - DNA, 23 bp, Homo sapiens
ctcctcaccttccctttatatcc

SEQ ID NO: 1262 - DNA, 23 bp, Homo sapiens
taacaacaaccacaaacacaaaa

SEQ ID NO: 1263 - DNA, 25 bp, Homo sapiens
aactcaatttcttccacctaaaaac

SEQ ID NO: 1264 - DNA, 23 bp, Homo sapiens
ccaacttccaaccaaaaactaca

SEQ ID NO: 1265 - DNA, 27 bp, Homo sapiens
ataacactcaaatccaactaaacaaca

SEQ ID NO: 1266 - DNA, 26 bp, Homo sapiens
acaatcaacaactaacaacctaacaa

SEQ ID NO: 1267 - DNA, 26 bp, Homo sapiens
accttataaaaaacacaaaccacacc

SEQ ID NO: 1268 - DNA, 24 bp, Homo sapiens
tatatccaacaaacatcaaccacc

SEQ ID NO: 1269 - DNA, 22 bp, Homo sapiens
cctaaactacacttttccccct

SEQ ID NO: 1270 - DNA, 25 bp, Homo sapiens
acaattacttcaaaacatctccaaa

SEQ ID NO: 1271 - DNA, 21 bp, Homo sapiens
cctcccaaaccattcaaaaac

SEQ ID NO: 1272 - DNA, 22 bp, Homo sapiens
aaccaaaacaatctcctccaac

SEQ ID NO: 1273 - DNA, 22 bp, Homo sapiens
accccttaaaattccaaaaacc

SEQ ID NO: 1274 - DNA, 26 bp, Homo sapiens
ccacctcctcaacttactctaacttc

SEQ ID NO: 1275 - DNA, 23 bp, Homo sapiens
accacaaacactccataaacaca

SEQ ID NO: 1276 - DNA, 26 bp, Homo sapiens
ttcacttaactaacaacacaacctcc

SEQ ID NO: 1277 - DNA, 27 bp, Homo sapiens
atacaaaatatttaaaaaccctcacaa

SEQ ID NO: 1278 - DNA, 25 bp, Homo sapiens
taactcctctcattccaatacctaa

SEQ ID NO: 1279 - DNA, 23 bp, Homo sapiens
aatacccccacaactctaaacct

SEQ ID NO: 1280 - DNA, 21 bp, Homo sapiens
ccaaaaaaccaatcaaaacca

SEQ ID NO: 1281 - DNA, 24 bp, Homo sapiens
acacaaaactccaaattcaatttc

SEQ ID NO: 1282 - DNA, 27 bp, Homo sapiens
aaacacatacataacaacaacaacaac

SEQ ID NO: 1283 - DNA, 23 bp, Homo sapiens
aatcccatttcaaaatttcctct

SEQ ID NO: 1284 - DNA, 27 bp, Homo sapiens
attatcaaacaacaaacatctacctct

SEQ ID NO: 1285 - DNA, 23 bp, Homo sapiens
ttcccctttacaaactaccaacc

SEQ ID NO: 1286 - DNA, 23 bp, Homo sapiens
acaaccaaaaataaaaccaaacc

SEQ ID NO: 1287 - DNA, 24 bp, Homo sapiens
aatcccaatcccttaattaaactt

SEQ ID NO: 1288 - DNA, 22 bp, Homo sapiens
ccccatttctcactcccattaa

SEQ ID NO: 1289 - DNA, 18 bp, Homo sapiens
aaacccccaacctacccc

SEQ ID NO: 1290 - DNA, 22 bp, Homo sapiens
aatacaaatcacctctcccaaa

SEQ ID NO: 1291 - DNA, 18 bp, Homo sapiens
accccacccaacttcaaa

SEQ ID NO: 1292 - DNA, 25 bp, Homo sapiens
attcaaacaattctcctacctcaac

SEQ ID NO: 1293 - DNA, 24 bp, Homo sapiens
aacttacccttccaattaaccctc

SEQ ID NO: 1294 - DNA, 24 bp, Homo sapiens
actcctaactcaaataatccaccc

SEQ ID NO: 1295 - DNA, 23 bp, Homo sapiens
attaaatccaaacctccttcacc

SEQ ID NO: 1296 - DNA, 22 bp, Homo sapiens
aatactaccccctccaaatccc

SEQ ID NO: 1297 - DNA, 26 bp, Homo sapiens
ctacttaccacacttccaaactacca

SEQ ID NO: 1298 - DNA, 22 bp, Homo sapiens
ccccatacccctcaaaacctat

SEQ ID NO: 1299 - DNA, 24 bp, Homo sapiens
tttcttctccatcacaaaaacaaa

SEQ ID NO: 1300 - DNA, 26 bp, Homo sapiens
ttcctccaaataaactcataaatttc

SEQ ID NO: 1301 - DNA, 23 bp, Homo sapiens
ccccaaaaactctatccaaaaac

SEQ ID NO: 1302 - DNA, 24 bp, Homo sapiens
accaaacttcactaaacatccaca

SEQ ID NO: 1303 - DNA, 25 bp, Homo sapiens
acataacctcactttcctactccct

SEQ ID NO: 1304 - DNA, 24 bp, Homo sapiens
ccctacccaaactcactacttacc

SEQ ID NO: 1305 - DNA, 24 bp, Homo sapiens
aatttcccctaatctcttcattca

SEQ ID NO: 1306 - DNA, 24 bp, Homo sapiens
tacacttccaaaccttcaacaaac

SEQ ID NO: 1307 - DNA, 24 bp, Homo sapiens
caaacacacacatacacacacaca

SEQ ID NO: 1308 - DNA, 28 bp, Homo sapiens
aacattataattccaaccaaaaatactc

SEQ ID NO: 1309 - DNA, 24 bp, Homo sapiens
taaccctactcttataacctccca

SEQ ID NO: 1310 - DNA, 26 bp, Homo sapiens
tcttccttcaactataacttacacca

SEQ ID NO: 1311 - DNA, 26 bp, Homo sapiens
actcaacctcataataacacctaaca

SEQ ID NO: 1312 - DNA, 28 bp, Homo sapiens
caatttctcacctatacatttcctataa

SEQ ID NO: 1313 - DNA, 22 bp, Homo sapiens
tcctcccctactacccctcaac

SEQ ID NO: 1314 - DNA, 22 bp, Homo sapiens
cccaaactaacacatccacaac

SEQ ID NO: 1315 - DNA, 24 bp, Homo sapiens
caaaacaatactaattccaacccc

SEQ ID NO: 1316 - DNA, 23 bp, Homo sapiens
ccattctaaacaaaaacccaaca

SEQ ID NO: 1317 - DNA, 23 bp, Homo sapiens
ctcaacaacctcaaaaactaccc

SEQ ID NO: 1318 - DNA, 29 bp, Homo sapiens
aaaaactctataaaaataacaacaacctc

SEQ ID NO: 1319 - DNA, 22 bp, Homo sapiens
cccacccaactataaaaaacca

SEQ ID NO: 1320 - DNA, 24 bp, Homo sapiens
ccaacaaacaacactacaaaacaa

SEQ ID NO: 1321 - DNA, 23 bp, Homo sapiens
cactacaacccaaaacaatccaa

SEQ ID NO: 1322 - DNA, 23 bp, Homo sapiens
aactcacaaatccctttcctaac

SEQ ID NO: 1323 - DNA, 25 bp, Homo sapiens
aaaaaacaaactccaaacaaaaaca

SEQ ID NO: 1324 - DNA, 21 bp, Homo sapiens
tccccaacaatctctattccc

SEQ ID NO: 1325 - DNA, 19 bp, Homo sapiens
cacacccacttcctacccc

SEQ ID NO: 1326 - DNA, 23 bp, Homo sapiens
cttcacaaacctaaccaatcaat

SEQ ID NO: 1327 - DNA, 23 bp, Homo sapiens
cactaaaccaccccaaataaaac

SEQ ID NO: 1328 - DNA, 23 bp, Homo sapiens
ctcccaaattcaaacaattctcc

SEQ ID NO: 1329 - DNA, 27 bp, Homo sapiens
aatccaaaataaattccctaaactacc

SEQ ID NO: 1330 - DNA, 24 bp, Homo sapiens
tctcacacttttcctcaaaaaatc

SEQ ID NO: 1331 - DNA, 23 bp, Homo sapiens
acaaacctaaatacatccctcct

SEQ ID NO: 1332 - DNA, 25 bp, Homo sapiens
accttcattcaaacacttaaaccac

SEQ ID NO: 1333 - DNA, 23 bp, Homo sapiens
aactacttcctctccctccacaa

SEQ ID NO: 1334 - DNA, 25 bp, Homo sapiens
ttcaaaaatcaaccaatcataatcc

SEQ ID NO: 1335 - DNA, 24 bp, Homo sapiens
tcacaaacaaattaacttcccaaa

SEQ ID NO: 1336 - DNA, 28 bp, Homo sapiens
ttaactacttactttcttctctccaccc

SEQ ID NO: 1337 - DNA, 25 bp, Homo sapiens
aaaaccaatttactcacaaacaaaa

SEQ ID NO: 1338 - DNA, 24 bp, Homo sapiens
caactaaacccaacaccaatctaa

SEQ ID NO: 1339 - DNA, 22 bp, Homo sapiens
aaacaatccaaacccaaaaacc

SEQ ID NO: 1340 - DNA, 23 bp, Homo sapiens
aaacacatacacaacaaaccacc

SEQ ID NO: 1341 - DNA, 21 bp, Homo sapiens
tcctaccctcaccctcctaaa

SEQ ID NO: 1342 - DNA, 26 bp, Homo sapiens
attctcaaaataaaaattccctatca

SEQ ID NO: 1343 - DNA, 22 bp, Homo sapiens
cattaccctttctatccccacc

SEQ ID NO: 1344 - DNA, 27 bp, Homo sapiens
aaccaacctaaccaacataataaaacc

SEQ ID NO: 1345 - DNA, 23 bp, Homo sapiens
taaactccaaacacccacctacc

SEQ ID NO: 1346 - DNA, 21 bp, Homo sapiens
accctccaaacctccaactac

SEQ ID NO: 1347 - DNA, 23 bp, Homo sapiens
ctacatctactcatttcctccca

SEQ ID NO: 1348 - DNA, 27 bp, Homo sapiens
aatacaataacataacctctacctccc

SEQ ID NO: 1349 - DNA, 24 bp, Homo sapiens
cctaaaaaacacaacaaaactcca

SEQ ID NO: 1350 - DNA, 24 bp, Homo sapiens
ttaccacactctaaccaatcaaaa

SEQ ID NO: 1351 - DNA, 26 bp, Homo sapiens
ctaccaaattcatttaaaaaccaaaa

SEQ ID NO: 1352 - DNA, 25 bp, Homo sapiens
ttccctttcttcacaatacttaaca

SEQ ID NO: 1353 - DNA, 23 bp, Homo sapiens
ttcaactccaaaatttccaaact

SEQ ID NO: 1354 - DNA, 23 bp, Homo sapiens
ttcatcactcccacaattaacaa

SEQ ID NO: 1355 - DNA, 23 bp, Homo sapiens
aaaacaaccaaaacaaaaccaaa

SEQ ID NO: 1356 - DNA, 22 bp, Homo sapiens
catcaaccaaacattccaaaaa

SEQ ID NO: 1357 - DNA, 22 bp, Homo sapiens
aaaacacatatcctcaccccac

SEQ ID NO: 1358 - DNA, 22 bp, Homo sapiens
accctcactaactctcctccac

SEQ ID NO: 1359 - DNA, 25 bp, Homo sapiens
caatccttaaaccaatcaacataaa

SEQ ID NO: 1360 - DNA, 24 bp, Homo sapiens
caaaacattaaactcctcctcctc

SEQ ID NO: 1361 - DNA, 26 bp, Homo sapiens
taaacaacataacatacaacaccacc

SEQ ID NO: 1362 - DNA, 24 bp, Homo sapiens
tcaactccatttcaatttcacttc

SEQ ID NO: 1363 - DNA, 24 bp, Homo sapiens
aacaatacaacctatcaccaccct

SEQ ID NO: 1364 - DNA, 24 bp, Homo sapiens
tactttaaaccaaaccaaaccaaa

SEQ ID NO: 1365 - DNA, 23 bp, Homo sapiens
aaaaattcaaactcccaaacaca

SEQ ID NO: 1366 - DNA, 23 bp, Homo sapiens
cttcttccaactcttcacctcaa

SEQ ID NO: 1367 - DNA, 25 bp, Homo sapiens
aacacatccctaaaacttaaccaca

SEQ ID NO: 1368 - DNA, 23 bp, Homo sapiens
ttcaccacaaaccttctttcaac

SEQ ID NO: 1369 - DNA, 26 bp, Homo sapiens
acaaacaactaaactcaactaccaaa

SEQ ID NO: 1370 - DNA, 27 bp, Homo sapiens
ctactaaaaaaccaatcaacacactca

SEQ ID NO: 1371 - DNA, 26 bp, Homo sapiens
atcctaatcctcctacccttatattt

SEQ ID NO: 1372 - DNA, 29 bp, Homo sapiens
aaactaactaaacaattaactctcctccc

SEQ ID NO: 1373 - DNA, 26 bp, Homo sapiens
aattctaaacatttcccaactacaaa

SEQ ID NO: 1374 - DNA, 22 bp, Homo sapiens
actacaaccaacaaaccacacc

SEQ ID NO: 1375 - DNA, 23 bp, Homo sapiens
aacccaaaactaccacaaaaaac

SEQ ID NO: 1376 - DNA, 23 bp, Homo sapiens
aatcctaaacctaaacctcccaa

SEQ ID NO: 1377 - DNA, 21 bp, Homo sapiens
accaaatcttcccttatcccc

SEQ ID NO: 1378 - DNA, 23 bp, Homo sapiens
tccttacaacctctaacctcctt

SEQ ID NO: 1379 - DNA, 24 bp, Homo sapiens
cctcttaaaacctacctacccctc

SEQ ID NO: 1380 - DNA, 26 bp, Homo sapiens
aaattacaccaattataaaccaaaca

SEQ ID NO: 1381 - DNA, 23 bp, Homo sapiens
aaaaccctctacccacctattca

SEQ ID NO: 1382 - DNA, 22 bp, Homo sapiens
aaaatcaaaaccaaaacccctc

SEQ ID NO: 1383 - DNA, 26 bp, Homo sapiens
ccataacaactacacaactcctaacc

SEQ ID NO: 1384 - DNA, 23 bp, Homo sapiens
cctcccaaactaaaacaattctc

SEQ ID NO: 1385 - DNA, 23 bp, Homo sapiens
caaaacaaaaccctatctccaaa

SEQ ID NO: 1386 - DNA, 22 bp, Homo sapiens
accatcccaactccctatcttt

SEQ ID NO: 1387 - DNA, 24 bp, Homo sapiens
acaaaactctacaacaacacctcc

SEQ ID NO: 1388 - DNA, 26 bp, Homo sapiens
aaaacaaaaccacaactataaaaaca

SEQ ID NO: 1389 - DNA, 23 bp, Homo sapiens
taaaatcaccacccaaaaaacat

SEQ ID NO: 1390 - DNA, 26 bp, Homo sapiens
tttaataaaattttccctcccttacc

SEQ ID NO: 1391 - DNA, 23 bp, Homo sapiens
cttctactcaaacaccaacacca

SEQ ID NO: 1392 - DNA, 24 bp, Homo sapiens
aaaatcaaatccaaataacatccc

SEQ ID NO: 1393 - DNA, 25 bp, Homo sapiens
ctcccaccactttactaaaaactcc

SEQ ID NO: 1394 - DNA, 21 bp, Homo sapiens
taccacttcccaactcttccc

SEQ ID NO: 1395 - DNA, 23 bp, Homo sapiens
accctacatcccaacactaaaaa

SEQ ID NO: 1396 - DNA, 26 bp, Homo sapiens
aaatacctacaaactcctaaaaccca

SEQ ID NO: 1397 - DNA, 21 bp, Homo sapiens
aacaacacccatatcaaccca

SEQ ID NO: 1398 - DNA, 22 bp, Homo sapiens
aacctctcccaacctcaatttt

SEQ ID NO: 1399 - DNA, 29 bp, Homo sapiens
actaataaccactctcacttctctacttt

SEQ ID NO: 1400 - DNA, 29 bp, Homo sapiens
aaacaaactcactaaaatctaaataaccc

SEQ ID NO: 1401 - DNA, 27 bp, Homo sapiens
tttttaaaaaacctataaaaccaatcc

SEQ ID NO: 1402 - DNA, 23 bp, Homo sapiens
aaccatcctccacctcaacttta

SEQ ID NO: 1403 - DNA, 23 bp, Homo sapiens
cctacaaccctactacccctacc

SEQ ID NO: 1404 - DNA, 23 bp, Homo sapiens
ctaacacaaaaaccacactccaa

SEQ ID NO: 1405 - DNA, 24 bp, Homo sapiens
cccaaaaactaaacttcctctaaa

SEQ ID NO: 1406 - DNA, 20 bp, Homo sapiens
acactaccaccaccaccatc

SEQ ID NO: 1407 - DNA, 26 bp, Homo sapiens
aaacatttcacaattcctatctcaaa

SEQ ID NO: 1408 - DNA, 27 bp, Homo sapiens
atattctctctcattctacaccattcc

SEQ ID NO: 1409 - DNA, 26 bp, Homo sapiens
aatcctatactcaataccatttctcc

SEQ ID NO: 1410 - DNA, 24 bp, Homo sapiens
acaaccacaccaaaccctatatct

SEQ ID NO: 1411 - DNA, 23 bp, Homo sapiens
acttccaacccttaaaacaaaca

SEQ ID NO: 1412 - DNA, 22 bp, Homo sapiens
tcccaaaaaacaacttccctaa

SEQ ID NO: 1413 - DNA, 23 bp, Homo sapiens
ttattttaaacccaatcttccct

SEQ ID NO: 1414 - DNA, 25 bp, Homo sapiens
ttacaatataatcctccccaaaatc

SEQ ID NO: 1415 - DNA, 24 bp, Homo sapiens
caaatttcccaatttaattcctca

SEQ ID NO: 1416 - DNA, 27 bp, Homo sapiens
aattaaacacaacactcaattaccaaa

SEQ ID NO: 1417 - DNA, 24 bp, Homo sapiens
caacacacaaaaaccaactacaaa

SEQ ID NO: 1418 - DNA, 25 bp, Homo sapiens
ataccattacaaaaacaacccaaca

SEQ ID NO: 1419 - DNA, 24 bp, Homo sapiens
acacatatcccctctctaatcctc

SEQ ID NO: 1420 - DNA, 23 bp, Homo sapiens
aaaccccatacccttaaacccta

SEQ ID NO: 1421 - DNA, 23 bp, Homo sapiens
acctcaatcttctcatcaacaaa

SEQ ID NO: 1422 - DNA, 23 bp, Homo sapiens
cccaaccaataaacaacaaaaat

SEQ ID NO: 1423 - DNA, 22 bp, Homo sapiens
cctcacaaactaccctcaaacc

SEQ ID NO: 1424 - DNA, 23 bp, Homo sapiens
taaacaccaacttctctcccttt

SEQ ID NO: 1425 - DNA, 23 bp, Homo sapiens
ctcctcccctactcattttaaaa

SEQ ID NO: 1426 - DNA, 23 bp, Homo sapiens
tttccaaactaaaccaccacaaa

SEQ ID NO: 1427 - DNA, 27 bp, Homo sapiens
taacacctaattttatataccccaacc

SEQ ID NO: 1428 - DNA, 25 bp, Homo sapiens
actccatctaacttctttctccctt

SEQ ID NO: 1429 - DNA, 23 bp, Homo sapiens
aactttcaatacatccaccacca

SEQ ID NO: 1430 - DNA, 23 bp, Homo sapiens
caaaacccataaaaattcaaaaa

SEQ ID NO: 1431 - DNA, 17 bp, Homo sapiens
cccctactcctccccac

SEQ ID NO: 1432 - DNA, 18 bp, Homo sapiens
ttcctcctccaacccctc

SEQ ID NO: 1433 - DNA, 22 bp, Homo sapiens
ccctaacacaactctacccaat

SEQ ID NO: 1434 - DNA, 21 bp, Homo sapiens
cctccccataccaacttaaaa

SEQ ID NO: 1435 - DNA, 26 bp, Homo sapiens
aatcatctaaaataaaaacacccaac

SEQ ID NO: 1436 - DNA, 23 bp, Homo sapiens
accaaatctataccaaaatcccc

SEQ ID NO: 1437 - DNA, 24 bp, Homo sapiens
aaattaactcctcaaaacacccac

SEQ ID NO: 1438 - DNA, 23 bp, Homo sapiens
aaacccaaaactacctccaaatc

SEQ ID NO: 1439 - DNA, 23 bp, Homo sapiens
aaccaaaccaaactcccaataaa

SEQ ID NO: 1440 - DNA, 23 bp, Homo sapiens
ttcaacctctctccactaaaacc

SEQ ID NO: 1441 - DNA, 23 bp, Homo sapiens
acccatctcccattacataaaac

SEQ ID NO: 1442 - DNA, 23 bp, Homo sapiens
ctaaacacccaaaccaacaactc

SEQ ID NO: 1443 - DNA, 23 bp, Homo sapiens
accacatacaccaaatacccaaa

SEQ ID NO: 1444 - DNA, 23 bp, Homo sapiens
cctcaacaaataaaccccaaaat

SEQ ID NO: 1445 - DNA, 23 bp, Homo sapiens
ccacccctcaactatttattcac

SEQ ID NO: 1446 - DNA, 27 bp, Homo sapiens
ttccaatcacattacataaacaaataa

SEQ ID NO: 1447 - DNA, 28 bp, Homo sapiens
tctaaaaacctaccactaatattcacaa

SEQ ID NO: 1448 - DNA, 25 bp, Homo sapiens
taaaccctaaactaccataaaacca

SEQ ID NO: 1449 - DNA, 28 bp, Homo sapiens
aaaattaactactctcacctaacaaacc

SEQ ID NO: 1450 - DNA, 22 bp, Homo sapiens
tttcataacaacccaaaatcca

SEQ ID NO: 1451 - DNA, 25 bp, Homo sapiens
aaacactaaccataaaccaaacact

SEQ ID NO: 1452 - DNA, 28 bp, Homo sapiens
caacactatacctaaacaaccttaactt

SEQ ID NO: 1453 - DNA, 23 bp, Homo sapiens
aaacttcaactaacaaaccccac

SEQ ID NO: 1454 - DNA, 23 bp, Homo sapiens
cttatcccaaaaaacaaaaccaa

SEQ ID NO: 1455 - DNA, 24 bp, Homo sapiens
ttccaaccctcacctaactttatc

SEQ ID NO: 1456 - DNA, 25 bp, Homo sapiens
cctattcaaatcaactccttctaaa

SEQ ID NO: 1457 - DNA, 23 bp, Homo sapiens
cacaaacaaacactccaaaaatc

SEQ ID NO: 1458 - DNA, 24 bp, Homo sapiens
cccaaaatctccataaactaaaaa

SEQ ID NO: 1459 - DNA, 23 bp, Homo sapiens
aaaatccctctcacccattaaca

SEQ ID NO: 1460 - DNA, 23 bp, Homo sapiens
aaaaaccacaaccctaaaaaacc

SEQ ID NO: 1461 - DNA, 23 bp, Homo sapiens
aacctcttcaaacaacccaaata

SEQ ID NO: 1462 - DNA, 23 bp, Homo sapiens
ccctatcctaatcacccaactaa

SEQ ID NO: 1463 - DNA, 23 bp, Homo sapiens
ccaccaaacatacaattccaact

SEQ ID NO: 1464 - DNA, 24 bp, Homo sapiens
attctatccctatcctaatcaccc

SEQ ID NO: 1465 - DNA, 24 bp, Homo sapiens
actctctaaaaccaccatcacacc

SEQ ID NO: 1466 - DNA, 23 bp, Homo sapiens
ataaacctccccctcaactaaaa

SEQ ID NO: 1467 - DNA, 25 bp, Homo sapiens
cactacctaacactctaactcccaa

SEQ ID NO: 1468 - DNA, 26 bp, Homo sapiens
tccaattccctaatatacctatttca

SEQ ID NO: 1469 - DNA, 23 bp, Homo sapiens
accaacacaatctctacacccac

SEQ ID NO: 1470 - DNA, 24 bp, Homo sapiens
aacctcaaaccttaacaattcaca

SEQ ID NO: 1471 - DNA, 22 bp, Homo sapiens
aattccctacactcatcccctt

SEQ ID NO: 1472 - DNA, 20 bp, Homo sapiens
ccccactaaacccttaaccc

SEQ ID NO: 1473 - DNA, 19 bp, Homo sapiens
aaaactctcccctctcccc

SEQ ID NO: 1474 - DNA, 25 bp, Homo sapiens
aaaatccaaactccttacctctaat

SEQ ID NO: 1475 - DNA, 27 bp, Homo sapiens
taaacattctatacctacttcccaaaa

SEQ ID NO: 1476 - DNA, 28 bp, Homo sapiens
taaacaataccaactaattccacttttc

SEQ ID NO: 1477 - DNA, 23 bp, Homo sapiens
attccttcctaacactccctcat

SEQ ID NO: 1478 - DNA, 25 bp, Homo sapiens
aactcactcctcacaaaatactcac

SEQ ID NO: 1479 - DNA, 27 bp, Homo sapiens
aattctaaaaatccaaaattctaaacc

SEQ ID NO: 1480 - DNA, 23 bp, Homo sapiens
atttctaaaaccccaaaacctcc

SEQ ID NO: 1481 - DNA, 26 bp, Homo sapiens
tctatacccactaatacaaacccaaa

SEQ ID NO: 1482 - DNA, 25 bp, Homo sapiens
aacacacacacaaaaaacctataca

SEQ ID NO: 1483 - DNA, 24 bp, Homo sapiens
acatacacacacactcaaacacac

SEQ ID NO: 1484 - DNA, 21 bp, Homo sapiens
cccccttaaaccctaaactca

SEQ ID NO: 1485 - DNA, 23 bp, Homo sapiens
cccacacaaaaccaaaaaataaa

SEQ ID NO: 1486 - DNA, 23 bp, Homo sapiens
tcacatcaacctccccaaatact

SEQ ID NO: 1487 - DNA, 22 bp, Homo sapiens
aataccaccttccacaaaccac

SEQ ID NO: 1488 - DNA, 20 bp, Homo sapiens
aacccaatctcacaacaccc

SEQ ID NO: 1489 - DNA, 25 bp, Homo sapiens
aaaacctactcacaaccttaactca

SEQ ID NO: 1490 - DNA, 23 bp, Homo sapiens
aacccaaccaataaaccaaaacc

SEQ ID NO: 1491 - DNA, 24 bp, Homo sapiens
ctaactccctaccattatccacca

SEQ ID NO: 1492 - DNA, 24 bp, Homo sapiens
actccttacccactccaaactaaa

SEQ ID NO: 1493 - DNA, 23 bp, Homo sapiens
aaccaacccaaattcaaaactaa

SEQ ID NO: 1494 - DNA, 25 bp, Homo sapiens
aattcctaactccctaatccctacc

SEQ ID NO: 1495 - DNA, 22 bp, Homo sapiens
cctaactcaaccataaacccca

SEQ ID NO: 1496 - DNA, 25 bp, Homo sapiens
aaattctatactcccctcaaaaatc

SEQ ID NO: 1497 - DNA, 25 bp, Homo sapiens
aaccttccaaatactaccctaactc

SEQ ID NO: 1498 - DNA, 23 bp, Homo sapiens
caaaacaaaaccctatctctcaa

SEQ ID NO: 1499 - DNA, 26 bp, Homo sapiens
acctaaccaatcttctcttcttacaa

SEQ ID NO: 1500 - DNA, 20 bp, Homo sapiens
tcctccaacctaaacctccc

SEQ ID NO: 1501 - DNA, 41 bp, Homo sapiens
cccctggagcggcccctgggcgaggtgtacctggacagcag

SEQ ID NO: 1502 - RNA, 41 nt, Homo sapiens
  misc_feature (1)...(41): n=t
uuuunggagugguuuungggcgaggngnauunggauaguag

SEQ ID NO: 1503 - RNA, 41 nt, Homo sapiens; identical to SEQ ID NO: 1502
  misc_feature (1)...(41): n=t

SEQ ID NO: 1504 - DNA, 19 bp, Artificial Sequence
  LSO primer
aaaaacctcrccaaaaacc

SEQ ID NO: 1505 - DNA, 21 bp, Artificial Sequence
  ASO1 primer
gctccacataaacctatcatc

SEQ ID NO: 1506 - DNA, 21 bp, Artificial Sequence
  ASO2 primer
actccacataaacctatcatc

(19) United States
(12) Patent Application Publication
(10) Pub. No.: US 2005/0026183 A1
(43) Pub. Date: Feb. 3, 2005

(54) Methods and compositions for diagnosing conditions associated with specific DNA methylation patterns

(76) Inventors: Jian-Bing Fan, San Diego, CA (US); Marina Bibikova, San Diego, CA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 10/845,667

(22) Filed: May 14, 2004

Related U.S. Application Data
(60) Provisional application No. 60/471,488, filed on May 15, 2003.

(51) Int. Cl. C12Q 1/68; G06F 19/00; G01N 33/48; G01N 33/50

(52) U.S. Cl. 435/6; 702/20

(57) ABSTRACT

The present invention provides a method for identification of differentially methylated genomic CpG dinucleotide sequences associated with cancer in an individual by obtaining a biological sample comprising genomic DNA from the individual measuring the level or pattern of methylated genomic CpG dinucleotide sequences for two or more of the genomic targets in the sample, and comparing the level of methylated genomic CpG dinucleotide sequences in the sample to a reference level of methylated genomic CpG dinucleotide sequences, wherein a difference in the level or pattern of methylation of the genomic CpG dinucleotide sequences in the sample compared to the reference level identifies differentially methylated genomic CpG dinucleotide sequences associated with cancer. As disclosed herein, the methods of the invention have numerous diagnostic and prognostic applications. The methods of the invention can be combined with a miniaturized array platform that allows for a high level of assay multiplexing and scalable automation for sample handling and data processing. Also provided by the invention are genomic targets and corresponding nucleic acid probes that are useful in the methods of the invention as they enable detection of differentially methylated genomic CpG dinucleotide sequences associated with cancer, for example, adenocarcenomas and sqamous cell carcinomas of the lung.

This application is based on, and claims the benefit of, U.S. Provisional Application No. 60/471,488, filed May 15, 2003, entitled METHODS AND COMPOSITIONS FOR DIAGNOSING CANCER, which is incorporated herein by reference.

This invention was made with government support under grant number 1 R43 CA097851 -01 awarded by the National Institutes of Health. The United States Government has certain rights in this invention.

### FIELD OF THE INVENTION

The present invention relates to conditions characterized by differentially methylated genomic CpG dinucleotide sequences and, in particular, to diagnostic and prognostic methods that exploit the presence of such genomic DNA sequences that exhibit altered CpG methylation patterns.

### BACKGROUND OF THE INVENTION

Methylation of DNA is widespread and plays a critical role in the regulation of gene expression in development, differentiation and diseases such as multiple sclerosis, diabetes, schizophrenia, aging, and cancers. Methylation in particular gene regions, for example in their promoters, can inhibit the expression of these genes. Recent work has shown that the gene silencing effect of methylated regions is accomplished through the interaction of methylcytosine binding proteins with other structural components of chromatin which, in turn, makes the DNA inaccessible to transcription factors through histone deacetylation and chromatin structure changes. Differentially methylated CpG islands have long been thought to function as genomic imprinting control regions (ICRs).

Deregulation of imprinting has been implicated in several developmental disorders. Identification of the ICRs in a large number of human genes and their regulation patterns during development can shed light on genomic imprinting as well as other fundamental epigenetic control mechanisms. Moreover, rapid advances in genomics, both in terms of technology, for example, high-throughput low-cost capillary sequencers and microarray technologies, as well as in terms of availability of information, for example, information gained by virtue of whole genome sequencing, bioinformatics tools and databases, have paved the way for new opportunities in epigenetic studies. For example, it is known that random autosomal inactivation is one of the mechanisms that mammals use to achieve gene dosage control, in addition to random X-chromosome inactivation in females and genomic imprinting. However, genes belonging to this category are just now emerging, with only a few identified so far. Technologies are needed that can provide a systematic survey for identification of genes regulated by this kind of random monoallelic expression control, and determination of when and how such genes are regulated through a wide screening of samples from different tissues or different disease stages.

A Human Epigenome Consortium was formed in 1999 with a mission to systematically map and catalogue the genomic positions of distinct methylation variants. It is likely that large-scale discovery of methylation patterns through de novo DNA sequencing of bisulfite-treated DNA is be carried out in the near future. This would provide a resource for methylation studies analogous to SNP databases for genetic studies, and would be expected to greatly increase the demand for high-throughput, cost-effective methods of carrying out site-specific methylation assays. A publicly accessible database, which carries information about methylation patterns in various biologically significant samples would be the first outcome of these efforts. There is a need for methods for analysis of large sample sets useful for discovering such associations.

Presently, the analysis of DNA methylation patterns in genomic DNA has been significantly hampered by the fact that methylation information is not retained during standard DNA amplification steps such as PCR or biological amplification by cloning in bacteria. Therefore, DNA methylation analysis methods generally rely on a methylation-dependent modification of the original genomic DNA before any amplification step. A battery of DNA methylation detection methods has been developed, including methylation-specific enzyme digestion (Singer-Sam, et al., Nucleic Acids Res. 18(3): 687 (1990), Taylor, et al., Leukemia 15(4): 583-9 (2001)), bisulfite DNA sequencing (Frommer, et al., Proc Natl Acad Sci U S A. 89(5): 1827-31 (1992), Feil, et al., Nucleic Acids Res. 22(4): 695-6 (1994)), methylation-specific PCR (MSP) (Herman, et al., Proc Natl Acad Sci U S A. 93(18): 9821-6 (1996)), methylation-sensitive single nucleotide primer extension (MS-SnuPE) (Gonzalgo, et al., Nucleic Acids Res. 25(12): 2529-31 (1997)), restriction landmark genomic scanning (RLGS) (Kawai, Mol Cell Biol. 14(11): 7421-7 (1994), Akama, et al., Cancer Res. 57(15): 3294-9 (1997)), and differential methylation hybridization (DMH) (Huang, et al., Hum Mol Genet. 8(3): 459-70 (1999)). However, there exists a need for methods that can combine random access to sequences in the genome with very high throughput, which is beneficial for analyzing methylation patterns at high resolution in large sample sets. This invention satisfies this need and provides related advantages as well.

### SUMMARY OF THE INVENTION

The present invention provides a method for identification of differentially methylated genomic CpG dinucleotide sequences associated with cancer in an individual by obtaining a biological sample comprising genomic DNA from the individual measuring the level or pattern of methylated genomic CpG dinucleotide sequences for two or more of the genomic targets in the sample, and comparing the level of methylated genomic CpG dinucleotide sequences in the sample to a reference level of methylated genomic CpG dinucleotide sequences, wherein a difference in the level or pattern of methylation of the genomic CpG dinucleotide sequences in the sample compared to the reference level identifies differentially methylated genomic CpG dinucleotide sequences associated with cancer. As disclosed herein, the methods of the invention have numerous diagnostic and prognostic applications. The methods of the invention can be combined with a miniaturized array platform that allows for a high level of assay multiplexing and scalable automation for sample handling and data processing. Also provided by the invention are genomic targets and corresponding nucleic acid probes that are useful in the methods of the invention as they enable detection of differentially methylated genomic CpG dinucleotide sequences associated with cancer, for example, adenocarcenomas and sqamous cell carcinomas of the lung.

### BRIEF DESCRIPTION OF THE DRAWINGS

FIG. 1 shows an assembly of a randomly ordered fiber optic array. Panel A shows a collection of bead types, each with a distinct oligonucleotide capture probe, is pooled. An etched fiber optic bundle is dipped into the bead pool, allowing individual beads to assemble into the microwells at the bundle's end. Panel B shows a scanning electron micrograph of an assembled array containing 3 micron diameter silica beads with 5 micron core-to-core spacing between features. The beads are stably associated with the wells under standard hybridization conditions.

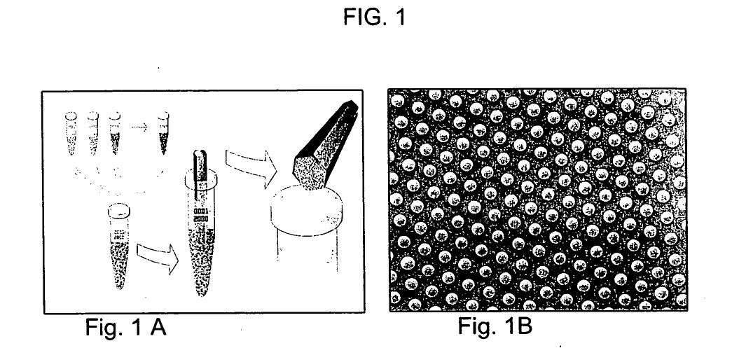

FIG. 2 shows a photograph of a 96-array matrix. Each array is located on the end of an optical fiber bundle containing ˜50,000 individual fibers. The spacing of the arrays matches that of a 96-well plate, allowing96 separate samples to be processed simultaneously.

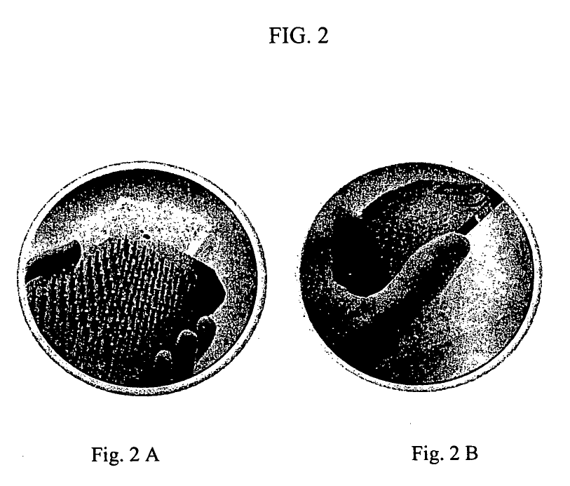

FIG. 3 shows Illumina's SNP genotyping format.

FIG. 4 shows an oligonucleotide design scheme.

FIG. 5 shows plasmid controls used in the assay development. Unmethylated (green), semi-methylated (yellow) and fully-methylated (red) plasmid loci can be correctly scored in the human genomic DNA background.

FIG. 6 shows bisulfite conversion of DNA monitored with internal controls. Top panel: unconverted DNA; bottom panel: DNA after bisulfite conversion. Query oligonucleotides for converted plasmid loci (yellow) and unconverted genomic loci (green) are present in both assays. After bisulfite conversion, signal corresponding to unconverted loci disappears, and signal from converted loci becomes detectable.

FIG. 7 shows methylation assay development and data processing. Left panel: unmethylated, semi-methylated, and fully-methylated loci on the plasmids can be distinguished in the human genomic DNA background. These plasmid DNAs were spiked into human genomic DNA at a 1:1 molar ratio. Right panel: each data point is represented in a red/green/yellow plot, where red indicates a methylated state, green—unmethylated, and yellow—semi-methylated. The whole left panel (bar graph) is represented by one column on a red/green/yellow plot.

FIG. 8 shows reproducible methylation detection in two human reference DNAs: unmethylated (left panel) and methylated (right panel). The red color indicates a methylated state, green—unmethylated, and yellow—semi-methylated. White squares represent the loci with low intensity values, for which the methylation status call could not be made. In the case of amplified gDNA (left panel), some genomic loci may become underrepresented after amplification procedure.

FIG. 9 shows methylation measurement in 15 Coriell genomic DNAs and the reference DNAs.

FIG. 10 shows methylation status of any particular locus determined using a clustering algorithm. Panel A shows raw intensity data (of each bead) for one locus across all 96 replicates. Panel B shows analyzed clusters. Unrethylated, methylated and semi-methylated loci can be distinguished and called correctly by this algorithm.

FIG. 11 shows a schematic overview of a methylation assay that incorporates bisulfite conversion and a bead array format.

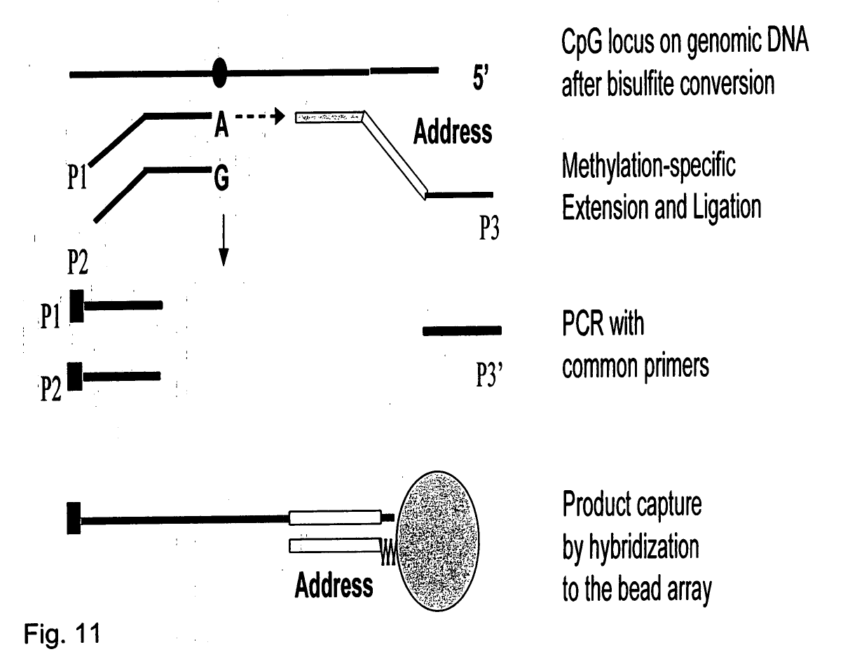

FIG. 12 shows reference samples for a methylation assay encompassing amplified genomic DNA in Panel A and corresponding in vitro methylated genomic DNA in Panel B.

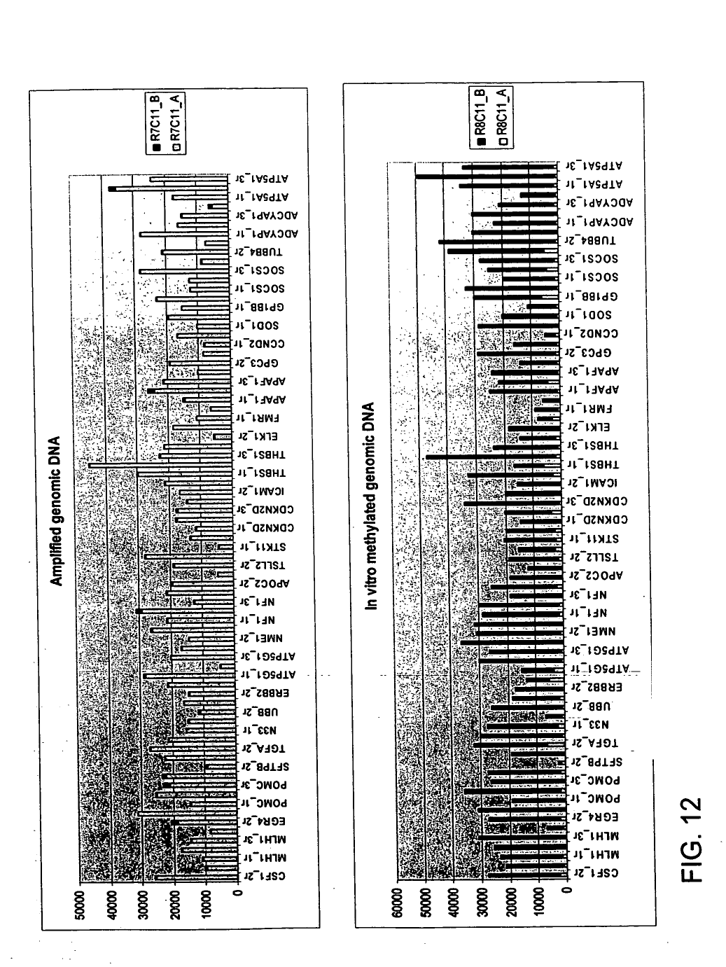

FIG. 13 shows correlation in methylation status between replicates of lung cancer clinical samples containing 389 loci across four independent arrays.

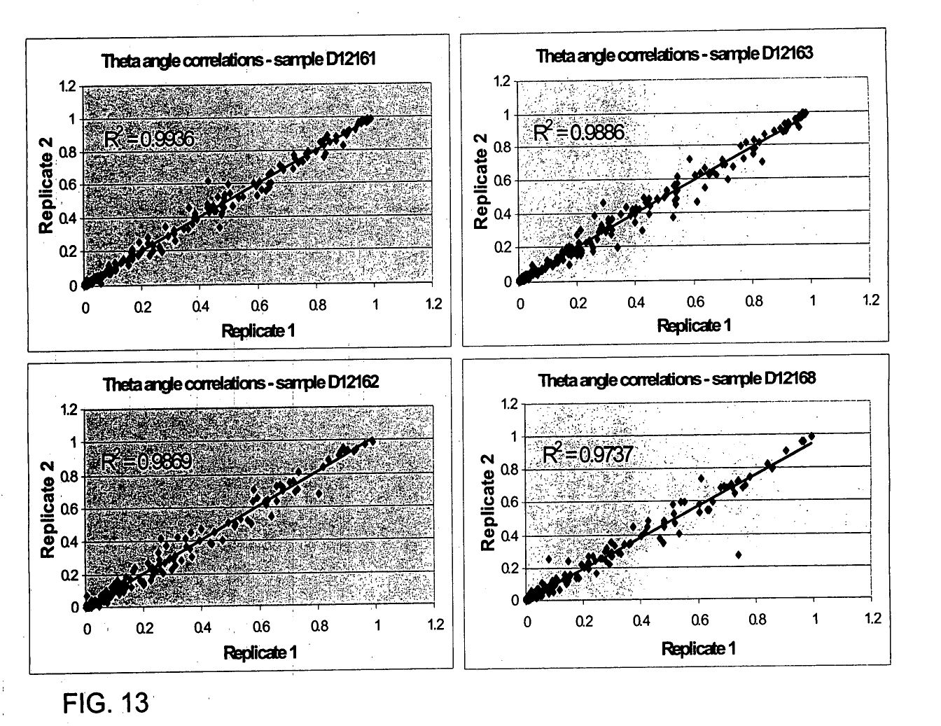

FIG. 14 shows reproducibility of the methylation assay between technical replicates as observed in 46 lung cancer clinical samples containing 389 loci across four independent arrays.

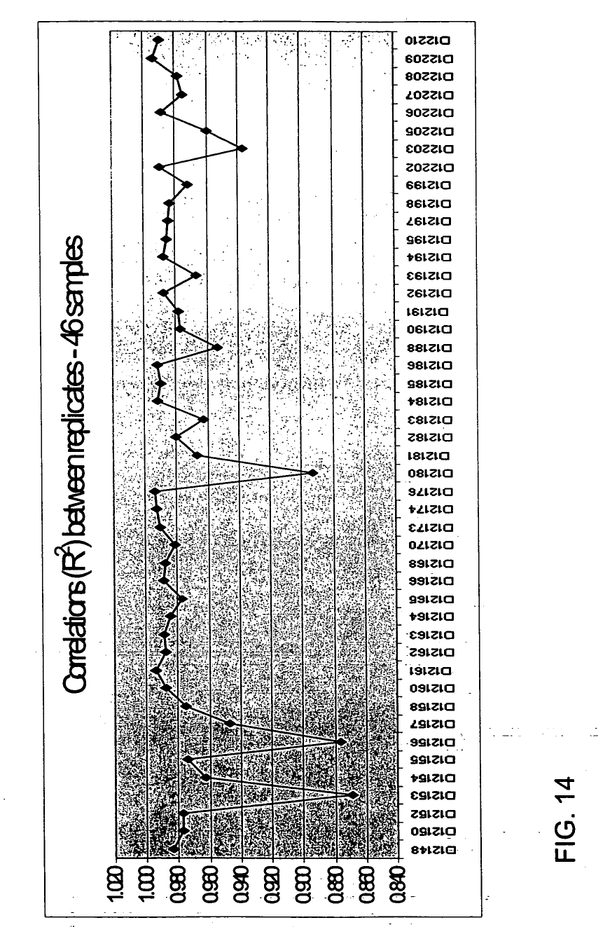

FIG. 15 shows methylation status of two housekeeping genes located on the X chromosome. In females X inactivation correlates with promoter methylation, and methylation pattern determined for both genes in 46 samples (in duplicates) allows to match the methylation status of the promoter with the gender of the sample source.

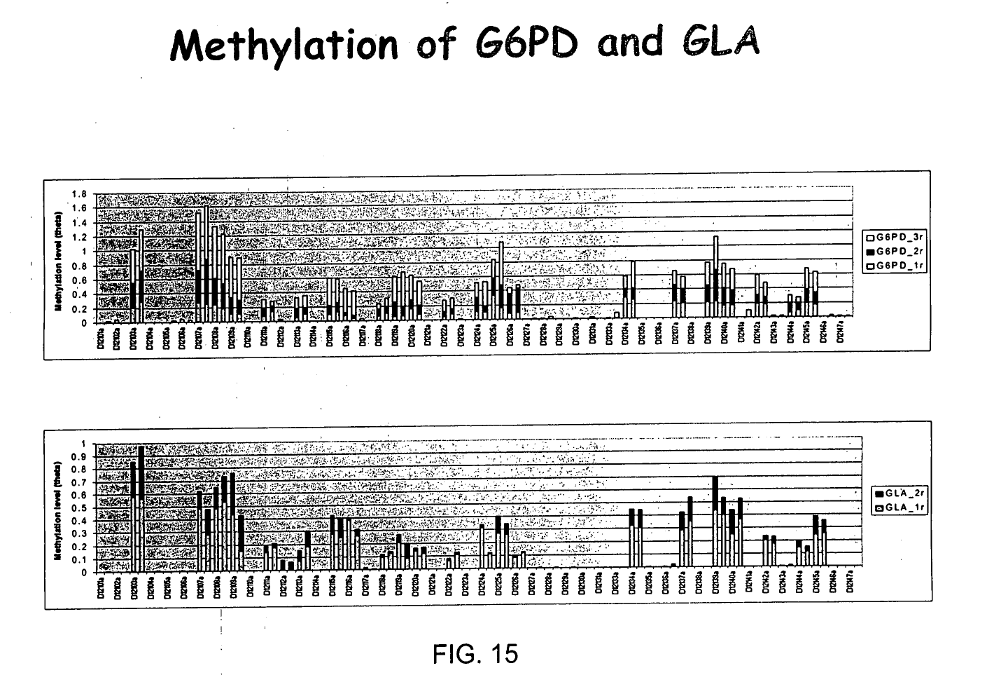

FIG. 16 shows correlation between methylation levels and gender based on methylation status of 6 genes located on the X-chromosome as monitored in 46 samples.

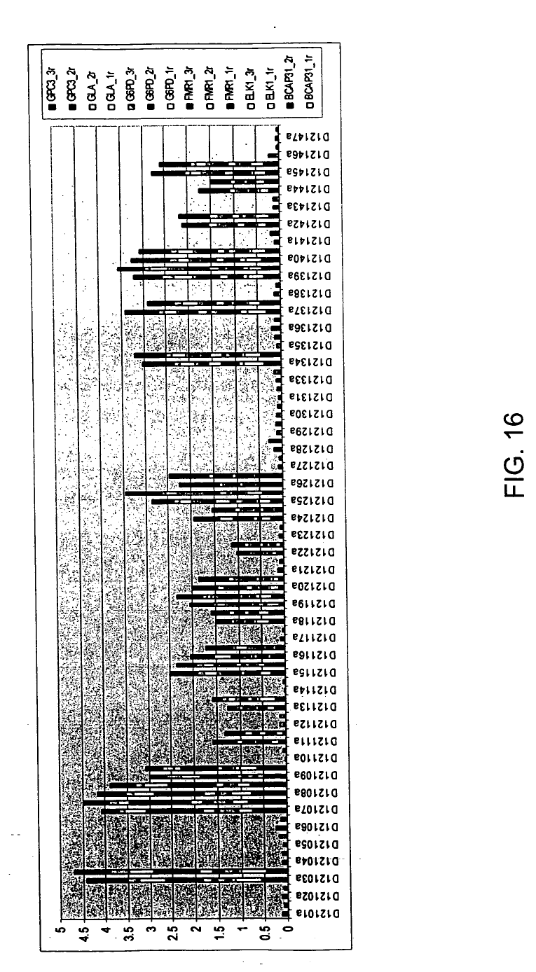

FIG. 17 shows methylation profiling in 46 lung cancer and matched normal tissues based on interrogation of 162 CpG sites. Unmethylated (green), semi-methylated (yellow), methylated (red).

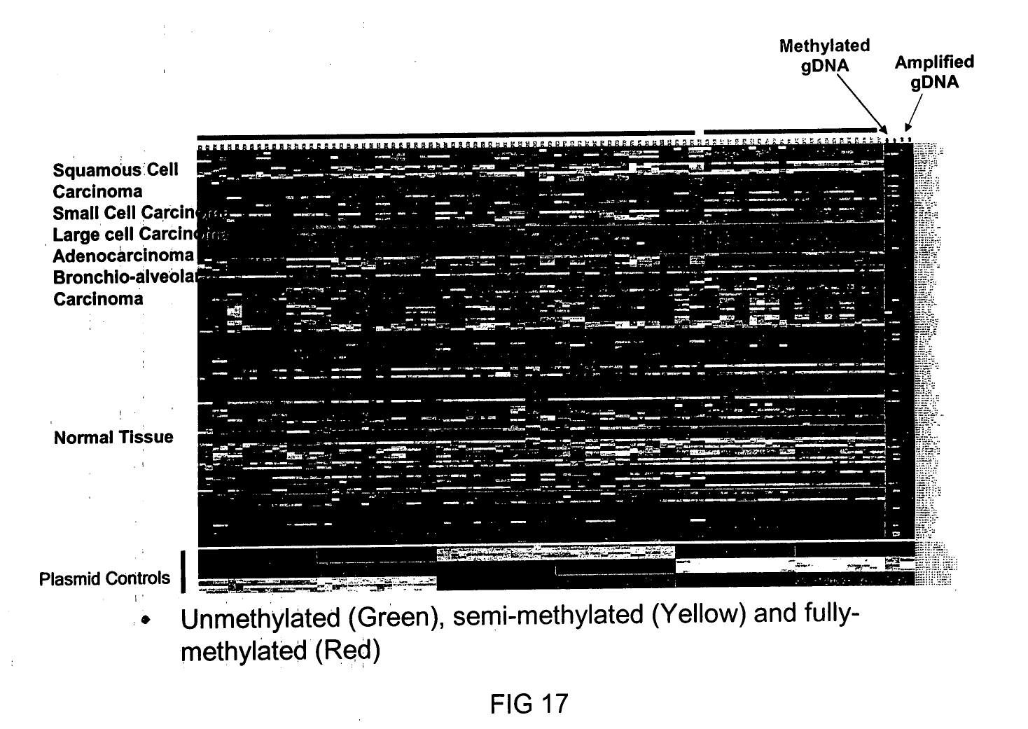

FIG. 18 shows distinct methylation patterns observed for 14 markers in sqamous cell carcinoma versus normal matching tissue.

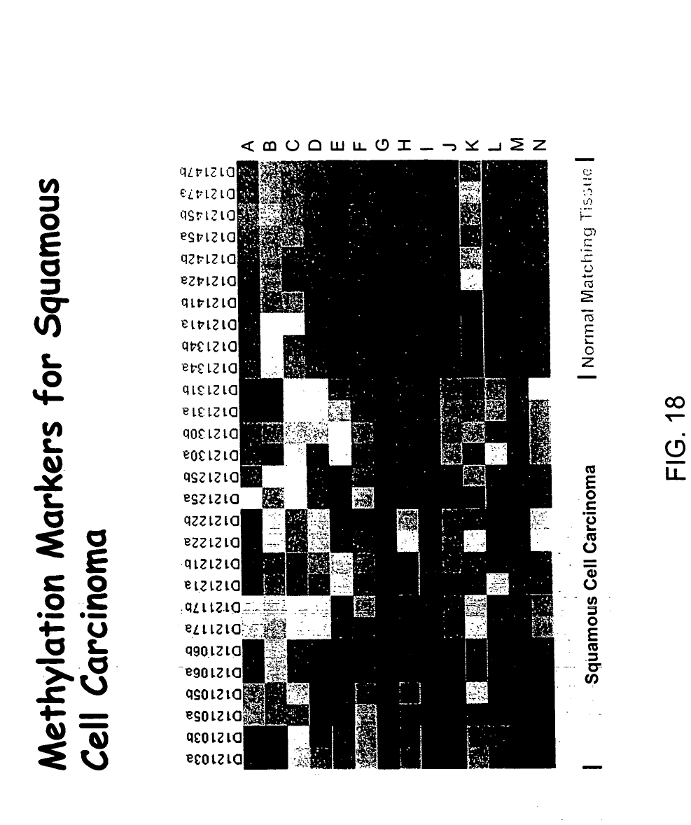

FIG. 19 shows cluster analysis of methylation profiles in 46 lung cancer samples which demonstrates good separation of cancer samples from normal matching pairs.

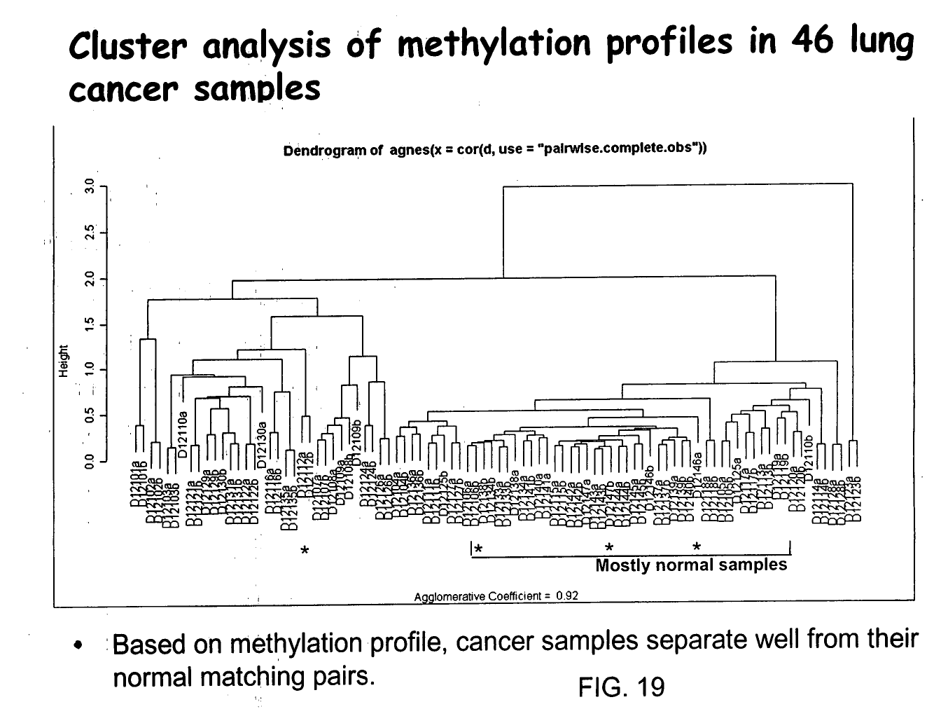

### DETAILED DESCRIPTION OF THE INVENTION

The invention disclosed herein provides diagnostic and prognostic methods for a condition that is characterized by differential methylation of genomic CpG dinucleotide sequences. Also provided are populations of genomic targets and corresponding nucleic acid probes that useful for the detection of differentially methylated genomic CpG dinucleotide sequences that can be correlated to the presence of or susceptibility to cancer in an individual.

The methods of the invention are directed to methods for diagnosing an individual with a condition that is characterized by a level and/or pattern of methylated genomic CpG dinucleotide sequences distinct from the level and/or pattern of methylated genomic CpG dinucleotide sequences exhibited in the absence of the particular condition. This invention also is directed to methods for predicting the susceptibility of an individual to a condition that is characterized by a level and/or pattern of methylated genomic CpG dinucleotide sequences that is distinct from the level and/or pattern of methylated genomic CpG dinucleotide sequences exhibited in the absence of the condition.

In various distinct embodiments, the present invention is based, in part, on the identification of reliable CpG methylation markers for the improved prediction of susceptibility, diagnosis and staging of cancer. The invention provides a population of reliable genomic targets for use in the diagnostic and prognostic methods provided by the present invention. The genomic targets provided by the invention represent gene targets for methylation of genomic CpG dinucleotide sequences associated with cancer. Also provided are nucleic acid probes that correspond to the genomic target sites of the invention and that can be used to detect differential methylation of selected genomic CpG dinucleotide sequences that serve as markers associated with cancer.

The genomic targets and nucleic acid probes provided by the present invention are set forth in Table 1, below, and provide diagnostic and prognostic tools based on their ability to detect differential methylation of selected genomic CpG dinucleotide sequences associated with cancer. In the methods provided by the invention, the genomic targets and nucleic acid probes capable of detecting markers located within the genomic targets can be employed to detect altered levels of methylation of genomic CpG dinucleotide sequences in a biological sample compared to a reference level. Furthermore, the methods of the invention allow for use of the genomic markersand nucleic acid probes for the determination of methylation patterns, which are represented by differential methylation of selected genomic CpG dinucleotide sequences that serve as markers in particular sets or subsets of genomic targets. In embodiments directed to the detection of methylation patterns, it is possible to diagnose or predict the susceptibility of an individual to a specific tumor-type based on the correlation between the pattern and the tumor type.

DNA methylation is a mechanism for changing the base sequence of DNA without altering its coding function. DNA methylation is a heritable, reversible and epigenetic change. Yet, DNA methylation has the potential to alter gene expression, which has profound developmental and genetic consequences. The methylation reaction involves flipping a target cytosine out of an intact double helix to allow the transfer of a methyl group from S adenosylmethionine in a cleft of the enzyme DNA (cystosine-5)-methyltransferase (Klimasauskas et al., Cell 76:357-369, 1994) to form 5-methylcytosine (5-mCyt). This enzymatic conversion is the most common epigenetic modification of DNA known to exist in vertebrates and is essential for normal embryonic development (Bird, Cell 70:5-8, 1992; Laird and Jaenisch, Human Mol. Genet. 3:1487-1495, 1994; and Bestor and Jaenisch, Cell 69:915-926, 1992). The presence of 5-mCyt at CpG dinucleotides has resulted in a 5-fold depletion of this sequence in the genome during vertebrate evolution, presumably due to spontaneous deamination of 5-mCyt to T (Schoreret et al., Proc. Natl. Acad. Sci. USA 89:957-961, 1992). Those areas of the genome that do not show such suppression are referred to as “CpG islands” (Bird, Nature 321:209-213, 1986; and Gardiner-Garden et al., J Mol. Biol. 196:261-282, 1987). These CpG island regions comprise about 1% of vertebrate genomes and also account for about 15% of the total number of CpG dinucleotides. CpG islands are typically between 0.2 to about 1 kb in length and are located upstream of many housekeeping and tissue-specific genes, but may also extend into gene coding regions. Therefore, the methylation of cytosine residues within CpG islands in somatic tissues can modulate gene expression throughout the genome (Cedar, Cell 53:3-4, 1988; Nature 421:686-688, 2003).

Methylation of cytosine residues contained within CpG islands of certain genes has been inversely correlated with gene activity. Thus, methylation of cytosine residues within CpG islands in somatic tissue is generally associated with decreased gene expression and can be the effect a variety of mechanisms including, for example, disruption of local chromatin structure, inhibition of transcription factor-DNA binding, or by recruitment of proteins which interact specifically with methylated sequences indirectly preventing transcription factor binding. Despite a generally inverse correlation between methylation of CpG islands and gene expression, however, most CpG islands on autosomal genes remain unmethylated in the germline and methylation of these islands is usually independent of gene expression. Tissue-specific genes are usually unmethylated at the receptive target organs but are methylated in the germline and in non-expressing adult tissues. CpG islands of constitutively-expressed housekeeping genes are normally unmethylated in the germline and in somatic tissues.

Abnormal methylation of CpG islands associated with tumor suppressor genes can cause decreased gene expression. Increased methylation of such regions can lead to progressive reduction of normal gene expression resulting in the selection of a population of cells having a selective growth advantage. Conversely, decreased methylation (hypomethylation) of oncogenes can lead to modulation of normal gene expression resulting in the selection of a population of cells having a selective growth advantage.

The present invention harnesses the potential of genomic methylation of CpG islands as indicators of the presence of a condition in an individual and provides a reliable diagnostic and/or prognostic method applicable to any condition associated with altered levels or patterns of genomic methylation of CpG islands. CpG islands are contiguous regions of genomic DNA that have an elevated frequency of CpG dinucleotides compared to the rest of the genome. CpG islands are typically, but not always, between about 0.2 to about 1 kb in length, and may be as large as about 3 Kb in length. Generally, for the methods provided by the invention at least two or more, at least three or more, at least four or more CpG dinucleotide sequences are selected that are located within a genomic marker so as to allow for determination of co-methylation status in the genomic DNA of a given tissue sample. Preferably the primary and secondary CpG dinucleotide sequences are co-methylated as part of a larger co-methylated pattern of differentially methylated CpG dinucleotide sequences in the genomic marker. The size of such context regions varies, but generally reflects the size of CpG islands as described above, or the size of a gene promoter region, including the first one or two exons.

With particular regard to cancer, changes in DNA methylation have been recognized as one of the most common molecular alternations in human neoplasia. Hypermethylation of CpG islands located in the promoter regions of tumor suppressor genes is a well-established and common mechanism for gene inactivation in cancer (Esteller, Oncogene 21(35): 5427-40 (2002)). In contrast, a global hypomethylation of genomic DNA is observed in tumor cells; and a correlation between hypomethylation and increased gene expression has been reported for many oncogenes (Feinberg, Nature 301(5895): 89-92 (1983), Hanada, et al., Blood 82(6): 1820-8 (1993)). Thus, a detailed study of methylation pattern in selected, staged tumor samples compared to matched normal tissues from the same patient offers a novel approach to identify unique molecular markers for cancer classification. Monitoring global changes in methylation pattern has been applied to molecular classification in breast cancer (Huang, et al., Hum Mol Genet. 8(3): 459-70 (1999)). In addition, many studies have identified a few specific methylation patterns in tumor suppressor genes, for example, p16, a cyclin-dependent kinase inhibitor, in certain human cancer types (Otterson, et al., Oncogene 11 (6): 1211-6 (1995), Herman, et al., Cancer Res. 55(20): 4525-30 (1995)). Some of the most recent examples include the discoveries of causal relationship between the loss of RUNX3 expression, due to hypermethylation, and gastric cancer (Li, et al., Cell 109(1): 113-24 (2002)); loss of IGF2 imprinting in colorectal cancer (Cui, et al., Science 299(5613): 1753-5 (2003); and reduced Hic gene expression in several types of human cancer (Chen, et al., Nat Genet. 33(2): 197-202 2003), Fujii, et al., Oncogene 16(16): 2159-64 (1998), Kanai, et al., Hepatology 29(3): 703-9 (1999)).

In one embodiment, the invention provides a method for identification of differentially methylated genomic CpG dinucleotide sequences associated with cancer in an individual by obtaining a biological sample comprising genomic DNA from the individual; measuring the level of methylated genomic CpG dinucleotide sequences for two or more of the markers set forth herein and designated as SEQ ID NOS: 1-376 in the sample, and comparing the level of methylated genomic CpG dinucleotide sequences in the sample to a reference level of methylated genomic CpG dinucleotide sequences, wherein a difference in the level of methylation of said genomic CpG dinucleotide sequences in the sample compared to the reference level identifies differentially methylated genomic CpG dinucleotide sequences associated with cancer. In additional embodiments, the level of methylated genomic CpG dinucleotide sequences is measured for one or more, three or more, four or more, five or more, six ore more, seven or more, eight or more, nine or more, ten or more, eleven or more, twelwe or more, thirteen or more, fourteen or more, fifteen or more, twenty or more, twenty-five or more, thirty or more, fifty or more, of the markers set forth herein and designated as SEQ ID NOS: 1-376 in the sample. A subset of the genomic markers or nucleic acid probes of the invention can be one ore more nucleic acid sequences.

The level of methylation of the differentially methylated genomic CpG dinucleotide sequences can provide a variety of information about the cancer and can be used, for example, to diagnose cancer in the individual; to predict the course of the cancer in the individual; to predict the susceptibility to cancer in the individual, to stage the progression of the cancer in the individual; to predict the likelihood of overall survival for the individual; to predict the likelihood of recurrence of cancer for the individual; to determine the effectiveness of a treatment course undergone by the individual.

As described herein, the level of methylation that is detected in a biological sample can be decreased or increased in comparison to the reference level and alterations that increase or decrease methylation can be detected and provide useful prognostic or diagnostic information. For example, hypermethylation of CpG islands located in the promoter regions of tumor suppressor genes have been established as common mechanisms for gene inactivation in cancers (Esteller, Oncogene 21(35): 5427-40 (2002)). Thus, a detailed study of methylation pattern in selected, staged tumor samples compared to matched normal tissues from the same patient can identify unique molecular markers for cancer classification. Furthermore, once identified, such molecular markers.

In addition to detecting levels of methylation, the present invention also allows for the detection of patterns of methylation. It has been confirmed previously that neoplastic cells can exhibit unusual patterns of gene methylation (Feinberg and Vogelstein, Nature 301:89-92 (1983)). Previous genetic studies of various conditions, for example, schizophrenia and bipolar disorder, seemed to implicate regions of particular chromosomes 22, but studies failed to identify a susceptibility gene. Analysis of methylation patterns across these chromosome in biological samples from afflicted individuals can reveal epigenetic changes in the form of altered levels of methylation of subsets of genomic CpG dinucleotide sequences that make up a pattern of affected genomic targets that can be correlated with a condition.

In one embodiment of the invention, an altered level of methylation of genomic CpG dinucleotide sequences is observed only in a subset of the genomic targets set forth in Table 1 and designated SEQ ID NOS: 1-376. In this embodiment, the subset can represent a methylation pattern characteristic of a particular type of cancer. Therefore, as described herein with reference to cancer, methylation patterns can correlated with a particular type, class or origin of a condition and detection and comparison of methylation patterns across samples that share a phenotypic characteristic can be useful to identify further methylation patterns.

In a further embodiment the present invention provides a population of genomic targets comprising nucleic acid sequences designated SEQ ID NOS: 1-376, and set forth in Table 1. Also provided in a distinct, but related embodiment is a population of genomic targets selected from the group consisting of nucleic acid sequences designated SEQ ID NOS: 1-376. The genomic targets are capable of exhibiting altered levels of methylation of genomic CpG dinucleotide sequences that are predictive of the presence or susceptibility of an individual for cancer.

In a further embodiment the invention provides a population of genomic targets comprising a subset of the nucleic acid sequences designated SEQ ID NOS: 1-376, and set forth in Table 1. Differential methylation of genomic CpG dinucleotide sequences in a subset of SEQ ID NOS: 1-376 can be characteristic of a particular type, class or origin of cancer. Detection of differential methylation in a subset of genomic targets can be useful to diagnose or predict susceptibility for a particular type, class or origin of cancer.

Also provided by the present invention is a population of nucleic acid probes capable of detecting methylation of genomic CpG dinucleotide sequences of two or more genomic targets selected from the group consisting of the nucleic acid sequences designated SEQ ID NOS: 1-376, and set forth in Table 1. The population of nucleic acid probes provided by the invention consists of two or more nucleic acid sequences selected from the group consisting of SEQ ID NOS: 377-1880, and set forth in Table 1. The nucleic acid probes of the invention are capable of detecting altered levels of methylation of genomic CpG dinucleotide sequences of two or more genomic targets, wherein altered levels are predictive of the presence or susceptibility of an individual for cancer. Based on the observation that adjacent CpG sites tend to be co-methylated or co-de-methylated, a design scheme can be applied in which a “CG” sequence was used for all the CpG sites within the vicinity of the design, in particular for the landing sites for both ASO and LSO, to target any methylated CpG site; while a “TG” sequence was used for all the CpG sites within the vicinity of the design, to target any un-methylated CpG site. This approach requires two separate LSO oligos, but adds better discrimination between the methylated and unmethylated alleles.

In a further embodiment aimed at determination of patterns of DNA methylation, a population of nucleic acid probes is utilized that is capable of detecting altered levels of methylation of genomic CpG dinucleotide sequences of a subset of a population of two or moregenomic targets. Thus, the detection of differential methylation of genomic CpG dinucleotide sequences in only a subset of genomic targets can be used to identify a pattern that correlates with a particular type, class or origin of cancer.

As disclosed herein, the present invention provides a subset of genomic targets consisting of nucleic acid sequences set forth in Table 2 and designated SEQ ID NOS: []] which exhibits differential methylation of genomic CpG dinucleotide sequences associated with lung squamous cell carcinoma. In a further embodiment, the present invention provides a subset of genomic targets consisting of nucleic acid sequences set forth in Table 3 and designated SEQ ID NOS: [ ] which exhibits differential methylation of genomic CpG dinucleotide sequences associated with lung adenocarcinoma. Also provided a target nucleic acid probes for detection of a subset of genomic targets consisting of nucleic acid sequences designated SEQ ID NOS: [ ] which exhibits differential methylation of genomic CpG dinucleotide sequences associated with squamous cell carcinoma (Table 2). In the presence of differentially methylated genomic CpG dinucleotide sequences, the subsets of genomic targets signify a pattern distinctive of a particular type of cancer, for example, adenocarcinoma or sqamous cell carcinoma of the lung tissue. The p value can be calculated for each individual marker. More than one CpG dinucleotide sequence that serves as genomic marker can be selected from the same gene if desired. Thus a gene can provide the context for more than one genomic CpG dinucleotide sequence such that methylation is determined for more than one CpG dinucleotide sequence within a single gene. The p-value was calculated based on a t-test of the level of methylation [i.e. methylation allele intensity/(methylation allele intensity+un-methylation allele intensity)] among 9 lung squamous cell carcinoma and 5 matching normal samples or 14 other normal samples (Table 2), and 16 lung adenocarcinoma and 11 normal samples (Table 3).

Different panels of markers were generated from different subsets of sample. Majority of the markers are detected with significant p-values in all these subsets of sample, suggesting they are indeed robust markers.

The invention also provides subsets of target nucleic acid probes capable of detecting a pattern of methylation of genomic CpG dinucleotide sequences that is associated with a particular type of cancer. Thus, the invention provides a subset of genomic targets consisting of nucleic acid sequences designated SEQ ID NOS: [ ] which exhibits differential methylation of genomic CpG dinucleotide sequences associated with adenocarcinoma (Table 3). The population of nucleic acid probes capable of detecting altered levels of methylation of genomic CpG dinucleotide sequences of a subset of said two or more genomic targets associated with adenocarcinoma are set forth as SEQ ID NOS: [ ].

In a further embodiment, the invention provides a subset of genomic targets consisting of nucleic acid sequences set forth in Table 2 and designated SEQ ID NOS: [ ] which exhibits differential methylation of genomic CpG dinucleotide sequences associated with sqamous cell carcinoma. The population of nucleic acid probes capable of detecting altered levels of methylation of genomic CpG dinucleotide sequences of a subset of said two or more genomic targets associated with sqamous cell carcinoma are set forth in Table 2 and designated SEQ ID NOS: [ ].

In one embodiment, this invention provides diagnostic markers for cancer. The markers of the invention are genomic sequences having methylation states that are diagnostic or prognostic of the presence or severity of cancer. A list of exemplary genes for which methylation state can be used to determine the presence or severity of cancer is provided in Table 1. Cancer diagnosis or prognosis in a method of the invention can be made in a method of the invention based on the methylation state of particular sequence regions of the gene including, but not limited to, the coding sequence, the 5′-regulatory regions, or other regulatory regions that influence transcription efficiency.

The prognostic methods of the invention are useful for determining if a patient is at risk for recurrence. Cancer recurrence is a concern relating to a variety of types of cancer. For example, of patients undergoing complete surgical removal of colon cancer, 25-40% of patients with stage II colon carcinoma and about 50% of patients with stage III colon carcinoma experience cancer recurrence. One explanation for cancer recurrence is that patients with relatively early stage disease, for example, stage II or stage III, already have small amounts of cancer spread outside the affected organ that were not removed by surgery. These cancer cells, referred to as micrometastases, cannot typically be detected with currently available tests.

The prognostic methods of the invention can be used to identify surgically treated patients likely to experience cancer recurrence so that they can be offered additional therapeutic options, including preoperative or postoperative adjuncts such as chemotherapy, radiation, biological modifiers and other suitable therapies. The methods are especially effective for determining the risk of metastasis in patients who demonstrate no measurable metastasis at the time of examination or surgery.

The prognostic methods of the invention also are useful for determining a proper course of treatment for a patient having cancer. A course of treatment refers to the therapeutic measures taken for a patient after diagnosis or after treatment for cancer. For example, a determination of the likelihood for cancer recurrence, spread, or patient survival, can assist in determining whether a more conservative or more radical approach to therapy should be taken, or whether treatment modalities should be combined. For example, when cancer recurrence is likely, it can be advantageous to precede or follow surgical treatment with chemotherapy, radiation, immunotherapy, biological modifier therapy, gene therapy, vaccines, and the like, or adjust the span of time during which the patient is treated. As described herein, the diagnosis or prognosis of cancer state is typically correlated with the degree to which one or more of the genes in Table I is methylated. Thus, the invention can include a determination made based on the methylation state for the entire set of genes in Table I or a subset of the genes.

Furthermore, as the list in Table I is exemplary, the methylation state of other genes or genomic sequences can also be used in a method of the invention to determine the presence or severity of cancer. Exemplary cancers that can be evaluated using a method of the invention include, but are not limited to hematoporetic neoplasms, Adult T-cell leukemia/lymphoma, Lymphoid Neoplasms, Anaplastic large cell lymphoma, Myeloid Neoplasms, Histiocytoses, Hodgkin Diseases (HD), Precursor B lymphoblastic leukemia/lymphoma (ALL), Acute myclogenous leukemia (AML), Precursor T lymphoblastic leukemia/lymphoma (ALL), Myclodysplastic syndromes, Chronic Mycloproliferative disorders, Chronic lymphocytic leukemia/small lymphocytic lymphoma (SLL), Chronic Myclogenous Leukemia (CML), Lymphoplasmacytic lymphoma, Polycythemia Vera, Mantle cell lymphoma, Essential Thrombocytosis, Follicular lymphoma, Myelofibrosis with Myeloid Metaplasia, Marginal zone lymphoma, Hairy cell leukemia, Hemangioma, Plasmacytoma/plasma cell myeloma, Lymphangioma, Glomangioma, Diffuse large B-cell lymphoma, Kaposi Sarcoma, Hemanioendothelioma, Burkitt lymphoma, Angiosarcoma, T-cell chronic lymphocytic leukemia, Hemangiopericytoma, Large granular lymphocytic leukemia, head & neck cancers, Basal Cell Carcinoma, Mycosis fingoids and sezary syndrome, Squamous Cell Carcinoma, Ceruminoma, Peripheral T-cell lymphoma, Osteoma, Nonchromaffin Paraganglioma, Angioimmunoblastic T-cell lymphoma, Acoustic Neurinoma, Adenoid Cystic Carcinoma, Angiocentric lymphoma, Mucoepidermoid Carcinoma, NK/T-cell lymphoma, Malignant Mixed Tumors, Intestinal T-cell lymphoma, Adenocarcinoma, Malignant Mesothelioma, Fibrosarcoma, Sarcomotoid Type lung cacer, Osteosarcoma, Epithelial Type lung cancer, Chondrosarcoma, Melanoma, cancer of the gastrointestinal tract, olfactory Neuroblastoma, Squamous Cell Carcinoma, Isolated Plasmocytoma, Adenocarcinoma, Inverted Papillomas, Carcinoid, Undifferentiated Carcinoma, Malignant Melanoma, Mucoepidermoid Carcinoma, Adenocarcinoma, Acinic Cell Carcinoma, Gastric Carcinoma, Malignant Mixed Tumor, Gastric Lymphoma, Gastric Stromal Cell Tumors, Amenoblastoma, Lymphoma, Odontoma, Intestinal Stromal Cell tumors, thymus cancers, Malignant Thymoma, Carcinids, Type I (Invasive thymoma), Malignant Mesethelioma, Type II (Thymic carcinoma), Non-mucin producing adenocarcinoma, Squamous cell carcinoma, Lymph epithelioma, cancers of the liver and biliary tract, Squamous Cell Carcinoma, Hepatocellular Carcinoma, Adenocarcinoma, Cholangiocarcinoma, Hepatoblastoma, papillary cancer, Angiosarcoma, solid Bronchioalveolar cancer, Fibrolameller Carcinoma, Small Cell Carcinoma, Carcinoma of the Gallbladder, Intermediate Cell carcinaoma, Large Cell Carcinoma, Squamous Cell Carcinoma, Undifferentiated cancer, cancer of the pancreas, cancer of the female genital tract, Squamous Cell Carcinoma, Cystadenocarcinoma, Basal Cell Carcinoma, Insulinoma, Melanoma, Gastrinoma, Fibrosarcoma, Glucagonamoa, Intaepithelial Carcinoma, Adenocarcinoma Embryonal, cancer of the kidney, Rhabdomysarcoma, Renal Cell Carcinoma, Large Cell Carcinoma, Nephroblastoma (Wilm's tumor), Neuroendocrine or Oat Cell carcinoma, cancer of the lower urinary tract, Adenosquamous Carcinoma, Urothelial Tumors, Undifferentiated Carcinoma, Squamous Cell Carcinoma, Carcinoma of the female genital tract, Mixed Carcinoma, Adenoacanthoma, Sarcoma, Small Cell Carcinoma, Carcinosarcoma, Leiomyosarcoma, Endometrial Stromal Sarcoma, cancer of the male genital tract, Serous Cystadenocarcinoma, Mucinous Cystadenocarcinoma, Sarcinoma, Endometrioid Tumors, Speretocytic Sarcinoma, Embyonal Carcinoma, Celioblastoma, Choriocarcinoma, Teratoma, Clear Cell Carcinoma, Leydig Cell Tumor, Unclassified Carcinoma, Sertoli Cell Tumor, Granulosa-Theca Cell Tumor, Sertoli-Leydig Cell Tumor, Disgerminoma, Undifferentiated Prostatic Carcinoma, Teratoma, Ductal Transitional carcinoma, breast cancer, Phyllodes Tumor, cancer of the bones joints and soft tissue, Paget's Disease, Multiple Myeloma, Insitu Carcinoma, Malignant Lymphoma, Invasive Carcinoma, Chondrosacrcoma, Mesenchymal Chondrosarcoma, cancer of the endocrine system, Osteosarcoma, Adenoma, Ewing Tumor, endocrine Carcinoma, Malignant Giant Cell Tumor, Meningnoma, Adamantinoma, Cramiopharlingioma, Malignant Fibrous Histiocytoma, Papillary Carcinoma, Histiocytoma, Follicular Carcinoma, Desmoplastic Fibroma, Medullary Carcinoma, Fibrosarcoma, Anoplastic Carcinoma, Chordoma, Adenoma, Hemangioendothelioma, Memangispericytoma, Pheochromocytoma, Liposarcoma, Neuroblastoma, Paraganglioma, Histiocytoma, Pineal cancer, Rhabdomysarcoms, Pineoblastoma, Leiomyosarcoma, Pineocytoma, Angiosarcoma, skin cancer, cancer of the nervous system, Melanoma, Schwannoma, Squamous cell carcinoma, Neurofibroma, Basal cell carcinoma, Malignant Periferal Nerve Sheath Tumor, Merkel cell carcinoma, Sheath Tumor, Extramamary Paget's Disease, Astrocytoma, Paget's Disease of the nipple, Fibrillary Astrocytoma, Glioblastoma Multiforme, Brain Stem Glioma, Cutaneous T-cell lymphoma, Pilocytic Astrocytoma, Xanthorstrocytoma, Histiocytosis, Oligodendroglioma, Ependymoma, Gangliocytoma, Cerebral Neuroblastoma, Central Neurocytoma, Dysembryoplastic Neuroepithelial Tumo,r Medulloblastoma, Malignant Meningioma, Primary Brain Lymphoma, Primary Brain Germ Cell Tumor, cancers of the eye, Squamous Cell Carcinoma, Mucoepidermoid Carcinoma, Melanoma, Retinoblastoma, Glioma, Meningioma, cancer of the heart, Myxoma, Fibroma, Lipoma, Papillary Fibroelastoma, Rhasdoyoma, or Angiosarcoma among others.

This invention provides methods for determining a prognosis for survival for a cancer patient. One method involves (a) measuring a level of methylation for one or more of the genes listed in Table 1 in a neoplastic cell-containing sample from the cancer patient, and (b) comparing the level of methylation in the sample to a reference level of methylation for the gene, wherein a low level of methylation for the gene in the sample correlates with increased survival of the patient.

Another method involves (a) measuring a level of methylation for one or more of the genes listed in Table 1 in a neoplastic cell-containing sample from the cancer patient, and (b) classifying the patient as belonging to either a first or second group of patients, wherein the first group of patients having low levels of methylation for a gene is classified as having an increased likelihood of survival compared to the second group of patients having high level of methylation for a gene.

The invention also provides a method for monitoring the effectiveness of a course of treatment for a patient with cancer. The method involves (a) determining a level of one or more of the genes listed in Table 1 in a neoplastic cell containing sample from the cancer patient prior to treatment, and (b) determining the level of methylation for the gene in a neoplastic cell-containing sample from the patient after treatment, whereby comparison of the level of methylation for the gene prior to treatment with the level of methylation for the gene after treatment indicates the effectiveness of the treatment.

As used herein, the term “reference level” refers to a control level of expression of a marker used to evaluate a test level of expression of a biomarker in a neoplastic cell-containing sample of a patient. For example, when the level of methylation of one or more genes, referred to herein as “genomic targets,” in the neoplastic cells of a patient are higher than the reference level of methylation for the genes, the cells are considered to have a low level of expression of the gene. Conversely, when the level of methylation of one or more genes in the neoplastic cells of a patient are lower than the reference level, the cells are considered to have a low level of expression, of the gene.

A reference level can be determined based on reference samples collected from age-matched normal classes of adjacent tissues, and with normal peripheral blood lymphocytes. The reference level can be determined by any of a variety of methods, provided that the resulting reference level accurately provides a level of a marker above which exists a first group of patients having a different probability of survival than that of a second group of patients having levels of the biomarker below the reference level. The reference level can be determined by, for example, measuring the level of expression of a biomarker in non-tumorous cells from the same tissue as the tissue of the neoplastic cells to be tested. The reference level can also be a level of a biomarker of in vitro cultured cells which can be manipulated to simulate tumor cells, or can be manipulated in any other manner which yields expression levels which accurately determine the reference level. The reference level can also be determined by comparison of the level of a biomarker, such as methylation of one or more genes, in populations of patients having the same cancer. This can be accomplished, for example, by histogram analysis, in which an entire cohort of patients are graphically presented, wherein a first axis represents the level of the biomarker, and a second axis represents the number of patients in the cohort whose neoplastic cells express the biomarker at a given level.

Two or more separate groups of patients can be determined by identification of subset populations of the cohort which have the same or similar levels of the biomarker. Determination of the reference level can then be made based on a level which best distinguishes these separate groups. A reference level also can represent the levels of two or more markers. Two or more markers can be represented, for example, by a ratio of values for levels of each biomarker. The reference level can be a single number, equally applicable to every patient, or the reference level can vary, according to specific subpopulations of patients. For example, older individuals might have a different reference level than younger individuals for the same cancer. In another example, the reference level might be a certain ratio of a biomarker in the neoplastic cells of a patient relative to the biomarker levels in non-tumor cells within the same patient. Thus the reference level for each patient can be proscribed by a reference ratio of one or moregenomic markers, such as methylation of one or more genes, wherein the reference ratio can be determined by any of the methods for determining the reference levels described herein.

It is understood that the reference level has to correspond to the level of methylated genomic CpG dinucleotide sequences present in a corresponding sample that allows comparison to the desired phenotype. For example, in a diagnostic application a reference level can be based on a sample that is derived from a cancer-free origin so as to allow comparison to the biological test sample for purposes of diagnosis. In a method of staging a cancer it can be useful to apply in parallel a series of reference levels, each based on a sample that is derived from a cancer that has been classified based on parameters established in the art, for example, phenotypic or cytological characteristics, as representing a particular cancer stage so as to allow comparison to the biological test sample for purposes of staging. In addition, progression of the course of a condition can be determined by determining the rate of change in the level or pattern of methylation of genomic CpG dinucleotide sequences by comparison to reference levels derived from reference samples that represent time points within an established progression rate. It is understood, that the user will be able to select the reference sample and establish the reference level based on the particular purpose of the comparison.

As used herein, the term “neoplastic cell” refers to any cell that is transformed such that it proliferates without normal homeostatic growth control. Such cells can result in a benign or malignant lesion of proliferating cells. Such a lesion can be located in a variety of tissues and organs of the body. Exemplary types of cancers from which a neoplastic cell can be derived are set forth above.

As used herein, the term “cancer” is intended to mean a class of diseases characterized by the uncontrolled growth of aberrant cells, including all known cancers, and neoplastic conditions, whether characterized as malignant, benign, soft tissue or solid tumor. Specific cancers include digestive and gastrointestinal cancers, such as anal cancer, bile duct cancer, gastrointestinal carcinoid tumor, colon cancer, esophageal cancer, gallbladder cancer, liver cancer, pancreatic cancer, rectal cancer, appendix cancer, small intestine cancer and stomach (gastric) cancer; breast cancer; ovarian cancer; lung cancer; renal cancer; CNS 30 cancer; leukemia and melanoma. By exemplification, a list of known cancers is set forth above.

As used herein, the term “sample” is intended to mean any biological fluid, cell, tissue, organ or portion thereof, that contains genomic DNA suitable for methylation detection via the invention methods. A test sample can include or be suspected to include a neoplastic cell, such as a cell from the colon, rectum, breast, ovary, prostate, kidney, lung, blood, brain or other organ or tissue that contains or is suspected to contain a neoplastic cell. The term includes samples present in an individual as well as samples obtained or derived from the individual. For example, a sample can be a histologic section of a specimen obtained by biopsy, or cells that are placed in or adapted to tissue culture. A sample further can be a subcellular fraction or extract, or a crude or substantially pure nucleic acid molecule or protein preparation. A reference sample can be used to establish a reference level and, accordingly, can be derived from the source tissue that meets having the particular phenotypic characteristics to which the test sample is to be compared.

A sample may be obtained in a variety of ways known in the art. Samples may be obtained according to standard techniques from all types of biological sources that are usual sources of genomic DNA including, but not limited to cells or cellular components which contain DNA, cell lines, biopsies, bodily fluids such as blood, sputum, stool, urine, cerebrospinal fluid, ejaculate, tissue embedded in paraffin such as tissue from eyes, intestine, kidney, brain, heart, prostate, lung, breast or liver, histological object slides, and all possible combinations thereof. A suitable biological sample can be sourced and acquired subsequent to the formulation of the diagnostic aim of the marker. A sample can be derived from a population of cells or from a tissue that is predicted to be afflicted with or phenotypic of the condition. The genomic DNA can be derived from a high-quality source such that the sample contains only the tissue type of interest, minimum contamination and minimum DNA fragmentation. In particular, samples should be representative of the tissue or cell type of interest that is to be handled by the diagnostic assay. It is understood that samples can be analyzed individually or pooled depending on the purpose of the user. In addition, a population or set of samples from an individual source can be analyzed to maximize confidence in the results and can be a sample set size of 10, 15, 20, 25, 50, 75, 100, 150 or sample set sizes in the hundreds.

In subsequent steps of the method, the methylation levels of CpG positions are compared to a reference sample, to identify differentially methylated CpG positions. Each class may be further segregated into sets according to predefined parameters to minimize the variables between the at least two classes. In the following stages of the method, all comparisons of the methylation status of the classes of tissue, are carried out between the phenotypically matched sets of each class. Examples of such variables include, age, ethnic origin, sex, life style, patient history, drug response etc.

As used herein, the term “disease-free survival” refers to the lack of tumor recurrence and/or spread and the fate of a patient after diagnosis, for example, a patient who is alive without tumor recurrence.

The phrase “overall survival” refers to the fate of the patient after diagnosis, regardless of whether the patient has a recurrence of the tumor. As used herein, the term “risk of recurrence” refers to the probability of tumor recurrence or spread in a patient subsequent to diagnosis of cancer, wherein the probability is determined according to the process of the invention. Tumor recurrence refers to further growth of neoplastic or cancerous cells after diagnosis of cancer. Particularly, recurrence can occur when further cancerous cell growth occurs in the cancerous tissue. Tumor spread refers to dissemination of cancer cells into local or distant tissues and organs, for example during tumor metastasis. Tumor recurrence, in particular, metastasis, is a significant cause of mortality among patients who have undergone surgical treatment for cancer. Therefore, tumor recurrence or spread is correlated with disease free and overall patient survival.

The methods of the invention can be applied to the characterization, classification, differentiation, grading, staging, diagnosis, or prognosis of a condition characterized by a pattern of methylated genomic CpG dinucleotide sequences that is distinct from the pattern of methylated genomic CpG dinucleotide sequences exhibited in the absence of the condition. A condition that is suitable for practicing the methods of the invention can be, for example, cell proliferative disorder or predisposition to cell proliferative disorder; metabolic malfunction or disorder; immune malfunction, damage or disorder; CNS malfunction, damage or disease; symptoms of aggression or behavioural disturbance; clinical, psychological and social consequences of brain damage; psychotic disturbance and personality disorder; dementia or associated syndrome; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as an abnormality in the development process; malfunction, damage or disease of the skin, the muscles, the connective tissue or the bones; endocrine and metabolic malfunction, damage or disease; headache or sexual malfunction, and combinations thereof.

Methylation of CpG dinucleotide sequences can be measured using any of a variety of techniques used in the art for the analysis of specific CpG dinucleotide methylation status. For example, methylation can be measured by employing a restriction enzyme based technology, which utilizes methylation sensitive restriction endonucleases for the differentiation between methylated and unmethylated cytosines. Restriction enzyme based technologies include, for example, restriction digest with methylation-sensitive restriction enzymes followed by Southern blot analysis, use of methylation-specific enzymes and PCR, restriction landmark genomic scanning (RLGS) and differential methylation hybridization (DMH).

Restriction enzymes characteristically hydrolyze DNA at and/or upon recognition of specific sequences or recognition motifs that are typically between 4- to 8-bases in length. Among such enzymes, methylation sensitive restriction enzymes are distinguished by the fact that they either cleave, or fail to cleave DNA according to the cytosine methylation state present in the recognition motif, in particular, of the the CpG sequences. In methods employing such methylation sensitive restriction enzymes, the digested DNA fragments can be separated, for example, by gel electrophoresis, on the basis of size, and the methylation status of the sequence is thereby deduced, based on the presence or absence of particular fragments. Preferably, a post-digest PCR amplification step is added wherein a set of two oligonucleotide primers, one on each side of the methylation sensitive restriction site, is used to amplify the digested genomic DNA. PCR products are not detectable where digestion of the subtended methylation sensitive restriction enzyme site occurs. Techniques for restriction enzyme based analysis of genomic methylation are well known in the art and include the following: differential methylation hybridization (DMH) (Huang et al., Human Mol. Genet. 8, 459-70, 1999); Not I-based differential methylation hybridization (see e.g., WO 02/086163 A1); restriction landmark genomic scanning (RLGS) (Plass et al., Genomics 58:254-62, 1999); methylation sensitive arbitrarily primed PCR (AP-PCR) (Gonzalgo et al., Cancer Res. 57: 594-599, 1997); methylated CpG island amplification (MCA) (Toyota et. al., Cancer Res. 59: 2307-2312, 1999).

Methylation of CpG dinucleotide sequences also can be measured by employing cytosine conversion based technologies, which rely on methylation status-dependent chemical modification of CpG sequences within isolated genomic DNA, or fragments thereof, followed by DNA sequence analysis. Chemical reagents that are able to distinguish between methylated and non methylated CpG dinucleotide sequences include hydrazine, which cleaves the nucleic acid, and bisulfite treatment. Bisulfite treatment followed by alkaline hydrolysis specifically converts non-methylated cytosine to uracil, leaving 5-methylcytosine unmodified as described by Olek A., Nucleic Acids Res. 24:5064-6, 1996. The bisulfite-treated DNA can subsequently be analyzed by conventional molecular techniques, such as PCR amplification, sequencing, and detection comprising oligonucleotide hybridization.

Techniques for the analysis of bisulfite treated DNA can employ methylation-sensitive primers for the analysis of CpG methylation status with isolated genomic DNA as described by Herman et al., Proc. Natl. Acad. Sci. USA 93:9821-9826, 1996, and in U.S. Pat. Nos. 5,786,146 and 6,265,171. Methylation sensitive PCR (MSP) allows for the detection of a specific methylated CpG position within, for example, the regulatory region of a gene. The DNA of interest is treated such that methylated and non-methylated cytosines are differentially modified, for example, by bisulfite treatment, in a manner discernable by their hybridization behavior. PCR primers specific to each of the methylated and non-methylated states of the DNA are used in a PCR amplification. Products of the amplification reaction are then detected, allowing for the deduction of the methylation status of the CpG position within the genomic DNA. Other methods for the analysis of bisulfite treated DNA include methylation-sensitive single nucleotide primer extension (Ms-SNuPE) (Gonzalgo & Jones, Nucleic Acids Res. 25:2529-2531, 1997; and see U.S. Pat. No. 6,251,594), and the use of real time PCR based methods, such as the art-recognized fluorescence-based real-time PCR technique MethyLight.™. (Eads et al., Cancer Res. 59:2302-2306, 1999; U.S. Pat. No. 6,331,393 to Laird et al.; and see Heid et al., Genome Res. 6:986-994, 1996). It is understood that a variety of methylation assay methods can be used for the determination of the methylation status of particular genomic CpG positions. Methods which require bisulfite conversion include, for example, bisulfite sequencing, methylation-specific PCR, methylation-sensitive single nucleotide primer extension (Ms-SnuPE), MALDI mass spectrometry and methylation-specific oligonucleotide arrays and are described, for example, in U.S. patent application Ser. No. 10/309,803 and international application International Patent Application No.: PCT/US03/38582.

In one embodiment, methylation of genomic CpG positions in a sample can be detected using an array of probes. In particular embodiments, a plurality of different probe molecules can be attached to a substrate or otherwise spatially distinguished in an array. Exemplary arrays that can be used in the invention include, without limitation, slide arrays, silicon wafer arrays, liquid arrays, bead-based arrays and others known in the art or set forth in further detail below. In preferred embodiments, the methods of the invention can be practiced with array technology that combines a miniaturized array platform, a high level of assay multiplexing, and scalable automation for sample handling and data processing.

An array of arrays, also referred to as a composite array, having a plurality of individual arrays that is configured to allow processing of multiple samples can be used. Exemplary composite arrays that can be used in the invention are described in U.S. Pat. No. 6,429,027 and U.S. 2002/0102578 and include, for example, one component systems in which each array is located in a well of a multi-well plate or two component systems in which a first component has several separate arrays configured to be dipped simultaneously into the wells of a second component. A substrate of a composite array can include a plurality of individual array locations, each having a plurality of probes and each physically separated from other assay locations on the same substrate such that a fluid contacting one array location is prevented from contacting another array location. Each array location can have a plurality of different probe molecules that are directly attached to the substrate or that are attached to the substrate via rigid particles in wells (also referred to herein as beads in wells).

In a particular embodiment, an array substrate can be fiber optical bundle or array of bundles, such as those generally described in U.S. Pat. Nos. 6,023,540, 6,200,737 and 6,327,410; and PCT publications WO9840726, WO9918434 and WO9850782. An optical fiber bundle or array of bundles can have probes attached directly to the fibers or via beads. Other substrates having probes attached to a substrate via beads are described, for example, in US 2002/0102578. A substrate, such as a fiber or silicon chip, can be modified to form discrete sites or wells such that only a single bead is associated with the site or well. For example, when the substrate is a fiber optic bundle, wells can be made in a terminal or distal end of individual fibers by etching, with respect to the cladding, such that small wells or depressions are formed at one end of the fibers. Beads can be non-covalently associated in wells of a substrate or, if desired, wells can be chemically functionalized for covalent binding of beads. Other discrete sites can also be used for attachment of particles including, for example, patterns of adhesive or covalent linkers. Thus, an array substrate can have an array of particles each attached to a patterned surface.

In a particular embodiment, a surface of a substrate can include physical alterations to attach probes or produce array locations. For example, the surface of a substrate can be modified to contain chemically modified sites that are useful for attaching, either-covalently or non-covalently, probe molecules or particles having attached probe molecules. Chemically modified sites can include, but are not limited to the linkers and reactive groups set forth above. Alternatively, polymeric probes can be attached by sequential addition of monomeric units to synthesize the polymeric probes in situ. Probes can be attached using any of a variety of methods known in the art including, but not limited to, an ink-jet printing method as described, for example, in U.S. Pat. Nos. 5,981,733; 6,001,309; 6,221,653; 6,232,072 or 6,458,583; a spotting technique such as one described in U.S. Pat. No. 6,110,426; a photolithographic synthesis method such as one described in U.S. Pat. No. 6,379,895 or 5,856,101; or printing method utilizing a mask as described in U.S. Pat. No. 6,667,394. Accordingly, arrays described in the aforementioned references can be used in a method of the invention.

The size of an array used in the invention can vary depending on the probe composition and desired use of the array. Arrays containing from about 2 different probes to many millions can be made. Generally, an array can have from two to as many as a billion or more probes per square centimeter. Very high density arrays are useful in the invention including, for example, those having from about 10,000,000 probes/cm² to about 2,000,000,000 probes/cm² or from about 100,000,000 probes/cm² to about 1,000,000,000 probes/cm². High density arrays can also be used including, for example, those in the range from about 100,000 probes/cm² to about 10,000,000 probes/cm² or about 1,000,000 probes/cm² to about 5,000,000 probes/cm². Moderate density arrays useful in the invention can range from about 10,000 probes/cm² to about 100,000 probes/cm², or from about 20,000 probes/cm² to about 50,000 probes/cm². Low density arrays are generally less than 10,000 probes/cm² with from about 1,000 probes/cm² to about 5,000 probes/cm² being useful in particular embodiments. Very low density arrays having less than 1,000 probes/cm², from about 10 probes/cm² to about 1000 probes/cm², or from about 100 probes/cm² to about 500 probes/cm² are also useful in some applications.

Thus, the invention provides a robust and ultra high-throughput technology for simultaneously measuring methylation at many specific sites in a genome. The invention further provides cost-effective methylation profiling of thousands of samples in a reproducible, well-controlled system. In particular the invention allows implementation of a process, including sample preparation, bisulfite treatment, genotyping-based assay and PCR amplification that can be carried out on a robotic platform.

The methods of the invention can be carried out at a level of multiplexing that is 96-plex or even higher including, for example, as high as 1,500-plex. An advantage of the invention is that the amount of genomic DNA used for detection of methylated sequences is low including, for example, less that 1 ng of genomic DNA per locus. In one embodiment, the throughput of the methods can be 96 samples per run, with 1,000 to 1,500 methylation assays per sample (144,000 data points or more per run). In the embodiment exemplified herein, the system is capable of carrying out as many as 10 runs per day or more. A further object of the invention is to provide assays to survey methylation status the 5′-regulatory regions of at least 1,000 human genes per sample. Particular genes of interest are tumor suppressor genes or other cancer-related genes, as well as genes identified through RNA profiling.

Therefore, the invention makes available diagnostic and/or prognostic assays for the analysis of the methylation status of CpG dinucleotide sequence positions as markers for disease or disease-related conditions. As described herein, the invention provides a systematic method for the identification, assessment and validation of genomic targets as well as a systematic means for the identification and verification of multiple condition relevant CpG positions to be used alone, or in combination with other CpG positions, for example, as a panel or array of markers, that form the basis of a clinically relevant diagnostic or prognostic assay. The inventive method enables differentiation between two or more phenotypically distinct classes of biological matter and allows for the comparative analysis of the methylation patterns of CpG dinucleotides within each of the classes.

A further object of the invention is to provide assays for specific identifying methylation patterns in different cancer types and cancer stages. A further object of the invention is to provide software to retrieve and annotate CpG island sequence information, design and analyze primers, track sample information, and analyze and report results obtained from methylation profiling methods of the invention. An advantage of the invention is that it provides a high throughput methylation analysis system that can be commercialized, both through a service business—in which customers can provide samples and a gene list (CpG site list) for analysis in the methods—and through products that can used in standard laboratory conditions.

RLGS profiling of the methylation pattern of 1184 CpG islands in 98 primary human tumors revealed that the total number of methylated sites is variable between and in some cases within different tumor types, suggesting there may be methylation subtypes within tumors having similar histology (Costello, et al., Nat Genet. 24(2): 132-8 (2000)). Aberrant methylation of some of these genes correlates with loss of gene expression. Based on these observations, it should be feasible to use the methylation pattern of a sizable group of tumor suppressor genes or other cancer-related genes to classify and predict different kinds of cancer, or the same type of cancer in different stages. It promises to provide a useful tool for cancer diagnosis, or preferably, for detection of premalignant changes. When combined with the development of sensitive, non-invasive methods (e.g. a blood test; indeed, circulating tumor nucleic acids in blood have been demonstrated to reflect the biologic characteristics of tumors (Cui, et al., Science 299 (5613): 1753-5 (2003)), to detect such methylation signatures) this may provide a viable method to screen subjects at risk for cancer as well as to monitor cancer progression and response to treatment.

Because methylation detection interrogates genomic DNA, but not RNA or protein, it offers several technological advantages in a clinical diagnostic setting: (1) readily available source materials. This is particularly important for prognostic research, when only DNA can be reliably extracted from archived paraffin-embedded samples for study; (2) capability for multiplexing, allowing simultaneous measurement of multiple targets to improve assay specificity; (3) easy amplification of assay products to achieve high sensitivity; (4) robust measurement in tumors that arise from methylation inactivation of one allele of tumor suppressor genes—a process called “functional haploinsufficiency” (Balmain, et al., Nat Genet. 33 Suppl: 238-44 (2003)). It is much easier to detect a methylation change (from negative to positive) than to detect a two-fold gene expression change in these tumors. In summary, when combined with RNA-based gene expression profiling and/or protein-based immunoassays, DNA methylation profiling should provide a sensitive, accurate and robust tool for cancer diagnosis and prognosis (Wong, et al., Curr Oncol Rep. 4(6): 471-7 (2002)).

The present invention is directed to a method for the identification of differentially methylated CpG dinucleotides within genomic DNA that are particularly informative with respect to disease states. These may be used either alone or as components of a gene panel in diagnostic and/or prognostic assays.

In particular embodiments, the invention is directed to methods of prediction and diagnosis of conditions characterized by a pattern of methylated genomic CpG dinucleotide sequences that is distinct from the pattern of methylated genomic CpG dinucleotide sequences exhibited in the absence of the particular condition, for example, cell proliferative disorders, such as cancer; dysfunctions, damages or diseases of the central nervous system (CNS), including aggressive symptoms or behavioral disorders; clinical, psychological and social consequences of brain injuries; psychotic disorders and disorders of the personality, dementia and/or associates syndromes; cardiovascular diseases, malfunctions or damages; diseases, malfunctions or damages of the gastrointestine diseases; malfunctions or damages of the respiratory system; injury, inflammation, infection, immunity and/or reconvalescence, diseases; malfunctions or damages as consequences of modifications in the developmental process; diseases, malfunctions or damages of the skin, muscles, connective tissue or bones; endocrine or metabolic diseases malfunctions or damages; headache; and sexual malfunctions; or combinations thereof.

It is understood that modifications which do not substantially affect the activity of the various embodiments of this invention are also included within the definition of the invention provided herein. Accordingly, the following examples are intended to illustrate but not limit the present invention.

### EXAMPLE I

### Design of Target Nucleic Acid Probes

This Example shows design of target nucleic acid probes for detection of genomic loci.

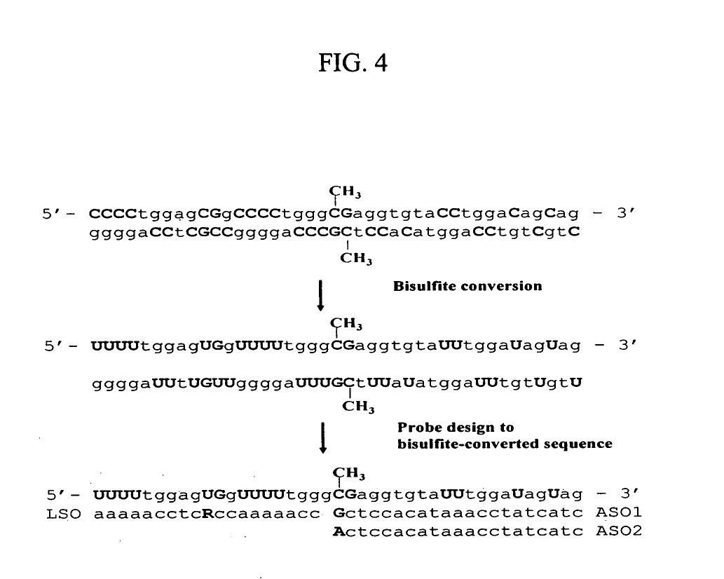

First, a human gene promoter database was prepared that includes all CpG regions of potential interest for methylation profiling. A fully automated SNP genotyping assay design program was adapted for methylation application and CpG islands of interest are selected and “converted by bisulfite” computationally. For each CpG locus, three probes are designed: two allele-specific oligonucleotides, one corresponding to the methylated, and the other to the unmethylated state of the CpG site and one locus-specific oligo (FIG. 4). If other CpG loci are present in the close vicinity of the chosen CpG site, a wobble base [A or G] is used for the corresponding probe position. Assays for more than 60 CpG sites from 20 different genes were designed, mostly selected from the methylation database on the world-wide-web at methdb.de. Approximately half of the sites were used in the assay development.

### EXAMPLE II

### Development of Internal Controls and Confirmation of Completeness of Bisulfite Conversion

This example shows the development of internal controls that allow optimization of protocols, determination of assay specificity, troubleshooting, and evaluation of overall assay performance.

Plasmids pUC19, pACYC184 and phage phiX174 were selected to serve as control DNAs. These DNAs can be spiked into the genomic DNA assays to provide internal controls, and would not interfere with human genomic DNA reactions. It is easy to prepare completely unmethylated plasmid DNAs and then methylate them in vitro using Sss I (CpG) methylase to produce substrates with known methylation status. Plasmids can be methylated virtually to completion. The quality of in vitro methylation was tested by restriction enzyme digestion of unmethylated and methylated DNAs using the methylation sensitive enzyme Hpa II and its isoschisomer Msp I, which is not sensitive to methylation. It was not possible to detect any bands on the agarose gel after incubation of methylated pUC19, pACYC184 and phiX174 with Hpa II for two hours at 37° C., while the unmethylated DNAs were completely digested. Both methylated and unmethylated DNAs were completely digested by Msp I.

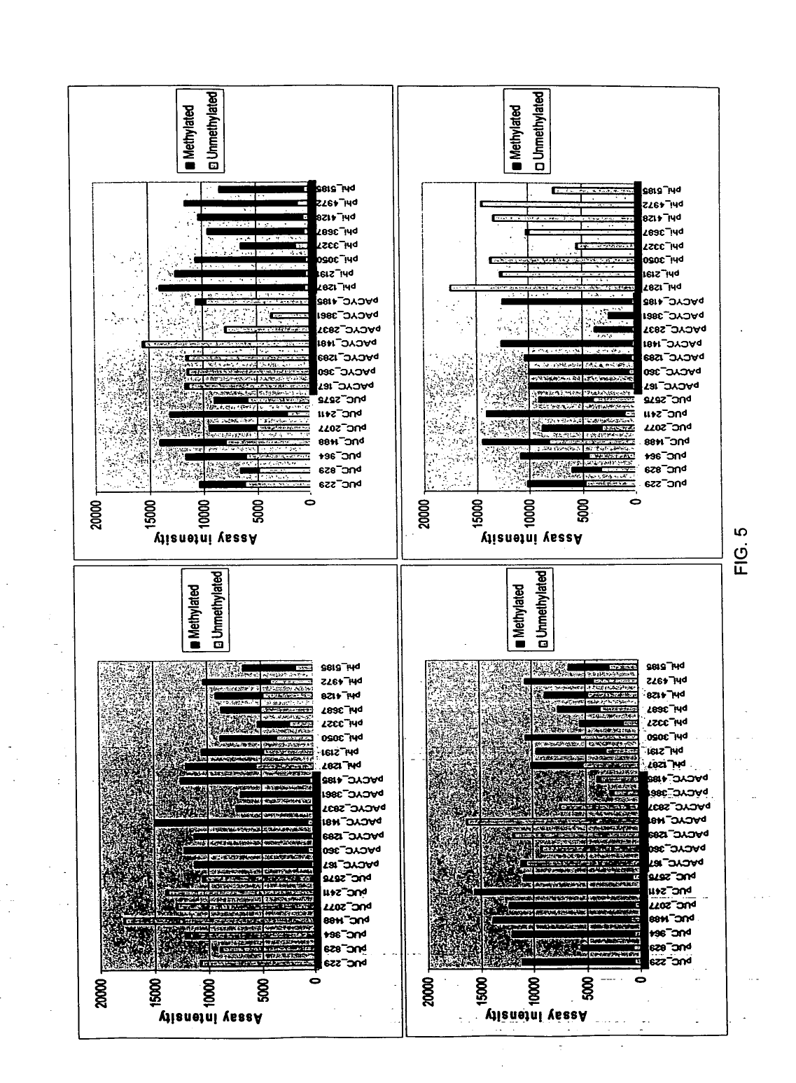

Plasmid controls (unmethylated, methylated or mixed at a 1:1 ratio) were spiked into human genomic DNA at a 1:1 molar ratio (at approximately 2-4 pg plasmid DNA/1 μg gDNA, depending on the plasmid size), and were used in every methylation experiment to monitor both bisulfite conversion efficiency and accuracy of methylation detection. As shown in FIG. 5, unmethylated, semi-methylated and fully-methylated loci can be easily distinguished by the assay.

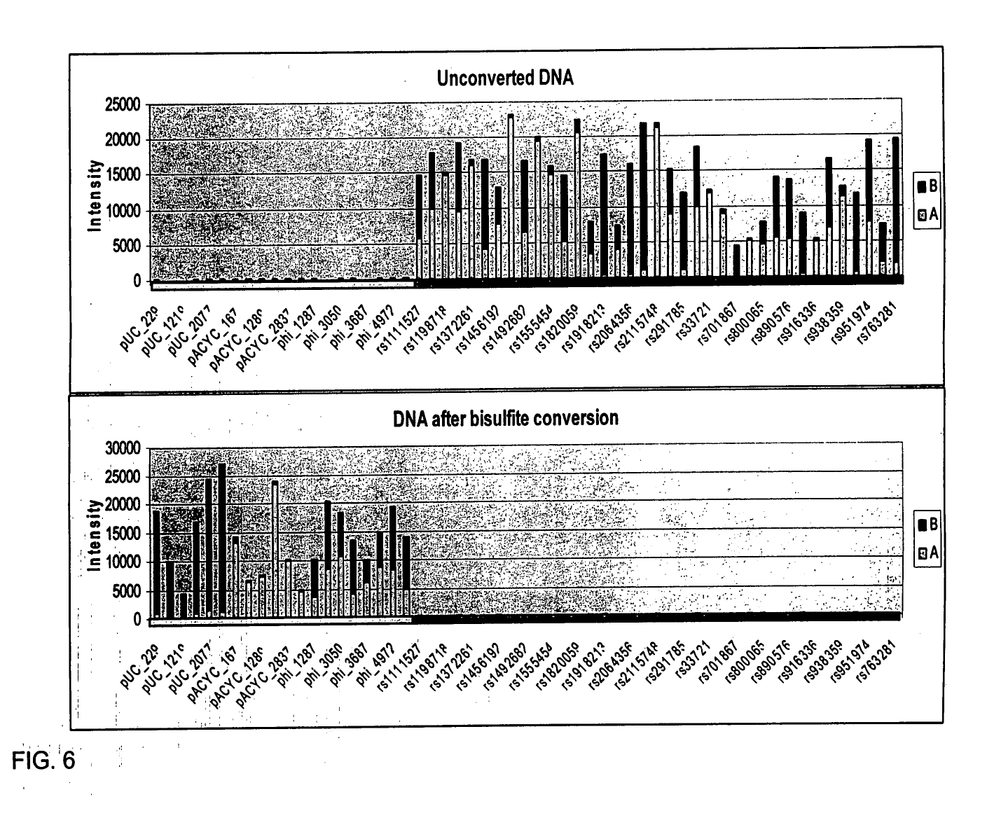

The utility of bisulfite conversion of DNA for methylation detection is based on the different sensitivity of cytosine and 5-methylcytosine to deamination by bisulfite. Under acidic conditions, cytosine undergoes conversion to uracil, while methylated cytosine remains unreactive. An efficient bisulfite conversion protocol is a necessary prerequisite for a high-throughput methylation profiling assay. Incomplete conversion of cytosine to uracil by bisulfite can result in appearance of false-positive signals for 5-methylcytosine, and reduce the overall quality of the assay data. In order to monitor the effectiveness of bisulfite treatment, a set of oligonucleotides (a standard set of SNP genotyping probes) designed for unconverted genomic DNA sequences was included with the plasmid control oligos in the assay. As shown in FIG. 6, if bisulfite conversion is successful, the signal from oligonucleotides targeted to the unconverted DNA (the SNP set) will disappear, and signals from oligonucleotides targeted to the converted DNA will be present. Incomplete conversion will result in low and inconsistent signals across all targeted loci.

Data Processing

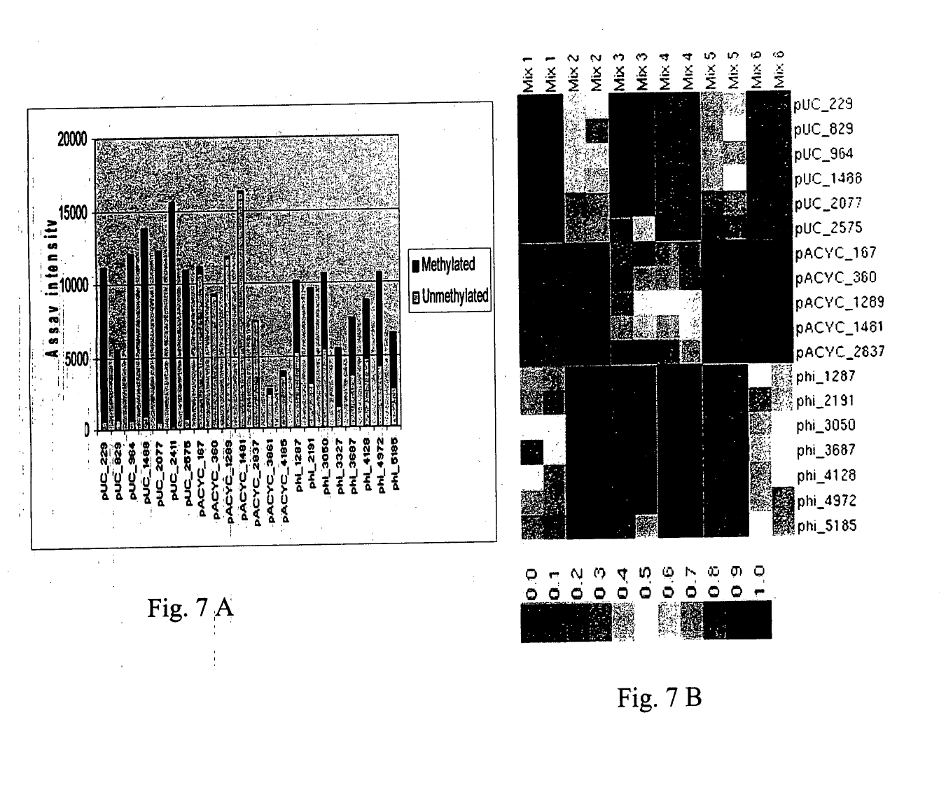

Development of a robust and high-throughput method for simultaneous measurement of methylation at many specific sites in many samples requires a highly efficient analysis and data export process. Each data point in the methylation assay can be represented as a ratio of the fluorescent signals from M (methylated) and U (unmethylated) specific PCR products after array hybridization. This value indicates the methylation status of the CpG locus and may range from 0 in the case of completely unmethylated sites to 1 in completely methylated sites. The value also can be visually presented as a red/green/yellow plot (FIG. 7). In addition, each locus is characterized by a locus intensity value, which allows filtering of failed loci. This combination of numerical and color outputs allows for quick comparison of genes and samples of interest, and processing of thousands of loci across hundreds of samples.

Reference Samples for Genome-Wide Methylation Profiling

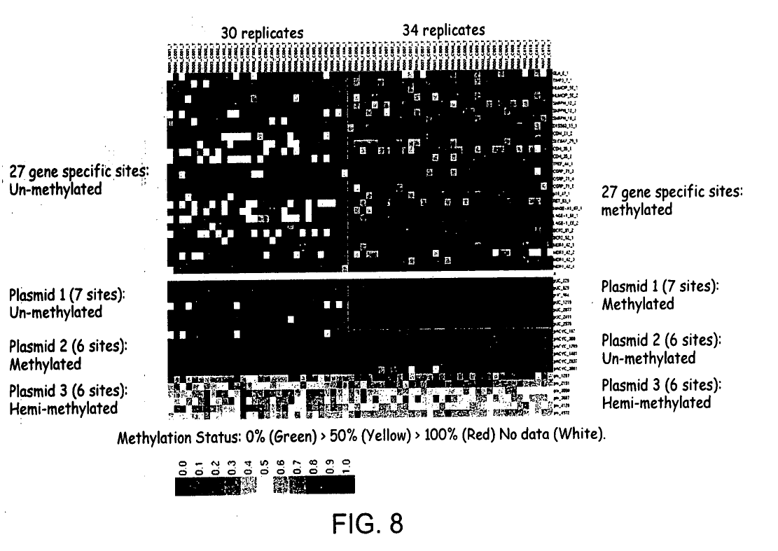

To calibrate quantitative measurements of methylation, “fully methylated” and “unmethylated” genomic templates were developed (FIG. 8). The fully unmethylated templates were generated by genome-wide amplification of human genomic DNA. With more than 1000-fold amplification, any endogenous methylation is effectively “diluted/erased”, and the sample can be used as an “unmethylated” reference DNA. Methylated templates were generated by in vitro methylation of amplified gDNA using Sss I (CpG-methylase) enzyme. The reproducibility of the methylation detection method was confirmed by typing 27 human gene-specific CpG sites in these reference DNAs over 30 times. A high degree of reproducibility was obtained (FIG. 8).

Methylation Profiles of Randomly Selected Human Genomic DNAs

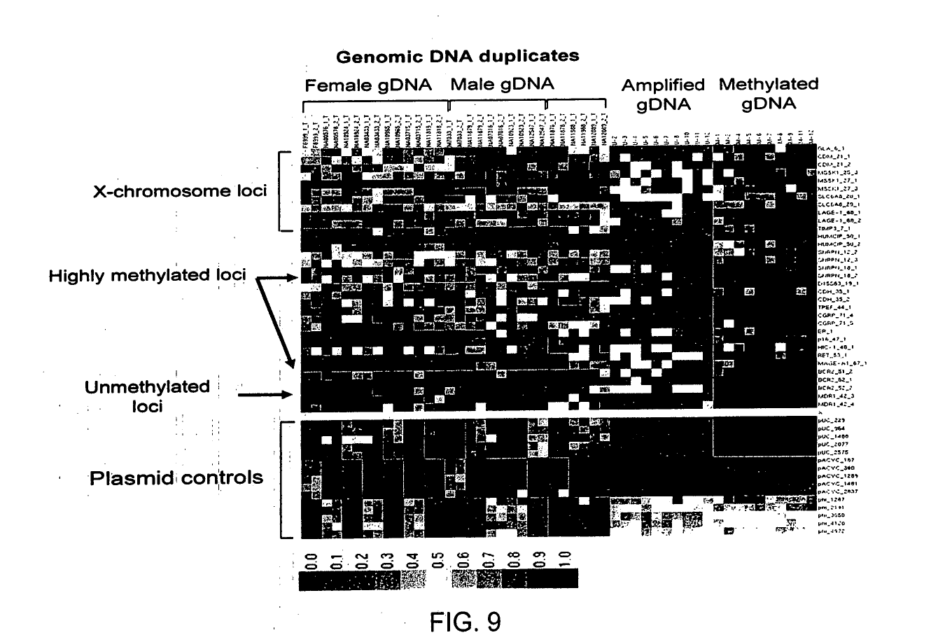

We have also monitored a set of CpG sites in randomly selected human genomic DNAs. The DNA samples were obtained from the Human Genetic Cell Repository, Coriell Institute for Medical Research, NJ. In this experiment, we measured 7 females, 5 males and 3 of unknown gender specificity; each was done in duplicate and the results are shown in columns next to each other in FIG. 9. Distinguishable methylation patterns were obtained with DNAs isolated from male and female cell lines, especially in genes that are located on the X-chromosome (FIG. 9). The overall methylation profiles are quite reproducible among the duplicates.

Assay Reproducibility

One of the most important assay characteristics is its reproducibility. This was addressed by monitoring several plasmid loci with known methylation status across multiple replicates in several independent experiments. The experimental setup also allowed for estimation of the assay accuracy. A mixture of unmethylated, methylated and 1:1 mixed plasmid DNAs was prepared, and spiked into 1 μg of human genomic DNA in a 1:1 molar ratio. Each experiment included three plasmid mixtures:

| | | | |
|---|---|---|---|
| 1 | pUC19 U | pACYC 184 M | phiX174 U:M |
| 2 | pUC19 U:M | pACYC 184 U | phiX174 M |
| 3 | pUC19 M | pACYC 184 U:M | phiX174 U |

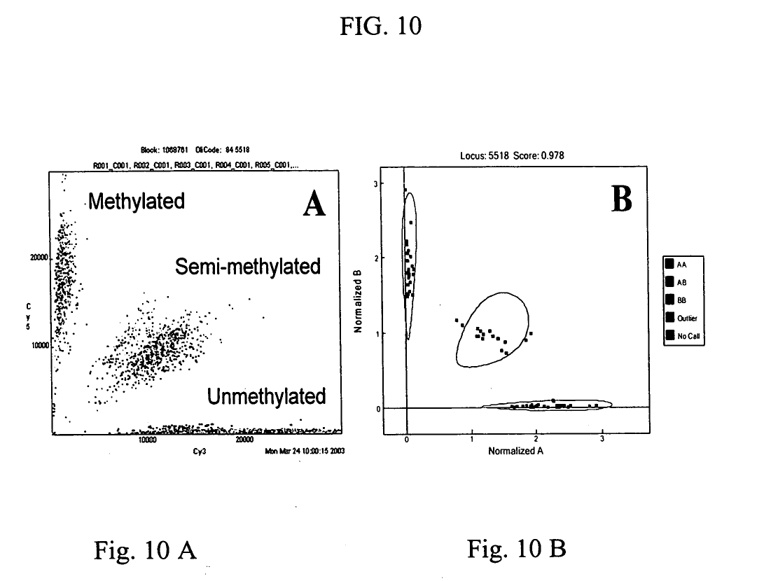

A typical experiment included 32 replicates of each set of the three plasmid mixtures assayed on a 96 fiber bundle array matrix. Results of the reproducibility study are summarized in Table 2, which involve (79+96+95+95) replicates ×14 CpG sites =5,110 measurements. It is noticeable that some loci (e.g. phi4972) tend to perform better than others (e.g. pACYC_{—}360). There are also some performance variations from experiment to experiment. The overall call accuracy is averaged at ˜97% with a high call rate of 99.6%. The accuracy was calculated using our existing SNP genotyping software, which uses a clustering algorithm to determine if a locus is methylated, unmethylated or semi-methylated (FIG. 10).

TABLE 4
Summary of methylation measurement with 14 plasmid CpG loci.

| | Experiment 1, | | | Experiment 2, | | | Experiment 3, | | |
|---|---|---|---|---|---|---|---|---|---|
| | 79 replicates | | | 96 replicates | | | 95 replicates | | |
| | | | Wrong | | | Wrong | | | Wrong |
| Locus | No call, % | Correct, % | call, % | No call, % | Correct, % | call, % | No call, % | Correct, % | call, % |
| pUC_229 | 0 | 100 | 0 | 0 | 93.75 | 6.25 | 0 | 93.68 | 6.32 |
| pUC_964 | 0 | 100 | 0 | 0 | 89.58 | 10.42 | 0 | 94.74 | 5.26 |
| pUC_1488 | 1.27 | 100 | 0 | 0 | 96.88 | 3.13 | 0 | 91.58 | 8.42 |
| pUC_2077 | 0 | 100 | 0 | 8.33 | 88.64 | 11.36 | 6.32 | 91.01 | 8.99 |
| pUC_2575 | 0 | 100 | 0 | 0 | 88.54 | 11.46 | 0 | 90.53 | 9.47 |
| pACYC_167 | 0 | 100 | 0 | 0 | 88.54 | 11.46 | 0 | 97.89 | 2.11 |
| pACYC_360 | 0 | 98.73 | 1.27 | 1.04 | 74.74 | 25.26 | 0 | 91.58 | 8.42 |
| pACYC_1289 | 0 | 100 | 0 | 1.04 | 89.47 | 10.53 | 0 | 95.79 | 4.21 |
| pACYC_1481 | 0 | 100 | 0 | 1.04 | 92.63 | 7.37 | 0 | 95.79 | 4.21 |
| phi_2191 | 0 | 100 | 0 | 0 | 98.96 | 1.04 | 0 | 95.79 | 4.21 |
| phi_3050 | 0 | 100 | 0 | 0 | 100 | 0 | 0 | 98.95 | 1.05 |
| phi_3687 | 0 | 97.47 | 2.53 | 2.08 | 94.68 | 5.32 | 3.16 | 92.39 | 7.61 |
| phi_4128 | 0 | 100 | 0 | 0 | 100 | 0 | 0 | 98.95 | 1.05 |
| phi_4972 | 0 | 100 | 0 | 0 | 100 | 0 | 0 | 100 | 0 |
| Total: | 0.09 | 99.73 | 0.27 | 0.97 | 92.60 | 7.40 | 0.68 | 94.90 | 5.10 |

| | Experiment 4, | | | | | | |
|---|---|---|---|---|---|---|---|
| | 95 replicates | | | Summary | | | |
| | | | Wrong | | Wrong | | Correct, |
| Locus | No call, % | Correct, % | call, % | No call, % | call, % | Correct, % | CV (%) |
| pUC_229 | 0 | 100 | 0 | 0 | 3.14 | 96.86 | 3.75 |
| pUC_964 | 0 | 100 | 0 | 0 | 3.92 | 96.08 | 5.20 |
| pUC_1488 | 0 | 100 | 0 | 0.32 | 2.89 | 97.11 | 4.09 |
| pUC_2077 | 0 | 96.84 | 3.16 | 3.66 | 5.88 | 94.12 | 5.55 |
| pUC_2575 | 0 | 100 | 0 | 0 | 5.23 | 94.77 | 6.43 |
| pACYC_167 | 0 | 100 | 0 | 0 | 3.39 | 96.61 | 5.66 |
| pACYC_360 | 0 | 97.89 | 2.11 | 0.26 | 9.26 | 90.73 | 12.27 |
| pACYC_1289 | 0 | 97.89 | 2.11 | 0.26 | 4.21 | 95.79 | 4.75 |
| pACYC_1481 | 0 | 100 | 0 | 0.26 | 2.89 | 97.11 | 3.69 |
| phi_2191 | 0 | 100 | 0 | 0 | 1.31 | 98.69 | 2.02 |
| phi_3050 | 0 | 98.95 | 1.05 | 0 | 0.53 | 99.47 | 0.61 |
| phi_3687 | 0 | 97.89 | 2.11 | 1.31 | 4.39 | 95.61 | 2.69 |
| phi_4128 | 0 | 98.95 | 1.05 | 0 | 0.53 | 99.47 | 0.61 |
| phi_4972 | 0 | 100 | 0 | 0 | 0 | 100 | 0 |
| Total: | 0 | 99.17 | 0.83 | 0.43 | 3.40 | 96.60 | 4.09 |

Note: Experiment 1 included 80 replicates of bisulfite converted DNA and 16 replicates of unconverted samples for background control. The other 3 experiments included only bisulfite converted DNA.

As demonstrated above, assay sensitivity and specificity were shown to be sufficient to detect changes in methylation status at more than 50 loci simultaneously in 1 microgram of human genomic DNA. A minimum of three levels of methylation was clearly distinguished: fully methylated, hemi-methylated, and unmethylated. The ability to distinguish three levels of methylation was confirmed by using plasmid control DNAs with known methylation status, spiked into human genomic DNA in a 1:1 molar ratio. Furthermore, reproducibility of methylation determination was shown to be 96.6% (which is a more stringent measurement than reproducibility), at a call rate exceeding 90% (Table 2). A set of three reference samples for 14 CpG sites was analyzed in four independent experiments. The number of measurements in each experiment was 1 106, 1344, 1330 and 1330 respectively.

Overall, this example demonstrates the development of a microtiter plate based, high throughput bisulfite conversion, which as described in the following Example, can be fully integrated into the SNP genotyping system for high-throughput methylation profiling. The methylation assays can be enlarged in both the scope and capacity of methylation detection with as many as 1500 methylation sites in each assay, while using reduced amounts of genomic DNA. Since the data collection and processing are largely automated, it is possible to do at least ten array matrix runs per day per system, with each run providing data from 96 samples at a time, creating a highly scalable system where multiple instruments can be run in parallel if needed.

### EXAMPLE III

### Integration of Microtiter Plate Based, Higfh-Throughput Bisulfite Conversion into Genotyping System for High Troughput Methylation Profiling

This example demonstrates the integration of a microtiter plate based, high throughput bisulfite conversion as described in Example II, into the SNP genotyping system for high-throughput methylation profiling.

The assay optimization process includes measuring the array-to-array experimental variability, both within a matrix and between matrices, and dissect out contributions to variability from samples, sample processing (bisulfite conversion, allele-specific extension, ligation, and PCR amplification), and array hybridization, using carefully designed controls. The resulting data also is useful in determining thresholds of significance for analyzing and interpreting results.

Improve Assay Performance with Fully “Methylated” and “Un-Methylated” Genomic Templates

Currently, all the methods used to validate methylation status of any methylation site in any given sample, such as bisulfite sequencing and methylation-specific PCR, are either laborious and time consuming, or inaccurate and expensive. Indeed, a high performance methylation quantitation (or calibration) system is needed for large-scale genome-wide methylation assay development and validation. As described in Example II, a system has been developed that uses fully “methylated” and “un-methylated” genomic templates.

The un-methylated templates can be generated by genome-wide amplification of any genomic DNA, using random primed DNA amplification with enzymes such as Phi-29, Taq DNA polymerase or Klenow Fragment (3′→5′-exo-). After this amplification, the endogenous DNA methylation is diluted at least 100 to 1000-fold, effectively rendering the amplified genome DNA “un-methylated”.

The methylated templates can be generated by in vitro methylation using the SssI CpG-methylase. However, not all the CpG sites can be fully methylated in vitro. Some of these can result from base substitution at the CpG sites in the DNA tested, in particular, these sites become “methylation-resistant”. It is well known that CpG sites are mutation hot spots. In order to achieve higher levels of genomic DNA methylation, different experimental conditions are tested, for example, varying the concentration of magnesium in the methylation reaction and using multiple methylases.

The above-described templates are used for assay development and calibration. The fully methylated and unmethylated genomic DNA templates can be mixed at different ratio, for example, 0%, 25%, 50%, 75%, and 100% of methylated template. Methylation assays on these mixed templates generate a calibration curve for quantitative methylation measurement in unknown samples for any CpG site in the genome. The mixed.templates can also be used to determine the sensitivity of methylation detection, for example, what percentage of the methylated template can be detected in the presence of un-methylated template.

Since this approach can be used to evaluate the methylation assay designed for any specific CpG site across the entire genome, it greatly aids methylation assay development such as assay specificity. For example, if an assay gives the same methylation “report” for both the unmethylated and the methylated DNA templates, it confirms that the assay is not working properly.

Finally, protocols for measuring DNA methylation in formalin-fixed, paraffin-embedded samples are created. Robust large-scale DNA methylation detection on these samples opens up a huge sample resource, for which clinical history is already available.

Improvement of Assay Sensitivity by Genomic DNA Amplification after Bisulfite Conversion

In order to improve assay sensitivity, DNA is amplified after bisulfite conversion, using a random priming approach. But, instead of using all possible random primers, advantage is taken of the unique sequence feature of genomic DNAs after bisulfite treatment, i.e. that un-methylated cytosines are converted to uracil. Therefore, these DNA templates contain mostly three bases, A, G and T (and U). The genomic amplification is carried out using (i) through (iii) as set forth in the following paragraphs.

(i) A mixture of two sets of primers that contain all possible combinations of three nucleotides: (i.e. A, T and C for one set, and A, T and G for the other set). Primers from the first set have higher affinity to the original bisulfite converted DNA strand, while primers from the second set preferentially anneal to the newly synthesized complementary strand. Using this scheme, having G and C in the same primer is avoided, thus preventing the primers from crossing over any CpG sites to be interrogated. Bias that may be introduced by the un-balanced annealing efficiency of primers corresponding to the two alleles (C or T) also is avoided. Lastly, since each primer set contains all possible combinations of three, but not four nucleotides, effective primer concentration is increased.

(ii) Simple primer sequences that contain only Adenines (A). The homopoly-A primers (for example, 6-mer, 9-mer, or longer) is used for the first strand synthesis. After that, a homopoly-T tail is added to the 3′-ends of the first strand products, using terminal deoxyribonucleotide transferase (TdT). A standard PCR is then be carried out to amplify the DNAs using a poly-A primer. Human chromosome 1 sequence was used to calculate the poly-T frequencies in the genome after bisulfite conversion: on average, the physical distance between any two poly-(T)n sequences (n>=9) is 330 bp, a perfect amplicon size range for robust amplifications.

(iii) Similar to approach (i), except that oligo-A primer is used for the first strand synthesis and primers containing combinations of A, T and G for the complementary strand synthesis.

Probe design is one of the critical components for a successful methylation assay. The fully automated SNP genotyping assay design program described herein can be used for methylation assay development. As shown above, for each CpG locus, three probes are designed: two allele-specific oligonucleotides, one corresponding to the methylated and the other to the unmethylated state of the CpG site, and one locus-specific oligo. If other CpG loci are present close to the chosen CpG site, a wobble base [A or G] is used in the corresponding position of the probes. Based on the observation that adjacent CpG sites tend to be co-methylated or co-de-methylated, a simpler design scheme is applied in which a “CG” sequence is used for all the CpG sites within the vicinity of the design, in particular for the landing sites for both ASO and LSO, to target any methylated CpG site; while a “TG” sequence is used for all the CpG sites within the vicinity of the design, to target any un-methylated CpG site. This approach requires two LSO oligos for some loci, but adds better discrimination between the methylated and unmethylated alleles.

A human gene promoter database, which includes all CpG regions of potential interest for methylation profiling was constructed by combining NCBI's RefSeq annotation, existing knowledge of some well-studied promoters, gene and promoter prediction algorithms, as well as observations of certain cancer-related genes. This database is continuously expanded by integrating more public information from literature and databases, and experimental observations. For the methylation study, a new database searching strategy is integrated into the primer design software. A modified genome database is generated in which all “C”s (except those located within a CpG dinucleotide sequence) are converted to “T”s in silico. The probe design program searches against this converted database to find unique sequences and compute melting temperature (Tm), self-complementarity and length for an optimal probe. An optimization program is applied to match address sequences with locus-specific oligos to minimize self-complemeniarities of combined address and probe sequences. A locus filtering program is used to filter out sequences predicted to be unsuitable on the basis of data from SNP genotyping experiments already carried out. Some sequence features have been shown to be troublesome, e.g. runs of six or more consecutive bases of a single type, extreme GC or AT content, inverted repeats (mostly due to secondary structure), and high numbers of hits in the human genome sequence based on similarity searches by BLAST. All of these parameters can be computed in advance. These parameters are stored in a relational database for further data analysis. In addition, the program computes the sequence complementarity between the probes designed for a given set of methylation sites, especially the sequence complementary at their 3′ends. This calculation allows assessment of the compatibility of the assays, which in turn provides guidance regarding grouping of the assays properly for multiplexing.

The main problem for the methylation assay primer design lies in the reduced complexity of the genome after bisulfite conversion of the genomic DNA. Analysis of 5′-regulatory sequences from 1200 human genes was performed and preliminary computer simulation analysis indicates that the length of the primers designed for the bisulfite-converted DNAs will have to be increased by several bases as compared to the un-converted ones to achieve the same primer specificity and melting temperature. If necessary, longer primers along with increased assay stringency is used.

In another in-silico experiment, three BLAST searchable human genomic sequence databases were created. The first database mimics methylated condition (after bisulfite treatment), where all the C residues in CpG dinucleotide sequence remains as C; the second mimics un-methylated conditions, where all the C residues in CpG dinucleotide sequence are converted into T; both of these databases have C residues from non-CpG sites converted into T. The third database has normal genomic sequences. BLAST searching against these databases using designed probes as queries was performed and, as predicted, the probes had much larger number of hits to the database that have “C” converted into “T” (the first two databases), and less number of hits to the normal database (the third database). Subsequent empirical experiments with probes that have either large or small number of BLAST hits suggested that the probes with small number of hits usually generate good assay results, while the probes with large number of hits do not. In the future, this BLAST search process is automated and integrated into the probe design software. Furthermore, probes is designed for all the CpG sites in the promoter regions. After a subsequent BLAST filtering process, only three probes are synthesized for each gene. For a small number of genes that can't have three qualified probes designed due to limited number of CpG sites in the promoter region or CpG sites too close to each other, or/and severe sequence similarity to other genomic regions.

In order to search for specific methylation patterns in different cancer types or cancer stages, we first develop methylation assays for at least 1000 human genes. These genes are selected based on the following criteria:

(i) Biological relevance. Methylation patterns in previously characterized tumor suppressor genes and oncogenes are an initial focus (Esteller, Oncogene 21(35): 5427-40 (2002), Adorjan, et al., Nucleic Acids Res. 30(5): p. e21 (2002)). Then, the target group is enlarged to include genes that are indirectly involved in cancer development, for example, DNA repair genes; metastasis-inhibitor genes, genes regulated by various signaling pathways, and/or responsible for altered cell growth and differentiation; or genes considered to be targets for oncogenic transformation.

(ii) Previous knowledge, e.g. genes located within published recurrent loss of hetrozygosity (LOH) regions or amplified genomic regions (Pollack, et al., Proc Natl Acad Sci U S A. 99(20): 12963-8 (2002)).

(iii) Gene expression profiling information, for example, genes differentially expressed in cancer and normal tissues. In the past few years, due to the rapid development of microarray technology, many specific gene-expression signatures have been identified for different cancer types (Golub, et al., Science 286(5439): 531-7 (1999), Ramaswamy, et al., Proc Natl Acad Sci U S A. 98(26): 15149-54 (2001), Perou, et al., Nature 406(6797): 747-52 (2000), Bhattacharjee, et al., Proc Natl Acad Sci U S A 98(24): 13790-5 (2001), Chen, et al., Mol Biol Cell 13(6): 1929-39 (2002), Welsh, et al., Proc Natl Acad Sci U S A. 98(3): 1176-81 (2001)), cancer stages (Dhanasekaran, et al., Nature 412(6849): 822-6 (2001), Ramaswamy, et al., Nat Genet. 33(1): 49-54 (2003)), and cancer therapeutic outcomes (Shipp, et al., Nat Med. 8(1): 68-74 (2002)). Some of these differential expressions are regulated by imprinted or somatic methylation.

The methylation targets are grouped into functionally relevant sets that are useful for focused research (e.g. based on association with a particular pathway or disease; or expressed in particular tissues of interest; or representing a particular genomic region), as well as for more global studies. For example, genes can be grouped according to their biochemical properties, such as, oncogenes/tumor suppressor genes, kinases, phosphatases, and cell surface receptors. Genes can be also grouped based on their involvement in different biological pathways/functions, for example, tumor antigen, signal transduction, apoptosis, angiogenesis, cell cycle control, cell differentiation, DNA repair, cancer metastasis/invasion, drug resistance and detoxification, and transcriptional regulation, etc. The assays are then optimized to achieve a high degree of reliability and specificity within each set.

Selection of CpG Sites to be Interrogated

Several CpG sites within each 5′-regulatory region, including CpGs over the transcriptional start site is targeted, since redundant information from multiple CpG sites can provide a better measurement of the overall methylation status in the interrogated gene. While there are many CpG sites within each CpG island, only those for which robust assays can be designed is used. Each potential CpG site is BLAST searched against human dbSNP databases to avoid any potential “polymorphic” CpG site (i.e. the “methylation-resistant” site), to ensure clean data interpretation. If desired, the consequence of the polymorphic CpG sites, for example, their effect on methylation of adjacent CpG sites, and subsequently on gene expression level can be determined.

It has been estimated that the human genome has 26,000 to 45,000 CpG islands (Antequera, et al., Proc Natl Acad Sci U S A. 90(24): 11995-9 (1993), Ewing, et al., Nat Genet. 25(2): 232-4 (2000)). Among them, those found in gene 5′-regulatory regions are the most biologically significant ones. The work does not only utilize CpG rich promoters, but also investigates less CpG rich promoters since they might also be subjected to aberrant methylation and silencing.

In order to correctly identify genes and their upstream regulatory regions, public databases such as NCBI RefSeq, UCSC Human Genome Project (HGP) Working Draft, ENSEMBL and Unigene are utilized. These databases annotate known or verified genes according to sequence similarity to mRNA, EST and protein sequences, and novel genes predicted using gene prediction programs. They also contain reference links to genetic and physical mapping data, as well as other features such as CpG islands. The accuracy of these annotated features is confirmed by re-running gene prediction programs and searching for sequence similarity to mRNA, EST, and known protein sequences. Existing promoter databases such as Eukaryotic Promoter Database (EPD) (Praz, V., et al., Nucleic Acids Res. 30(1): 322-4 (2002)), are used to identify the 5′-regulatory sequences. Meantime, promoter and first exon prediction algorithms such as FirstEF (Davuluri, et al., Nat Genet. 29(4): 412-7 (2001)) are used to identify potential regulatory sequences. This approach provides a well-annotated collection of regulatory sequences of human genes and allow for identification of the CpG sites within these regulatory regions and design assay probes as described above.

A CpG site within a CpG island doesn't automatically qualify it as a biologically significant methylation target and art knowledge is applied in order to design most valuable methylation assays. In the past few years, tremendous progress has been made in the epigenetics field, which uncovered many epigenetic regulation mechanisms in various biological pathways (Strichman-Almashanu, et al., Genome Res. 12(4): 543-54 (2002)), and cancers (Widschwendter, et al., Oncogene 21(35): 5462-82 (2002), Tsou, et al., 21(35): p. 5450-61 (2002)). Moreover, large methylation projects such as bisulfite sequencing of CpG island-enriched libraries (Cross, et al., Nat Genet. 6(3): 236-44 (1994)), or entire human chromosomes or even the entire genome are likely to be developed. These efforts should produce tremendous amounts of methylation data and reveal hundreds of thousands of new methylation sites in many different tissue types.

Assay Development, and Array Data Extraction and Analysis

Once the assays are designed, they are tested with publicly available genomic DNAs isolated from various cancerous or normal human tissues or cell lines of different tissue origins, and obtain tissue-specific methylation profiles for individual genes (CpG sites). These methylation profiles serve as references for analyzing unknown samples. Building on SNP genotyping technology, a quantitative metric to guide the methylation assay development is formulated and provide a quality assurance to data generated in a production setting. The metric takes into consideration all aspects of assay performance and data quality (e.g. assay specificity and quantitation), including efficiency of bisulfite conversion, overall signal intensity of all targeted CpG sites, concordance among the measurements of the three CpG sites within each gene, specificity of detection in control samples (e.g. plasmids, reference samples as well mixtures of the reference samples), and measurement variations in replicated samples, etc.

In addition the quantitative performance of methylation detection is tested at various multiplexing levels, for example, high (>1000-plex), medium (300-plex), and low (<100-plex), and validate the specificity and sensitivity of the assays at high multiplexing levels. Meanwhile, as a measurement of the assay specificity, concordance of methylation profiles generated from a given sample at different multiplexing levels are compared. Finally, methylation-specific PCR is used to validate some of the array results (Herman, et al., Proc Natl Acad Sci U S A. 93(18): 9821-6 (1996)). All qualified assays are re-pooled and used for large-scale DNA methylation profiling.

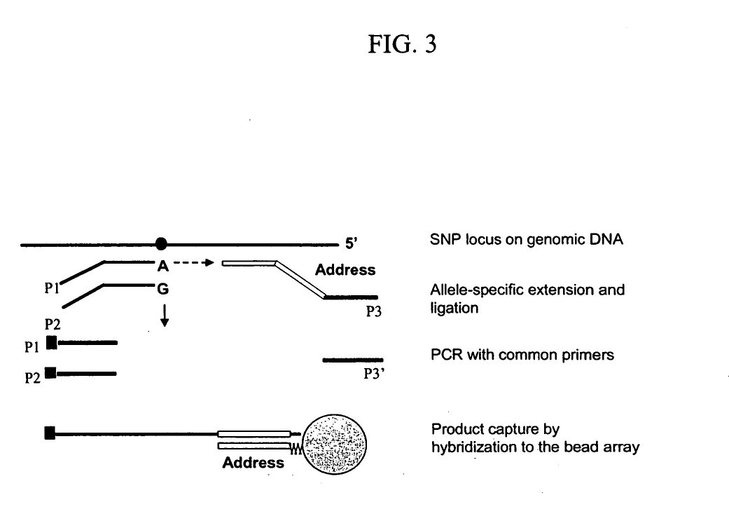

Since the sequence complexity is significantly reduced following bisulfite conversion of the genomic DNA, the assay oligo/genomic DNA annealing protocol is optimized to minimize cross-hybridization. Tetramethylammonium (TMACl) (De Murcia, et al., Biophys Chem. 8(4): 377-83 (1978), Sorg, et al., Nucleic Acids Res. 19(17): 4782 (1991)), and/or Betaine (Rees, et al., Biochemistry, 32(1): 137-44 (1993)) is used to normalize the base composition dependence of DNA/oligo hybridization. Nevertheless, high locus specificity should be achieved by the requirement that both ASO and LSO oligos need to hybridize to the same genomic target site and then get extended and ligated (FIG. 3).

The existing SNP genotyping software is modified and adapted for the methylation data analysis. The current software takes the raw intensity data and transforms them into a genotype call using a clustering algorithm (FIG. 10). However, for most of the methylation data analysis cluster analysis cannot be utilized due to the need not only to distinguish three methylation states of each locus (unmethylated, methylated and semi-methylated), but do it in a more quantitative manner, for example, estimate percentage of methylation of certain loci in a given sample. The methylated and unmethylated reference samples and assay controls are used in every experiment for software calibration. Assay intensity data of unknown sample is compared to those obtained with the reference samples, and used to calculate the methylation level of the locus of interest. Software is developed for comparison of various samples and detection of differential methylation profiles to allow for identification of differences between normal tissues and tumors, and/or create tissue-specific methylation profiles for genes and loci of interest.

Search for Specific Methylation Patterns in Different Cancer Types or Cancer Stages

Once the gene-specific methylation assays are developed as described above, a large-scale DNA methylation survey is carried out in a large number of samples. The experiment is designed to compare methylation patterns in (1) normal and cancerous tissues; (2) different cancer types or cancer stages; (3) or responsive to (or associated with) treatment with certain growth factors or drugs, activation of oncogenes or inactivation of tumor suppressor genes, changes in a developmental program, etc. The main objective is to find unique methylation patterns for specific cancer types/stages and develop molecular markers for classification and diagnosis of cancers, which can be used to complement existing morphological and clinical parameters. This can be particularly useful for cancer types which appear similar by histological assessments, but follow different clinical courses (e.g. different therapeutic responses). The results also provide important clues to the mechanisms of specific cellular responses; and this information can prove critical for devising strategies for cancer prevention and treatment.

Malignant tissues obtained by laser capture microdissection (LCM) are used to identify specific cell and tissue types for methylation profiling. In these cases, a more sensitive strategy is employed, which involves DNA amplification after bisulfite conversion. If an assay can be established to detect tumor-specific methylation patterns in a very small amount of diseased tissue in the presence of a large amount of normal tissue, it may find wide application in clinical cancer diagnosis.

As a pilot study, DNA samples isolated from 98 lung tissues and 101 breast tissues, including both normal and cancerous tissues, are used. Among these tissues, 169 (98 lung and 71 breast) are frozen and 30 (breast) are paraffin-fixed. These tumor tissues are classified upon resection, and basic (anonymous) data about each tumor is kept in the tumor bank database. The tissues were resected, sent to the Pathology Department for pathological examination, and then sent to the tumor bank with the initial pathology report. The tissue was quickly frozen and stored at −80° C. The tissue procurement, storage, and documentation of clinical specimens are very well documented, in accordance with guidelines for human subject research. Thirty of the 101 breast tumor tissues are formalin-fixed, paraffin-embedded tissues. Slides were made from the fixed tissue block and stored at room temperature. Each tumor sample was examined by a pathologist to confirm the clinical diagnosis. Basic (anonymous) data about each tumor, such as the information on the gender, age, and ethnic background of the patient as well as diagnosis are available to us for data analysis.

Initially, DNA methylation profiles are generated for these samples for a list of 146 genes, selected fully based on their biological functions (see Appendix I). If both DNA and RNA samples are available for a subject in the study, both DNA methylation and gene expression is measured, including allele-specific expression, using a sensitive RNA profiling method (Fan, et al., Genome Res. Submitted (2003), Yeakley, et al., Nat Biotechnol. 20(4): 353-8 (2002)). Gene-specific as well as allele-specific probes are designed to measure expression levels of specific transcripts and their isoforms. Cross-referencing gene expression results to DNA methylation data confirms not only the gene silencing caused by DNA methylation, but also helps interpret the association study results. Once specific methylation patterns are derived from this preliminary study, they are validated in (larger) independent sample sets.

Finally, comparing results obtained from human and animal studies sheds light on the underlying molecular mechanisms of tumorigenesis. For example, there is a well-characterized rat mammary tumor model involving mammary glands from virgin and parlous animals exposed to MNU (Sivaraman, et al., Carcinogenesis 19(9): 1573-81 (1998)). It provides an excellent biological model for studying molecular events in human breast cancer, especially those occurred in early cancer developments leading to mammary tumorigenesis (Russo, et al., Br J Cancer 64(3): 481-4 (1991)).

Bioinformatics, Array Information Management, and Statistical Data Analysis

Software development focuses on algorithms and software tools to process and analyze the large amount of methylation assay data as efficiently as possible. A database developer/administrator organizes, track and maintain all the methylation site information, primer design, sample information, the day-to-day experimental data, as well as design and implement web browser interfaces to provide search, query and report functions.

To develop a robust and high-throughput technology for simultaneous measurement of methylation at many specific sites in many samples, a highly efficient analysis and data export process is needed. Algorithms for analyzing data to determine methylation status automatically are developed. Again, this is based on experience with analysis of SNP genotyping data. There are some important differences in how the data is analyzed, such as the requirement for more quantitative analysis and output as described above. Experiments is designed and analysis procedures developed to determine the quantitative limits of our system, such as the limit of detection of methylated DNA in a mixed sample, and linearity of signal as a function of amount of methylated DNA.

Once tools are developed to extract information about methylation status from raw data, and to determine the significance of the measurements and assign confidence indices, the focus is on analyses of patterns of methylation and their correlations with different phenotypes. For example, analyses are carried out to detect and verify any correlations between specific methylation patterns and particular cancer types. Techniques (methods) to perform this type of analysis are the subject of intensive research in the microarray field. Many powerful algorithms/tools have been developed, such as supervised or unsupervised hierarchical clustering analysis (Dhanasekaran, et al., Nature 412(6849): 822-6 (2001), Eisen, et al., Proc Natl Acad Sci U S A. 1998. 95(25): 14863-8 (1991), Khan, et al., Nat Med. 7(6): 673-9 (2001)), K-means clustering, bootstrapping or jackknife (Kerr, M. K. et al. Proc Natl Acad Sci U S A. 98(16): 8961-5 (2001)), principal component analysis, etc. However, each of these methods has its own advantages and limitations; no clear advantage exists for any given algorithm in application to our study. Therefore, multiple algorithms are tested and the most suitable ones are selected to carry out the analyses.

The technology is upscaled to meet commercial requirements by implementing the entire process, including sample preparation, bisulfite treatment, genotyping-based assay and PCR amplification on a robotic platform; increasing the level of multiplexing to at least 96-plex, and as high as 1,500-plex; and reducing the amount of genomic DNA required such that, on average, <1 ng of genomic DNA is required per methylation site analyzed.

The assay described herein allows measurement of the methylation status in at least 1,000 human genes' 5′-regulatory regions, and validate the sensitivity and specificity of the assays at high multiplexing levels. The assay further allows for a systematic search for specific methylation patterns in different cancer types and cancer stages.

Overall, this Example describes a system for methylation detection by leveraging various technologies for high-throughput array-based assays and SNP genotyping, and to validate the technology in real-world applications. The technology is highly scalable, both in terms of the number of assays carried out on a single sample, and the number of samples that can be processed in parallel. Furthermore, it can be used has the potential for broad application in many areas of cancer and fundamental biomedical research. The assays and assay protocols, and the specific methylation patterns (in various cancers) to be developed in this study can generally be useful to the research community.

Throughout this application various publications have been referenced. The disclosures of these publications in their entireties are hereby incorporated by reference in this application in order to more fully describe the state of the art to which this invention pertains.

The term “comprising” is intended herein to be open-ended, including not only the cited elements, but further encompassing any additional elements.

Unless defined otherwise, all technical and scientific terms used herein have the same meaning as commonly understood by one of ordinary skill in the art to which the invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

TABLE 1
Cancer-associated genomic markers and corresponding target nucleic acid probes.

| | GENBANK | | | | |
|---|---|---|---|---|---|
| | ACCESSION | | | | |
| TARGET CPG SITE | # | PROBE SEQUENCE (ASO1) | PROBE SEQUENCE (LSO1) | PROBE SEQUENCE (ASO2) | PROBE SEQUENCE (LSO2) |
| GI29736559_ESR2_1R | NT_026437 | GCCCTCACACTTCTACACCG | GTCCCCAAAACTAAAAAACATCC | AACACCCTCACACTTCTACACCA | ATCCCCAAAACTAAAAAACATCC |
| (SEQ ID 1)) | | | | | |
| GI29736559_ESR2_2R | NT_026437 | CCGACTTTATCACACACCTACG | CGCCAAACTAAAATCGAACC | CTTCCAACTTTATCACACACCTACA | CACCAAACTAAAATCAAACCCCT |
| (SEQ ID 2) | | | | | |
| GI29736559_ESR2_3R | NT_026437 | CGACTTCCAAACAATAATAAACG | ATCCCTACGCGAAAACGTAAC | CCCAACTTCCAAACAATAATAAACA | ATCCCTACACAAAAACATAACAAACA |
| (SEQ ID 3) | | | | | |
| GI29791372_TGFBR2_1R | NT_022517 | CCCTAATAAATCAAAACATCTACCG | CTCCCTCAACTTTCTTCAAATT | TCCCTAATAAATCAAAACATCTACCA | CTCCCCTCAACTTTCTTCAAATT |
| (SEQ ID 4) | | | | | |
| GI29791372_TGFBR2_2R | NT_022517 | ACTTTCAACTACCCCTCACCG | CCTCCCACACCACTCAAAAATT | AACTTTCAACTACCCCTCACCA | CCTCCCACACCACTCAAAAATT |
| (SEQ ID 5) | | | | | |
| GI29791372_TGFBR2_3R | NT_022517 | GTCCAACCCCTAACTCTCTCG | AACTACCAATCATATTTCCTAAACCA | ACATCCAACCCCTAACTCTCTCA | AACTACCAATCATATTTCCTAAACCA |
| (SEQ ID 6) | | | | | |
| GI29791384_TP73_1R | NL_004321 | ACCCGAATCTCTCCTAACCG | CGCCACTAACGCTAAACTCCT | TAACACCCAAATCTCTCCTAACCA | CACCACTAACACTAAACTCCTCAA |
| (SEQ ID 7) | | | | | |
| GI29791384_TP73_2R | NT_004321 | ACTCTATACCCGACGCCTACG | TTCCCCCGAACTCCCTACTAT | CCAACTCTATACCCAACACCTACA | TTCCCCCAAACTCCCTACTATC |
| (SEQ ID 8) | | | | | |
| GI29791384_TP73_3R | NT_004321 | CTAACGCCGAACCTACTTCG | CCCTACGTAAACGACCTCGC | AACCCTAACACCAAACCTACTTCA | CCCTACATAAACAACCTCACCAA |
| (SEQ ID 9) | | | | | |
| GI29791621_PTGS2_1R | NT_004487 | CGCCAAATATCTTTTCTTCTTCG | AATCTTTACCCGAACGCTTCC | CCACCAAATATCTTTTCTTCTTCA | AATCTTTACCCAAACACTTCCAA |
| (SEQ ID 10) | | | | | |
| GI29791621_PTGS2_3R | NT_004487 | TATCCCGACGTAACTTCCTCG | CCCTCTAAAAACGTACAAACCAA | AATCTATCCCAACATAACTTCCTCA | CCCTCTAAAAACATACAAACCAAA |
| (SEQ ID 11) | | | | | |
| GI29793179_DAPK1_1R | NT_023935 | CCGACCTAACAAAACAACTCG | AAATAAATAAACCGCGCCG | ACACCAACCTAACAAAACAACTCA | AAATAAATAAACCACACCACCAAC |
| (SEQ ID 12) | | | | | |
| GI29793179_DAPK1_3R | NT_023935 | AACCTTCTTACCTTCAAACCTCG | CTCCAACACCAATCCGACA | AAACCTTCTTACCTTCAAACCTCA | CTCCAACACCAATCCAACAAA |
| (SEQ ID 13) | | | | | |
| GI29794150_CASP8_1R | NT_005403 | AAATACTCCCATTCTCCACCG | TTATCACACACTTATTTCTCACTTCC | AAAAATACTCCCATTCTCCACCA | TTATCACACACTTATTTCTCACTTCC |
| (SEQ ID 14) | | | | | |
| GI29794150_CASP8_2R | NT_005403 | ACTACCACTCCCCAAAAAACG | CCTCCTCGTCTCCTCCC | CACTACCACTCCCCAAAAAACA | CCTCCTCATCTCCTCCCAC |
| (SEQ ID 15) | | | | | |
| GI29794150_STAT1_1R | NT_005403 | CGACGAAAATACCCCAACG | GTAAACGAAACAACGACCCG | AAACCAACAAAAATACCCCAACA | ATAAACAAAACAACAACCCACAAA |
| (SEQ ID 16) | | | | | |
| GI29794150_STAT1_2R | NT_005403 | GCGCTCAACCAATTAAACG | GACTATTCCGTAAACGCCACC | ACTCCACACTCAACCAATTAAACA | AACTATTCCATAAACACCACCACC |
| (SEQ ID 17) | | | | | |
| GI29794150_STAT1_3R | NT_005403 | AATTCCCAACGTAACAACACG | AACTAAACTACAACTCACCCAACC | CCAATTCCCAACATAACAACACA | AACTAAACTACAACTCACCCAACCA |
| (SEQ ID 18) | | | | | |
| GI29794559_CDKN2A_1R | NT_008413 | ACTCACCCCTCCTTTCTACCG | TCCTTCCTTTCCTTACCCTACTTT | AACTCACCCCTCCTTTCTACCA | TCCTTCCTTTCCTTACCCTACTTT |
| (SEQ ID 19) | | | | | |
| GI29794559_CDKN2A_2R | NT_008413 | GCGTCCGAATTCCTAAACG | AAACCGAACCTCGCTTAAACC | ACCCACATCCAAATTCCTAAACA | AAACCAAACCTCACTTAAACCAC |
| (SEQ ID 20) | | | | | |
| GI29794559_CDKN2A_3R | NT_008413 | AACCCGCGAAATTTAAAACG | ATCCAAACAAACCGCAAACT | TCCAACCCACAAAATTTAAAACA | ATCCAAACAAACCACAAACTCC |
| (SEQ ID 21) | | | | | |
| GI29794559_CDKN2B_1R | NT_008413 | CAATACAACCAACATTCCTAACG | CTCCCTAACCCAATCTCTAACG | AACAATACAACCAACATTCCTAACA | CTCCCTAACCCAATCTCTAACACA |
| (SEQ ID 22) | | | | | |
| GI29794559_CDKN2B_2R | NT_008413 | CAACTCCTAATCCCCAATAAACG | AACAAAAAATTTCTATTCCTTCAACC | CAACTCCTAATCCCCAATAAACA | AACAAAAAATTTCTATTCCTTCAACC |
| (SED ID 23) | | | | | |
| GI29794559_CDKN2B_3R | NT_008413 | CATCTTTAAACAAACTTCCCCG | CCTCGTAACGCGTCGAC | AACATCTTTAAACAAACTTCCCCA | CCTCATAACACATCAACCCAAAC |
| (SEQ ID 24) | | | | | |
| GI29796774_MYC_1R | NT_008046 | AAACAAACAACCTCCCTCTCG | CCTAACCCAACTCTAAAACAAACAA | AAAACAAACAACCTCCCTCTCA | CCTAACCCAACTCTAAAACAAACAA |
| (SEQ ID 25) | | | | | |
| GI29796774_MYC_2R | NT_008046 | CTTTAATTTCTCCCAAACCCG | CAACCCGAAACTATTACAAACCG | ACCTTTAATTTCTCCCAAACCCA | CAACCCAAAACTATTACAAACCAA |
| (SEQ ID 26) | | | | | |
| GI29796774_MYC_3R | NT_008046 | AAAAATCTCTACTAACTCCCCCG | CTCGATCCACAAACTCTCCACT | CAAAAATCTCTACTAACTCCCCCA | CTCAATCCACAAACTCTCCACTT |
| (SEQ ID 27) | | | | | |
| GI29797939_APC_1R | NT_034772 | AACAACACCTCTCACGCATACG | ATTATAATCTTCCCACCTCCCAC | CAACAACACCTCTCACACATACA | ATTATAATCTTCCCACCTCCCAC |
| (SEQ ID 28) | | | | | |
| GI29797939_APC_2R | NT_034772 | CAAAACAACGAAACAATACCCG | CAAACGAAACGCAACACC | AACAAAACAACAAAACAATACCCA | CAAACAAAACACAACACCCATTA |
| (SEQ ID 29) | | | | | |
| GI29797939_APC_3R | NT_034772 | CGAAAACCTAACCGCTACTCG | AAAAAACCTACGAACTCAAACCC | CACCAAAAACCTAACCACTACTCA | AAAAAACCTACAAACTCAAACCCA |
| (SEQ ID 30) | | | | | |
| GI29798364_HIC1_1R | NT_010718 | CCCCTTCCCTCAACTAAACG | AAACATCAACCCTACGACCTAAA | CTTCCCCTTCCCTCAACTAAACA | AAACATCAACCCTACAACCTAAACA |
| (SEQ ID 31) | | | | | |
| GI29798364_HIC1_2R | NT_010718 | CTCCTACTCCTTCTCCTAATCCG | ACGAACCGACCTAAACTCCC | CTCCTACTCCTTCTCCTAATCCA | ACAAACCAACCTAAACTCCCACT |
| (SEQ ID 32) | | | | | |
| GI29798364_HIC1_3R | NT_010718 | TAAAACAACTTCTCCCGAAACG | AAAACGCTAATTCCTCGACTCC | AACTAAAACAACTTCTCCCAAAACA | AAAACACTAATTCCTCAACTCCC |
| (SEQ ID 33) | | | | | |
| GI29798364_TP53_1R | NT_010718 | TATCTACGACACCAAATCGACG | AAATCCTAACTCTACACCCTCCTC | ATTAATATCTACAACACCAAATCAACA | AAATCCTAACTCTACACCCTCCTC |
| (SEQ ID 34) | | | | | |
| GI29798364_TP53_2R | NT_010718 | CCATTTCCTTTACTTCCTCCG | CAAACGAATTACTTACCCTTACTTATCA | CTCCATTTCCTTTACTTCCTCCA | CAAACAAATTACTTACCCTTACTTATCA |
| (SEQ ID 35) | | | | | |
| GI29798364_TP53_3R | NT_010718 | CGCCGCCTACAAAAAACG | AACAAATCTTACACCTCTTCTACATCTC | CCCCACCACCTACAAAAAACA | AACAAATCTTACACCTCTTCTACATCTC |
| (SEQ ID 36) | | | | | |
| GI29798595_BRCA1_1R | NT_010755 | CACGAAAACCAAAAAACTACCG | TAAACAACAACCTCTCAAAATACG | CCACAAAAACCAAAAAACTACCA | TAAACAACAACCTCTCAAAATACAAAA |
| (SEQ ID 37) | | | | | |
| GI29798595_BRCA1_2R | NT_010755 | AATAACCAATCCAAAACCCCG | AAAACGCTTAACTCTTTCTATCCC | TAAATAACCAATCCAAAACCCCA | AAAACACTTAACTCTTTCTATCCCTCC |
| (SEQ ID 38) | | | | | |
| GI29798595_BRCA1_3R | NT_010755 | CAAATAAAATTCTTCCTCTTCCG | CTCTTTCCTTTTACGTCATCCG | CTCAAATAAAATTCTTCCTCTTCCA | CTCTTTCCTTTTACATCATCCAAAA |
| (SEQ ID 39) | | | | | |
| GI29800185_PTEN_1R | NT_030059 | CTCGAATCAATAACACTACTCAACG | ACCCATCTCAACTTTCATCATCA | AAACTCAAATCAATAACACTACTCAACA | ACCCATCTCAACTTTCATCATCA |
| (SEQ ID 40) | | | | | |
| GI29800185_PTEN_2R | NT_030059 | AAATATTCCCCTTACAAAAACCG | CCCTACATTTCCCTCTACACTAAA | AAAATATTCCCCTTACAAAAACCA | CCCTACATTTCCCTCTACACTAAA |
| (SEQ ID 41) | | | | | |
| GI29800185_PTEN_3R | NT_030059 | CAACCTACTATTATATCGCCAACG | ATATCACCTCATCCGACTCCCTT | AAAACAACCTACTATTATATCACCAACA | ATATCACCTCATCCAACTCCCTT |
| (SEQ ID 42) | | | | | |
| GI29800407_RET_1R | NT_033985 | TACGTAAATACGCGAACCCG | ACTCCTAAATTCCATCCCCG | CAACTCTACATAAATACACAAACCCA | ACTCCTAAATTCCATCCCCAC |
| (SEQ ID 43) | | | | | |
| GI9800407_REL_2R | NT_033985 | CAAACCTACATCCCGCG | AACATCCCTACCCTCTCTATACAA | ATCAACCAAACCTACATCCCACA | AACATCCCTACCCTCTCTATACAACA |
| (SEQ ID 44) | | | | | |
| GI29800407_RET_3R | NT_033985 | CGAAAAACCTAAAAACGAAACG | CCTTCTCTAAATCCGCGAAAT | CCACAAAAAACCTAAAAACAAAACA | CCTTCTCTAAATCCACAAAATCAC |
| (SEQ ID 45) | | | | | |
| GI29800594_TGFB1_1R | NT_011109 | CGTACGCTTCCTAAATAAAACCG | AAACGACTTCAAAACCCCCTAC | CCCATACACTTCCTAAATAAAACCA | AAACAACTTCAAAACCCCCTACC |
| (SEQ ID 46) | | | | | |
| GI29800594_TGFB1_2R | NT_011109 | TCTTTCTCTAATAACCCACACCG | CCGCAAAACCACAACGC | CTCTTTCTCTAATAACCCACACCA | CCACAAAACCACAACACATCTAA |
| (SEQ ID 47) | | | | | |
| GI29800594_TGFB1_3R | NT_011109 | CCAACCTAACTCTCCTTCCG | TCTAAATCCCCCTCCTCTAATCG | CCCAACCTAACTCTCCTTCCA | TCTAAATCCCCCTCCTCTAATCA |
| (SEQ ID 48) | | | | | |
| GI29802188_SNRPN_1R | NT_026446 | TCTTAAATCTTAACAATCCCCG | ACACTACTCCCCGCTTTATTTATT | TCTCTTAAATCTTAACAATCCCCA | ACACTACTCCCCACTTTATTTATTTAA |
| (SEQ ID 49) | | | | | |
| GI29802188_SNRPN_2R | NT_026446 | CCTAAATACCTCCTACGCAAACG | AATTATCCTCCTACGCCGACCT | TCCCTAAATACCTCCTACACAAACA | AATTATCCTCCTACACCAACCTCA |
| (SEQ ID 50) | | | | | |
| GI29802395_GLA_1R | NT_011651 | ATCAATCACGTAAAACGTTCCG | TAACCAATCACCCTCTTCCTTTC | CAATAATCAATCACATAAAACATTCCA | TAACCAATCACCCTCTTCCTTTC |
| (SEQ ID 51) | | | | | |
| GI29802395_GLA_2R | NT_011651 | ACACCAACCTCTAACGATACCG | ATAATTTTCCTCCTTCTTCCCTC | ACACACCAACCTCTAACAATACCA | ATAATTTTCCTCCTTCTTCCCTC |
| (SEQ ID 52) | | | | | |
| GI29802395_GLA_3R | NT_011651 | CTCAAAACCGTAATTTTCAAACG | TTTTCGCCTTACGATCACC | AACTCAAAACCATAATTTTCAAACA | TTTTCACCTTACAATCACCCTTAA |
| (SEQ ID 53) | | | | | |
| GI29802670_AR_1R | NT_011669 | AAAACTAACCTCTCCTACCCTCG | CCACGCTACGCCAACACTTAT | TAAAACTAACCTCTCCTACCCTCA | CCACACTACACCAACACTTATTTCT |
| (SEQ ID 54) | | | | | |
| GI29802670_AR_2R | NT_011669 | CCTAAAACAAATAACACAATACCACG | AACCCGATCTATCCCTATAACGA | TCCTAAAACAAATAACACAATACCACA | AACCCAATCTATCCCTATAACAAA |
| (SEQ ID 55) | | | | | |
| GI29802670_AR_3R | NT_011669 | ATAAAAACGAAACCCGACTCG | AAACTATTACATTTACTCTCCACCTCC | CCAATAAAAACAAAACCCAACTCA | AAACTATTACATTTACTCTCCACCTCC |
| (SEQ ID 56) | | | | | |
| GI29802882_MAGEA1_1R | NT_011726 | CAAATCAATAACGTCACATCCG | ACACCCGAATATTTCCTAACGAA | CACAAATCAATAACATCACATCCA | ACACCCAAATATTTCCTAACAAAA |
| (SEQ ID 57) | | | | | |
| GI29802882_MAGEA1_2R | NT_011726 | CTCAACCCTAATACCCATCCG | CCAACCATTCCACCCTCA | CTCAACCCTAATACCCATCCA | CCAACCATTCCACCCTCA |
| (SEQ ID 58) | | | | | |
| GI29802882_MAGEA1_3R | NT_011726 | CCACTCCAATACTCACTCCCG | AACCCAACCCCCTCTTCATTAT | CCACTCCAATACTCACTCCCA | AACCCAACCCCCTCTTCATTAT |
| (SEQ ID 59) | | | | | |
| GI29804485_CTAG2_1R | NT_025965 | CGAACAAAACAAACAAAATCCG | AACGATAACCGCACAATCC | AACAAACAAAACAAACAAAATCCA | AACAATAACCACACAATCCCAAC |
| (SEQ ID 60) | | | | | |
| GI29804485_CTAG2_2R | NT_025965 | CTTAACTAACGCGAAACCACG | CCACCAAACATACAATTCCAACT | AAAACCTTAACTAACACAAAACCACA | CCACCAAACATACAATTCCAACT |
| (SEQ ID 61) | | | | | |
| GI29804485_CTAG2_3R | NT_025965 | GAATCTCACTCCTAAATTCAACG | ATTCTATCCCTATCCTAATCGCC | CCAAATCTCACTCCTAAATTCAACA | ATTCTATCCCTATCCTAATCACCC |
| (SEQ ID 62) | | | | | |
| GI29804485_G6PD_1R | NT_025965 | GCGAACCTACAAAACCTAACG | ACTCAAACTTCTCTCCGAAACG | TCCACAAACCTACAAAACCTAACA | ACTCAAACTTCTCTCCAAAACAAAA |
| (SEQ ID 63) | | | | | |
| GI29804485_G6PD_2R | NT_025965 | ACTCGATAATAATAAACACGCCG | CCACTTTACAAAACGTCACCG | CAAAAACTCAATAATAATAAACACACCA | CCACTTTACAAAACATCACCACC |
| (SEQ ID 64) | | | | | |
| GI29804485_G6PD_3R | NT_025965 | CCAATAAAACCGACTTAACCCG | GAACAAACGAAAATTCCCGA | CCCAATAAAACCAACTTAACCCA | AAACAAACAAAAATTCCCAAAAA |
| (SEQ ID 65) | | | | | |
| GI29804485_SLC6A8_1R | NT_025965 | CTTCCTTAAACTCTCTCCACCG | TACCCACTAAAAACTACAACTTCCC | CCTTCCTTAAACTCTCTCCACCA | TACCCACTAAAAACTACAACTTCCC |
| (SEQ ID 66) | | | | | |
| GI29804485_SLC6A8_2R | NT_025965 | ATAACAACCATCCTACCTCCCG | TAAAACGACGTCTCCTCCCC | AATAACAACCATCCTACCTCCCA | TAAAACAACATCTCCTCCCCAA |
| (SEQ ID 67) | | | | | |
| GI29804485_SLC6A8_3R | NT_025965 | GACCGACCGTAAAAATAAAACG | TAATAACATCACCCCGAAATCG | CCAACCAACCATAAAAATAAAACA | TAATAACATCACCCCAAAATCAA |
| GI29805823_GSTP1_2R | NT_033903 | CCTCTCCCCTACCCTATAAAACG | ATATACAAACTCCGAAATCGCAA | CCTCTCCCCTACCCTATAAAACA | ATATACAAACTCCAAAATCACAACAA |
| GI29805823_GSTP1_3R | NT_033903 | AACTACGCGACGACTCCG | AAACTCCAAAACGCCCCTCTAC | CAACCAACTACACAACAACTCCA | AAACTCCAAAACACCCCTCTACA |
| GI29807292_BCR_1R | NT_011520 | GAAAAACACCTTCCATAAAAACG | AAATAAATTCCTCACACCTCACAAC | CAAAAAACACCTTCCATAAAAACA | AAATAAATTCCTCACACCTCACAAC |
| GI29807292_BCR_2R | NT_011520 | GCAATACCATAAACGTCACCG | CACTACCCAATCGTCGTATCAAA | ACCACAATACCATAAACATCACCA | CACTACCCAATCATCATATCAAAA |
| GI29807292_BCR_3R | NT_011520 | ACGAATTACACAATCCAATTCG | TATTATTAAAACCTCAATTTCCCAAA | AAACTACAAATTACACAATCCAATTCA | TATTATTAAAACCTCAATTTCCCAAA |
| GI29807292_TIMP3_1R | NT_011520 | AATTAATAACCAACCAAAAACTCG | CACCCCTTTCCACTACTCCTT | AATAATTAATAACCAACCAAAAACTCA | CACCCCTTTCCACTACTCCTT |
| GI29807292_TIMP3_2R | NT_011520 | TCTCTTTCCCTTTCTCTTCCG | TTCATCCTATTAAAAATACCACAAATT | TCTCTTTCCCTTTCTCTTCCA | TTCATCCTATTAAAAATACCACAAATT |
| GI29807454_CALCA_1R | NT_009237 | AATAAATTTCACCTCTCAATCCG | CCTAATTTTAAACCCTCGCCC | CATAATAAATTTCACCTCTCAATCCA | CCTAATTTTAAACCCTCACCCC |
| GI29807454_CALCA_2R | NT_009237 | AAAATCCTTTACCCCTAAATTACG | CACCCTCATACTTCCAAAACCTA | AAAAATCCTTTACCCCTAAATTACA | CACCCTCATACTTCCAAAACCTA |
| GI29807454_KAI1_1R | NT_009237 | GCACCAACCTAAACTCAACG | CTTCCTAATCCTACCCTACCCTA | CCACACCAACCTAAACTCAACA | CTTCCTAATCCTACCCTACCCTA |
| GI29807454_KAI1_2R | NT_009237 | CCCAACTAAAACTAAAACCCCG | CCCGCTACGAAATCTCTCCT | ACCCAACTAAAACTAAAACCCCA | CCCACTACAAAATCTCTCCTCTATC |
| GI29807454_KAI1_3R | NT_009237 | GCCTCCTAATAAAAACCCCG | CTTAAAACACAAACCGCTCCC | ACCACCTCCTAATAAAAACCCCA | CTTAAAACACAAACCACTCCCAC |
| GI29807454_MYOD1_1R | NT_009237 | TACCTCTCTCCAAATCTCTCACG | CCTAATTTCTACAACCGCTCTACC | TTACCTCTCTCCAAATCTCTCACA | CCTAATTTCTACAACCACTCTACCC |
| GI29807454_MYOD1_2R | NT_009237 | TACGTATCTCTCAACCTCTTTCG | TCCCTCTTTCACGATCTCACTC | CTTTCTACATATCTCTCAACCTCTTTCA | TCCCTCTTTCACAATCTCACTCC |
| GI29807454_MYOD1_3R | NT_009237 | AATATCAAAACCTCTACGACCCG | TTATCTCGAACTCGCCCACTT | AAAATATCAAAACCTCTACAACCCA | TTATCTCAAACTCACCCACTTCAA |
| GI29808625_CDH1_2R | NT_010498 | GAACCCAATAAAATCAAAACCG | ACAAATCCCATAACCCACCTAAA | CAAACCCAATAAAATCAAAACCA | ACAAATCCCATAACCCACCTAAA |
| GI29808625_CDH1_3R | NT_010498 | CCTCAACCAATCAACGATACG | AAAACGATACCTCCGAAACTCAC | ACCCTCAACCAATCAACAATACA | AAAACAATACCTCCAAAACTCACC |
| GI29823164_TYMS_1R | NT_010859 | TTAAAAACCGTCTAATCGACCG | GCCTTCTCTAAACCAACAACACA | CCTTTAAAAACCATCTAATCAACCA | ACCTTCTCTAAACCAACAACACAA |
| GI29823164_TYMS_2R | NT_010859 | TCCGTTCTATACCACACCCG | AACTCCTACGTTTCCCCCTAAC | AACTCCATTCTATACCACACCCA | AACTCCTACATTTCCCCCTAACA |
| GI29823171_BCL2_1R | NT_025028 | TACTTCATTCTCTACACAACCCG | CCGATTTCCTATACGTAACGTCA | CTTACTTCATTCTCTACACAACCCA | CCAATTTCCTATACATAACATCACACA |
| GI29823171_BCL2_2R | NT_025028 | CCTCCTAATCCTACGCGACG | CGCTAACTACGACCGCCTC | CCTCCTCCTAATCCTACACAACA | CACTAACTACAACCACCTCCCA |
| GI29823171_BCL2_3R | NT_025028 | AACGCTCGAAACGAACTACG | GACGAAAACTCCGAAAAACGAC | CCATCAACACTCAAAACAAACTACA | AACAAAAACTCCAAAAAACAACC |
| GI29734309_SYK_1R | NT_008476 | CCAACTCCGAACTCATAAACG | GATCAACAAAACGAACCAAAACG | ACCCAACTCCAAACTCATAAACA | AATCAACAAAACAAACCAAAACAA |
| GI29734309_SYK_2R | NT_008476 | ACTCCTCTACACCTACCGCCG | CTAAACCGATTCCGCGAAC | AAACTCCTCTACACCTACCACCA | CTAAACCAATTCCACAAACCTCA |
| GI29734309_SYK_3R | NT_008476 | ACCGATACGATCCATATCCCG | ACAACCCCACCTTCTCTACCTAC | AAACCAATACAATCCATATCCCA | ACAACCCCACCTTCTCTACCTAC |
| GI29740881_HTR1B_1R | NT_007299 | AAACCAAAACTTACAAACCAACG | AAAATCCCGAAATCTTACATTCC | AAAACCAAAACTTACAAACCAACA | AAAATCCCAAAATCTTACATTCCC |
| GI29740881_HTR1B_2R | NT_007299 | TTTATCCCCAATTAATAATTCCG | AAATTCCTCAATTATTCCTCCGC | AATTTATCCCCAATTAATAATTCCA | AAATTCCTCAATTATTCCTCCACC |
| GI29740881_HTR1B_3R | NT_007299 | TTTATAACTCCGTCTCCGCG | AACAACTCGTCCGAATAACCAA | CCCTTTTATAACTCCATCTCCACA | AACAACTCATCCAAATAACCAAA |
| GI29741420_MGMT_1R | NT_008818 | CTACCCTATACGCCTAACCCG | ATAACCCTTCGACCGATACAAAC | CCACTACCCTATACACCTAACCCA | ATAACCCTTCAACCAATACAAACC |
| GI29741420_MGMT_2R | NT_008818 | ACGTACCGACGTCCAACG | AAATACGCAAACTACCTCAAACC | AACCAACATACCAACATCCAACA | AAATACACAAACTACCTCAAACCCA |
| GI29741420_MGMT_3R | NT_008818 | CACTCGAACACGTAACAAATCG | TTACACGCCCGCGAACTAT | AACCACTCAAACACATAACAAATCA | TTACACACCCACAAACTATCCCT |
| GI29791697_CDC25A_1R | NT_005825 | CTTTCCTAATTAACGCCAAACG | AATCCACCAATCAATAAACAACTTC | CCCTTTCCTAATTAACACCAAACA | AATCCACCAATCAATAAACAACTTC |
| GI29791697_CDC25A_2R | NT_005825 | AACCGAACCAAAACTAAATCCG | CCAAACCTCCACAAATCTTCCTT | AAAACCAAACCAAAACTAAATCCA | CCAAACCTCCACAAATCTTCCTT |
| GI29791697_CDC25A_3R | NT_005825 | AATAACCCCCAAAACTAAACTCG | CCACCAAAACCCAACTCTAAAA | CAATAACCCCCAAAACTAAACTCA | CCACCAAAACCCAACTCTAAAA |
| GI29791697_PTHR1_1R | NT_005825 | CAAAAACAAATCCAAATATACCG | TATTCAAATACCCAAAACTAAACCC | AACAAAAACAAATCCAAATATACCA | TATTCAAATACCCAAAACTAAACCC |
| GI29791697_PTHR1_2R | NT_005825 | CAACCCTAAACATCTAAACACCG | CACACTTAAATCTACCTCTATTACCTCC | CAACCCTAAACATCTAAACACCA | CACACTTAAATCTACCTCTATTACCTCC |
| GI29791697_PTHR1_3R | NT_005825 | TCATTCCACTCCAACCCG | ATCCTAACCCCTACAACCCC | TCATTCCACTCCAACCCA | ATCCTAACCCCTACAACCCC |
| GI29792366_SOD3_1R | NT_006316 | CTAAAAAATCTCCCTCTTATCTCG | AAAATACCTATCCCTAAATAAAAATCATT | TCTAAAAAATCTCCCTCTTATCTCA | AAAATACCTATCCCTAAATAAAAATCATT |
| GI29792366_SOD3_2R | NT_006316 | AACCCTTCCACCCTACCG | CCTTCCTTCTAACCCGCTAAA | CAACCCTTCCACCCTACCA | CCTTCCTTCTAACCCACTAAAAA |
| GI29792366_SOD3_3R | NT_006316 | CACAAATCCTAAAATACCAAAACG | AAACGTACTTTCTTAAACCTTAAACG | CACAAATCCTAAAATACCAAAACA | AAACATACTTTCTTAAACCTTAAACAAAA |
| GI29792503_VHL_1R | NT_005927 | AACTACAATAAACCAAACTCGCG | CACTACACTCCAACCCGAAC | CAAAACTACAATAAACCAAACTCACA | CACTACACTCCAACCCAAACAAC |
| GI29792503_VHL_3R | NT_005927 | GTATAAAATACGCCACCCTCG | ACCTTATTACGACGTCGACACAT | CCCATATAAAATACACCACCCTCA | ACCTTATTACAACATCAACACATTACA |
| GI29793120_CD1A_1R | NT_004668 | CCCATCCCCTAATAATCACG | ATTACCCATTAAATCTTAATTCCCC | TCCCATCCCCTAATAATCACA | ATTACCCATTAAATCTTAATTCCCC |
| GI29793120_CD1A_2R | NT_004668 | AACAATAACAAACATCTCTTATACCG | ACCTTACACCTCAACCCATTT | CATAACAATAACAAACATCTCTTATACCA | ACCTTACACCTCAACCCATTT |
| GI29793120_S100A2_1R | NT_004668 | ACTATAACCCAACCCAACTACG | CTATATATAAAACAACCCCATTTCTCA | AAACTATAACCCAACCCAACTACA | CTATATATAAAACAACCCCATTTCTCA |
| GI29793120_S100A_2R | NT_004668 | AACACTAAACCATAAAATCTCAACG | ATACTCACAACCTCTCACACAAAA | AAACACTAAACCATAAAATCTCAACA | ATACTCACAACCTCTCACACAAAA |
| GI29793234_ABCC5_1R | NT_005962 | CCTACAATCTACGCAAATACCG | CTTTCTCACTTTAACACCAACTACCC | AACCCTACAATCTACACAAATACCA | CTTTCTCACTTTAACACCAACTACCC |
| GI29793234_ABCC5_2R | NT_005962 | CCTATATTCGCACACAATACTATTCG | AAACAAAACAATCCCACCCTAC | CCCTATATTCACACACAATACTATTCA | AAACAAAACAATCCCACCCTACA |
| GI29793234_ABCC5_3R | NT_005962 | ACCGACCTAACGACTCTCAACG | ATTAAACGCGAACTACGATAACC | CTAACCAACCTAACAACTCTCAACA | ATTAAACACAAACTACAATAACCTTCACA |
| GI29793234_HRASLS_2R | NT_005962 | ACACCACACACACACACACG | CAAATAACCAACAAAAACCTCCA | CACACCACACACACACACACA | CAAATAACCAACAAAAACCTCCA |
| GI29793234_HRASLS_3R | NT_005962 | AACCGATTATCTTTATAACCGCG | CGCCTAACACAACGCCTAATAC | CAAAACCAATTATCTTTATAACCACA | CACCTAACACAACACCTAATACCCT |
| GI29793705_CD2_1R | NT_004754 | CACAAAAAACAAAAACGCG | ACAAACCAACCCTTCTAATATACTCC | CACATACACAAAAAACAAAAACACA | ACAAACCAACCCTTCTAATATACTCC |
| GI29793872_RASSF1_1R | NT_006014 | CCTCACCCCAAATAAAAACTCG | AACTTCCTACCCCACCCAATAAA | TCCTCACCCCAAATAAAAACTCA | AACTTCCTACCCCACCCAATAAA |
| GI29793872_RASSF1_2R | NT_006014 | CTACACCCAAATTTCCATTACG | GACTCTCCTCAACTCCTTCCC | CCTACACCCAAATTTCCATTACA | AACTCTCCTCAACTCCTTCCCAC |
| GI29793872_RASSF1_3R | NT_006014 | AATCACGATCCAACCTCTACCG | AACCCCAATCTCCGCAATAAA | AAATCACAATCCAACCTCTACCA | AACCCCAATCTCCACAATAAAA |
| GI29794089_PRDM2_1R | NT_004873 | ATACCCAAAAACAATAACCAACG | TACCCTATACCCGCGATATTCC | AATACCCAAAAACAATAACCAACA | TACCCTATACCCACAATATTCCAA |
| GI29794089_PRDM2_2R | NT_004573 | GAACGACGAAACAATAACCTACG | AACTAAAAAACTCCGAAACCCC | TCAAACAACAAAACAATAACCTACA | AACTAAAAAACTCCAAAACCCC |
| GI29794089_PRDM2_3R | NT_004873 | TCATCAAAACATCTATAAATCGTCG | CTCTACAATCCATTTATTTACCCG | TCTTCATCAAAACATCTATAAATCATCA | CTCTACAATCCATTTATTTACCCAC |
| GI29794150_NCI_1R | NT_005403 | TCCAAAACTACCCAAACCTACG | ACCCAACCACATTAACGAACC | ACTCCAAAACTACCCAAACCTACA | ACCCAACCACATTAACAAACCA |
| GI29794150_NCI_2R | NT_005403 | GCGATCCCTAAACTTCCG | TACTAACGAACTCCTCGCTCCAA | ACTCCACAATCCCTAAACTTCCA | TACTAACAAACTCCTCACTCCAAA |
| GI29794150_NCI_3R | NT_005403 | CACAACTTATCCCTACTTACCCG | GAACATAAAACCGAAAACACCC | AACACAACTTATCCCTACTTACCCA | AAACATAAAACCAAAAACACCCC |
| GI29794150_TMEFF2_1R | NT_005403 | TTAACCCAAACAATAACCCTACG | TCCTTACTCGAATCTTTACCGAA | ATTAACCCAAACAATAACCCTACA | TCCTTACTCAAATCTTTACCAAATAACC |
| GI29794150_TMEFF2_2R | NT_005403 | TTCTCTCAAACCACTTATCCCG | CCAATCTAACCTTCCAAACACAT | TTTCTCTCAAACCACTTATCCCA | CCAATCTAACCTTCCAAACACAT |
| GI29794150_TMEFF2_3R | NT_005403 | GCAAAACCAAAAACTCCTACCG | ATTAAACCTTCTCTCGTCGCC | CACAAAACCAAAAACTCCTACCA | ATTAAACCTTCTCTCATCACCCC |
| GI29795229_DBCCR1_1R | NT_008470 | CGATCCCTTTAAATACTCGTACG | ATAAACACAACACCCTACACGC | ACACAATCCCTTTAAATACTCATACA | ATAAACACAACACCCTACACACC |
| GI29795229_DBCCR1_2R | NT_008470 | CCCAAAAACACTCAAATACTCG | GACACACACAATACAATCACGCT | CCCCAAAAACACTCAAATACTCA | AACACACACAATACAATCACACTTAA |
| GI29795229_DBCCR1_3R | NT_008470 | ACACAAACGAACCCACACG | ACAACTCCCGAAACAAAACC | ACAAACACAAACAAACCCACACA | ACAACTCCCAAAACAAAACCC |
| GI29795229_TMEFF1_1R | NT_008470 | AACAAACCCAACCTCTTACTCG | CTCCTCGCCTTCCCTTTATATC | AAACAAACCCAACCTCTTACTCA | CTCCTCACCTTCCCTTTATATCC |
| GI29795229_TMEFF1_2R | NT_005470 | GAACCGAAATCTTTATAACGCG | TAACAACGACCACGAACACAA | CAAACAAACCAAAATCTTTATAACACA | TAACAACAACCACAAACACAAAA |
| GI29795229_TMEFF1_3R | NT_008470 | CAAATCACCTTTACCTCTTCCG | AACTCAATTTCTTCCACCTAAAAAC | AACAAATCACCTTTACCTCTTCCA | AACTCAATTTCTTCCACCTAAAAAC |
| GI29796755_H0XA5_1R | NT_007819 | CCGCTAAAAACAAAACTCATCG | CCAACTTCCGACCGAAAACTAC | ACCCACTAAAAACAAAACTCATCA | CCAACTTCCAACCAAAAACTACA |
| GI29796755_HOXA5_2R | NT_007819 | CGATAAACCCTACCTCCAACG | ATAACGCTCGAATCCGACTAAAC | AAACAATAAACCCTACCTCCAACA | ATAACACTCAAATCCAACTAAACAACA |
| GI29796755_HOXA5_3R | NT_007819 | CTACATCCTCGCCGAACG | GCGATCGACAACTAACGACCTAA | ATCCTCTACATCCTCACCAAACA | ACAATCAACAACTAACAACCTAACAA |
| GI29796755_IGFBP1_2R | NT_007819 | ATACTCGCTAAACACAACGCG | ACCTTATAAAAAACACAAACCGC | AACCAATACTCACTAAACACAACACA | ACCTTATAAAAAACACAAACCACACC |
| GI29796755_IGFBP1_3R | NT_007819 | GACCTATACCCTTTATAAAATACGCG | TATATCCAACGAACATCGACCA | CACAACCTATACCCTTTATAAAATACACA | TATATCCAACAAACATCAACCACC |
| GI29796755_IL6_1R | NT_007819 | TCTTAACGCTAACCTCAATAACG | CCTAAACTACACTTTTCCCCCT | TCTACTTCTTAACACTAACCTCAATAACA | CCTAAACTACACTTTTTCCCCCT |
| GI29796755_IL6_2R | NT_007819 | TACCTAACCATCCTCAAATTTCG | ACAATTACTTCAAAACGTCTCCAA | CTACCTAACCATCCTCAAATTTCA | ACAATTACTTCAAAACATCTCCAAA |
| GI29796755_TWIST1_1R | NT_007819 | CTTCCTCGAAATCTAACAATTCG | CCTCCCAAACCATTCAAAAAC | CCTTCCTCAAAATCTAACAATTCA | CCTCCCAAACCATTCAAAAAC |
| GI29796755_TWIST1_2R | NT_007819 | ACTCCGAATAAAACTACCACCG | GACCAAAACAATCTCCTCCGAC | CCACTCCAAATAAAACTACCACCA | AACCAAAACAATCTCCTCCAAC |
| GI29796755_TWIST1_3R | NT_007819 | CCCACCCAATAATCAAATAAACG | ACCCCTTAAAATTCCAAAAACC | CCCACCCAATAATCAAATAAACA | ACCCCTTAAAATTCCAAAAACC |
| GI29796774_TRC8_1R | NT_008046 | ACCCATAATTACCATAATTTCCG | CCACCTCCTCAACTTACTCTAACTTC | CACCCATAATTACCATAATTTCCA | CCACCTCCTCAACTTACTCTAACTTC |
| GI29796774_TRC8_2R | NT_008046 | CACACCCAACCTAATACCACG | ACCGCAAACGCTCCATAA | ACCACACCCAACCTAATACCACA | ACCACAAACACTCCATAAACACA |
| GI29796774_TRC8_3R | NT_008046 | AAACAATAAACGCGAACTCTACG | TTCGCTTAACTAACGACGCAAC | CACAAACAATAAACACAAACTCTACA | TTCACTTAACTAACAACACAACCTCC |
| GI29804415_C4B_1R | NT_007592 | AAACCCAACATAACCCACG | ATACAAAATATTTAAAAACCCTCACAA | AAAACCCAACATAACCCACA | ATACAAAATATTTAAAAACCCTCACAA |
| GI29804415_C4B_2R | NT_007592 | AACCCTATCCTTCCCCG | TAACTCCTCTCATTCCAATACCTAA | AAAATAACCCTATCCTTCCCCA | TAACTCCTCTCATTCCAATACCTAA |
| GI29804415_C4B_3R | NT_007592 | CCATAAACACCCAAATATCCG | AATACCCCCACAACTCTAAACCT | CCCATAAACACCCAAATATCCA | AATACCCCCACAACTCTAAACCT |
| GI29804415_CDKN1A_1R | NT_007592 | TCAACATATTAAAACATATTCCTAACG | CCAAAAAACCAATCAAAACCA | ACTCAACATATTAAAACATATTCCTAACA | CCAAAAAACCAATCAAAACCA |
| GI29804415_CSNK2B_1R | NT_007592 | CCACATTACTTAAAAACTCGAACG | ACGCAAAACTCCGAATTCAAT | CCCACATTACTTAAAAACTCAAACA | ACACAAAACTCCAAATTCAATTTC |
| GI29804415_CSNK2B_2R | NT_007592 | AATCAAATAACCACACGACACG | AAACGCATACGTAACGACAACA | ACAATCAAATAACCACACAACACA | AAACACATACATAACAACAACAACAAC |
| GI29804415_CSNK2B_3R | NT_007592 | AACCTAACCCTTTAAATCTTCCG | GATCCCATTTCGAAATTTCCTCT | CAACCTAACCCTTTAAATCTTCCA | AATCCCATTTCAAAATTTCCTCT |
| GI29804415_EDN1_1R | NT_007592 | CTTTTTCTTAACCCTACCCCCG | ATTATCAAACGACGAACGTCTACC | TCTTTTTCTTAACCCTACCCCCA | ATTATCAAACAACAAACATCTACCTCT |
| GI29804415_EDN1_2R | NT_007592 | TACCCGTACAATACTAAAATCCG | TTCCCCTTTACAAACTACCAACC | CCTACCCATACAATACTAAAATCCA | TTCCCCTTTACAAACTACCAACC |
| GI29804415_EDN1_3R | NT_007592 | ATTATTAATCACCAACAAACAACG | ACAACCGAAAATAAAACCAAACC | TTATATTATTAATCACCAACAAACAACA | ACAACCAAAAATAAAACCAAACC |
| GI29804415_HLA-F_1R | NT_007592 | TCTCCCTTCATTATTCATTCCG | AATCCCAATCCCTTAATTAAACTT | CATCTCCCTTCATTATTCATTCCA | AATCCCAATCCCTTAATTAAACTT |
| GI29804415_HLA-F_2R | NT_007592 | TCTTCCTAAATACTCATAACGCG | CCCCATTTCTCACTCCCATTAA | TCCTTCTTCCTAAATACTCATAACACA | CCCCATTTCTCACTCCCATTAA |
| GI29804415_LY6G6E_1R | NT_007592 | AACCTAACCCAACAAACAACG | AAACCCCCAACCTACCCC | CAACCTAACCCAACAAACAACA | AAACCCCCAACCTACCCC |
| GI29804415_LY6G6E_2R | NT_007592 | TCTACAAATCCCGAAATCTCG | AATACAAATCACCTCTCCCAAA | AACTCTACAAATCCCAAAATCTCA | AATACAAATCACCTCTCCCAAA |
| GI29804415_LY6G6E_3R | NT_007592 | CACAAAACCCAACTATATAAAAACG | ACCCCACCCAACTTCAAA | ACACAAAACCCAACTATATAAAAACA | ACCCCACCCAACTTCAAA |
| GI29804415_NEU1_1R | NT_007592 | TCACTACAACCTCTACCTCCCG | ATTCAAACGATTCTCCTACCTCA | ACTCACTACAACCTCTACCTCCCA | ATTCAAACAATTCTCCTACCTCAAC |
| GI29804415_NEU1_2R | NT_007592 | ACGTAATCACGCAACATCTCG | AACTTACCCTTCCGATTAACCCT | CCACATAATCACACAACATCTCA | AACTTACCCTTCCAATTAACCCTC |
| GI29804415_NEU1_3R | NT_007592 | TCACCATATTAACCAAACTAATCTCG | ACTCCTAACTCAAATAATCCGCC | TTCACCATATTAACCAAACTAATCTCA | ACTCCTAACTCAAATAATCCACCC |
| GI29804415_RDBP_1R | NT_007592 | TTTTAACCCTAACCTTTAACCCG | ATTAAATCCAAACCTCCTTCACC | CTTTTAACCCTAACCTTTAACCCA | ATTAAATCCAAACCTCCTTCACC |
| GI29804415_RDBP_2R | NT_007592 | CCTAACTTTTAACCTTTCCCCG | AATACTACCCCCTCCAAATCCC | CCCTAACTTTTAACCTTTCCCCA | AATACTACCCCCTCCAAATCCC |
| GI29804415_RDBP_3R | NT_007592 | AAACGAAACTTACTTCGCAACG | CTACTTACCGCACTTCCGAACTA | CCAAAACAAAACTTACTTCACAACA | CTACTTACCACACTTCCAAACTACCA |
| GI29804415_TNF_1R | NT_007592 | AAACCCTAAAAACTAAACCCCG | CCCCATACCCCTCAAAACCTAT | AAAACCCTAAAAACTAAACCCCA | CCCCATACCCCTCAAAACCTAT |
| GI29804415_TNF_2R | NT_007592 | AATAAACCCTACACCTTCTATCTCG | TTTCTTCTCCATCGCGAAA | ATAATAAACCCTACACCTTCTATCTCA | TTTCTTCTCCATCACAAAAACAAA |
| GI29804415_TNF_3R | NT_007592 | AAAAAATACAAAACCCACTACCG | TTCCTCCAAATAAACTCATAAATTTC | CAAAAAATACAAAACCCACTACCA | TTCCTCCAAATAAACTCATAAATTTC |
| GI29804415_VEGF_1R | NT_007592 | ACAACCTAAAAATTACCCATCCG | CCCCGAAAACTCTATCCAAAA | CACAACCTAAAAATTACCCATCCA | CCCCAAAAACTCTATCCAAAAAC |
| GI29804415_VEGF_2R | NT_007592 | CCCTTCATTACGACGAACTACG | ACCAAACTTCACTAAACGTCCG | TCCCCTTCATTACAACAAACTACA | ACCAAACTTCACTAAACATCCACA |
| GI29804415_VEGF_3R | NT_007592 | AACGACTCTCAAACCCTATCCG | ACGTAACCTCACTTTCCTACTCC | AAACAACTCTCAAACCCTATCCA | ACATAACCTCACTTTCCTACTCCCT |
| GI29807454_WT1_1R | NT_009237 | CTACTCCCACCGCATTCG | CCCTACCCGAACTCACTACTTAC | CATCTCTACTCCCACCACATTCA | CCCTACCCAAACTCACTACTTACC |
| GI29824571_MOS_1R | NT_008183 | CAATCATATTTCCAAAATCCCG | GATTTCCCCTAATCTCTTCATTCA | TCAATCATATTTCCAAAATCCCA | AATTTCCCCTAATCTCTTCATTCA |
| GI29789877_CSF1_2R | NT_019273 | TCCTAATCACCCTCTATCTTCTACG | TACACTTCCAAACCTTCAACAAAC | TTCCTAATCACCCTCTATCTTCTACA | TACACTTCCAAACCTTCAACAAAC |
| GI29789877_CSF1_3R | NT_019273 | AAAAAATCACCCTAACCAAACG | CAAACACACACATACACACACACA | ATAAAAAATCACCCTAACCAAACA | CAAACACACACATACACACACACA |
| GI29789881_MTHFR_1R | NT_021937 | AAATAACCTAATCACTTCAAACCG | AACATTATAATTCCAACCAAAAATACTC | AATAAATAACCTAATCACTTCAAACCA | AACATTATAATTCCAACCAAAAATACTC |
| GI29791372_MLH1_1R | NT_022517 | CCCCTTACGACCTTTCTAACG | TAACCCTACTCTTATAACCTCCCG | TCTCCCCTTACAACCTTTCTAACA | TAACCCTACTCTTATAACCTCCCA |
| GI29791372_MLH1_2R | NT_022517 | CACCTCAATACCTCGTACTCACG | TCTTCCTTCAACTATAACTTACGCC | TCACCTCAATACCTCATACTCACA | TCTTCCTTCAACTATAACTTACACCA |
| GI29791372_MLH1_3R | NT_022517 | CACATACCGCTCGTAATATTCG | ACTCAACCTCGTAATAACGCCTA | CCACCACATACCACTCATAATATTCA | ACTCAACCTCATAATAACACCTAACA |
| GI29791392_EGR4_1R | NT_022184 | TAACACTCAATCCCCCTTAACG | CAATTTCTCACCTATACATTTCCTATAA | TTAACACTCAATCCCCCTTAACA | CAATTTCTCACCTATACATTTCCTATAA |
| GI29791392_EGR4_2R | NT_022184 | AAACAAATTAAACCCAACACG | TCCTCCCCTACTACCCCTCG | AAAACAAATTCAAACCCAACACA | TCCTCCCCTACTACCCCTCAAC |
| GI29791392_EGR4_3R | NT_022184 | ACCCGATAAAACGCAACG | CCCAAACTAACGCATCCG | CAATCAACCCAATAAAACACAACA | CCCAAACTAACACATCCACAAC |
| GI29791392_POMC_1R | NT_022184 | AACGACCAAATACGCCTTCG | CAAAACAATACTAATTCCAACCCC | AACAAACAACCAAATACACCTTCA | CAAAACAATACTAATTCCAACCCC |
| GI29791392_POMC_2R | NT_022184 | CACGAAAATACTAAACCTCCCG | CCGTTCTAAACGAAAACCCAAC | CACACAAAAATACTAAACCTCCCA | CCATTCTAAACAAAAACCCAACA |
| GI29791392_POMC_3R | NT_022184 | ACGCAAATAACTTCACCCTCG | CTCAACGACCTCAAAAACTACCC | CACACACAAATAACTTCACCCTCA | CTCAACAACCTCAAAAACTACCC |
| GI29791392_SFTPB_1R | NT_022184 | CCCCTTATAACTAAACGAAACG | AAAAACTCTATAAAAATAACAACGACCT | CATAACCCCTTATAACTAAACAAAACA | AAAAACTCTATAAAAATAACAACAACCTC |
| GI29791392_SFTPB_2R | NT_022184 | TCCTACAAAACCCCCACG | CCCGCCCAACTATAAAAAA | ACTCCTACAAAACCCCCACA | CCCACCCAACTATAAAAAACCA |
| GI29791392_TGFA_1R | NT_022184 | ATAACCGCCTTCCTATTTCCG | CCGACGAACAACGCTACG | ACAATAACCACCTTCCTATTTCCA | CCAACAAACAACACTACAAAACAA |
| GI29791392_TGFA_2R | NT_022184 | CCGCCTAAAACCTAAAAACCG | CACTACGACCCAAAACAATCC | CACCACCTAAAACCTAAAAACCA | CACTACAACCCAAAACAATCCAA |
| GI29791392_TGFA_3R | NT_022184 | AAATCTTAACAAACGACCGACG | AACTCACAAATCCCTTTCCTAAC | CCAAATCTTAACAAACAACCAACA | AACTCACAAATCCCTTTCCTAAC |
| GI29794065_N33_1R | NT_015280 | CTCTCCTCAACGCTAATCCG | AAAAAACAAACTCCGAACGAAA | CTCCTCTCCTCAACACTAATCCA | AAAAAACAAACTCCAAACAAAAACA |
| GI29794065_N33_2R | NT_015280 | AAAATAACAACCTAACCCCTACG | TCCCCAACAATCTCTATTCCC | AAAAATAACAACCTAACCCCTACA | TCCCCAACAATCTCTATTCCC |
| GI29794065_N33_3R | NT_015280 | CCGAAACCTAACTCCCTCG | CACGCCCACTTCCTACCC | CCCAAAACCTAACTCCCTCA | CACACCCACTTCCTACCCC |
| GI29798364_UBB_1R | NT_010718 | AATCAACGCCGACCTCG | CTTCGCAAACCTAACCAATCAAT | CACCAATCAACACCAACCTCA | CTTCACAAACCTAACCAATCAAT |
| GI29798364_UBB_2R | NT_010718 | CGCTCAATTACTTAACAACCTCG | CGCTAAACCACCCCAAATAAAAC | CACACTCAATTACTTAACAACCTCA | CACTAAACCACCCCAAATAAAAC |
| GI29798364_UBB_3R | NT_010718 | GACTCATCGCAACCTCCG | CTCCCGAATTCAAACGATTCT | ATCTCAACTCATCACAACCTCCA | CTCCCAAATTCAAACAATTCTCC |
| GI29798595_ERBB2_1R | NT_010755 | CTCTACCCCCTCCCCCG | AATCCGAAATAAATTCCCTAAACT | ATAACTCTACCCCCTCCCCCA | AATCCAAAATAAATTCCCTAAACTACC |
| GI29798595_ERBB2_2R | NT_010755 | AACGCCTAAATTACCTACAACCG | TCTCACACTTTTCCTCGAAAAAT | CAAAACACCTAAATTACCTACAACCA | TCTCACACTTTTCCTCAAAAAATC |
| GI29798595_ERBB2_3R | NT_010755 | ACTTCACTTTCTCCCTCTCTTCG | GCAAACCTAAATACGTCCCTCCT | AACTTCACTTTCTCCCTCTCTTCA | ACAAACCTAAATACATCCCTCCT |
| GI29799031_ATP5G1_1R | NT_010783 | TCAAACACATAACTTTAACCCCG | ACCTTCATTCGAACGCTTAAAC | AATCAAACACATAACTTTAACCCCA | ACCTTCATTCAAACACTTAAACCAC |
| GI29799031_ATP5G1_2R | NT_010783 | TCCCCACTCTTATTAACTTTCCG | AACTACTTCCTCTCCCTCCACAA | TTCCCCACTCTTATTAACTTTCCA | AACTACTTCCTCTCCCTCCACAA |
| GI29799031_ATP5G1_3R | NT_010783 | CGCCAACTCTATAATCGAACG | TTCAAAAATCGACCAATCGTAAT | TCCCACCAACTCTATAATCAAACA | TTCAAAAATCAACCAATCATAATCC |
| GI29799031_NME1_1R | NT_010783 | GAATTCTCTAACTCACTTCCTTCG | TCGCGAACAAATTAACTTCCC | CAAATTCTCTAACTCACTTCCTTCA | TCACAAACAAATTAACTTCCCAAA |
| GI29799031_NME1_2R | NT_010783 | GCTAACTTTTTCAAACCTTTCCG | TTAACTACTTACTTTCTTCTCTCCACCC | CACTAACTTTTTCAAACCTTTCCA | TTAACTACTTACTTTCTTCTCTCCACCC |
| GI29799031_NME1_3R | NT_010783 | AACGTATAAACGCCACCTCTCG | AAAACCAATTTACTCGCGAACG | CACAACATATAAACACCACCTCTCA | AAAACCAATTTACTCACAAACAAAA |
| GI29799354_NF1_1R | NT_010799 | CCCGAATCAACTCTAACACTCG | CAACTAAACCCAACGCCAATCTA | CTCCCAAATCAACTCTAACACTCA | CAACTAAACCCAACACCAATCTAA |
| GI29799354_NF1_2R | NT_010799 | GCCCTAACTTCCAACTCCG | AAACAATCCAAACCCGAAAAC | AAACACCCTAACTTCCAACTCCA | AAACAATCCAAACCCAAAAACC |
| GI29799354_NF1_3R | NT_010799 | TCATTAATAAAACCGACCGACG | GAACGCATACGCGACAAAC | TTTTCATTAATAAAACCAACCAACA | AAACACATACACAACAAACCACC |
| GI29800594_APOC2_1R | NT_011109 | AAACCTACTAACTCCACCCACG | TCCTACCCTCACCCTCCTAAA | AAAAACCTACTAACTCCACCCACA | TCCTACCCTCACCCTCCTAAA |
| GI29800594_AP0C2_2R | NT_011109 | AACCCAACCTCTATCGAAAACG | ATTCTCAAAATAAAAATTCCCTATCA | CAACCCAACCTCTATCAAAAACA | ATTCTCAAAATAAAAATTCCCTATCA |
| GI29800594_APOC2_3R | NT_011109 | TCCCTATAACGTAACCTTAAAAAACG | CATTACCCTTTCTATCCCCACC | TTCCCTATAACATAACCTTAAAAAACA | CATTACCCTTTCTATCCCCACC |
| GI29800594_KLK10_3R | NT_011109 | GAATCACGAAATCAAAAATTCG | AACCAACCTAACCAACATAATAAAACC | AACAAATCACAAAATCAAAAATTCA | AACCAACCTAACCAACATAATAAAACC |
| GI29800594_TSLL2_2R | NT_011109 | ATAACTCAACTCACAACCTCTCG | TAAACTCCAAACACCCACCTACC | CAATAACTCAACTCACAACCTCTCA | TAAACTCCAAACACCCACCTACC |
| GI29800594_TSLL2_3R | NT_011109 | CTTAACCTACCTCGACCTCCG | ACCCTCCGAACCTCCAACTAC | ATCCTTAACCTACCTCAACCTCCA | ACCCTCCAAACCTCCAACTAC |
| GI29801019_STK11_1R | NT_011255 | ACCAACCTAAAAACTAAAAAATAACG | CTACATCTACTCATTTCCTCCCA | AACCAACCTAAAAACTAAAAAATAACA | CTACATCTACTCATTTCCTCCCA |
| GI29801019_STK11_2R | NT_011255 | AAATCTTACTATATCTCCCAAACCG | AATACAATAACATAACCTCTACCTCCC | AAAATCTTACTATATCTCCCAAACCA | AATACAATAACATAACCTCTACCTCCC |
| GI29801019_STK11_3R | NT_011255 | CAAAAATCATACCATTACACTCCG | CCTAAAAAACACAACAAAACTCCA | CAAAAATCATACCATTACACTCCA | CCTAAAAAACACAACAAAACTCCA |
| GI29801560_CDKN2D_1R | NT_011295 | ATTACCGACGATCCACCG | TTACCACACTCTAACCAATCAAAA | ACCATTACCAACAATCCACCA | TTACCACACTCTAACCAATCAAAA |
| GI29801560_CDKN2D_2R | NT_011295 | ATTAAACCAACCTTCTTTCCCG | CTACCGAATTCATTTAAAAACCG | AATTAAACCAACCTTCTTTCCCA | CTACCAAATTCATYTAAAAACCAAAA |
| GI29801560_CDKN2D_3R | NT_011295 | CATCCACTCCGTCTCTCCG | TTCCCTTTCTTCACGATACTTAACA | CTCATCCACTCCATCTCTCCA | TTCCCTTTCTTCACAATACTTAACA |
| GI29801560_ICAM1_1R | NT_011295 | ATCCTCCCTCGCTAACCG | TTCAACTCCGAAATTTCCAAACT | AAATCATCCTCCCTCACTAACCA | TTCAACTCCAAAATTTCCAAACT |
| GI29801560_ICAM1_2R | NT_011295 | CGAACGTAATAAAACCGCG | TTCGTCACTCCCACGATTAAC | TCCCCAAACATAATAAAACCACA | TTCATCACTCCCACAATTAACAA |
| GI29801560_ICAM1_3R | NT_011295 | CCTAACTAACACGAACATTTCTCG | AAAACGACCAAAACAAAACCAA | CACCTAACTAACACAAACATTTCTCA | AAAACAACCAAAACAAAACCAAA |
| GI29801767_THBS1_1R | NT_010194 | CCAATCTCTAATATCCACCTCTCG | CATCAACCAAACATTCCGAAA | CCAATCTCTAATATCCACCTCTCA | CATCAACCAAACATTCCAAAAA |
| GI29801767_THBS1_2R | NT_010194 | CAACTCAAAACAAACGCTCG | AAAACGCGTATCCTCACCC | AAACCAACTCAAAACAAACACTCA | AAAACACATATCCTCACCCCAC |
| GI29801767_THBS1_3R | NT_010194 | TTCGTTAAAATACCTACCCCTCG | ACCCTCGCTAACTCTCCTCC | CATTCATTAAAATACCTACCCCTCA | ACCCTCACTAACTCTCCTCCAC |
| GI29801784_ELK1_1R | NT_011568 | CCTTTTCAATTACTCACAATCCG | CAATCCTTAAACCAATCGACGTA | ACCTTTTCAATTACTCACAATCCA | CAATCCTTAAACCAATCAACATAAA |
| GI29801784_ELK1_2R | NT_011568 | GCTTTAACCAATCAACGAACG | CGAAACATTAAACTCCTCCTCCT | AACACTTTAACCAATCAACAAACA | CAAAACATTAAACTCCTCCTCCTC |
| GI29801784_ELK1_3R | NT_011568 | CATTTCTATACAAACCCTACTTCCG | TAAACAACATAACGTACGACACCG | CCATTTCTATACAAACCCTACTTCCA | TAAACAACATAACATACAACACCACC |
| GI29802832_FMR1_1R | NT_011681 | AAAATTCGACCTCAATCAAACG | TCAACTCCGTTTCGATTTCACT | CAAAAATTCAACCTCAATCAAACA | TCAACTCCATTTCAATTTCACTTC |
| GI29802832_FMR1_2R | NT_011681 | AATAAACGTTCTAACCCTCGCG | AACAATACGACCTATCACCGC | CAAAAATAAACATTCTAACCCTCACA | AACAATACAACCTATCACCACCCT |
| GI29802832_FMR1_3R | NT_011681 | TCCCGCTCAATCAAACTACG | TACTTTAAACCGAACCAAACCAA | ACCTCCCACTCAATCAAACTACA | TACTTTAAACCAAACCAAACCAAA |
| GI29802923_APAF1_1R | NT_019546 | CACTACGATATTACTCCAAATCCG | AAAAATTCAAACTCCCGAACG | TCCACTACAATATTACTCCAAATCCA | AAAAATTCAAACTCCCAAACACA |
| GI29802923_APAF1_2R | NT_019546 | ACAAAAACTCCCTTAAACCCCG | CTTCTTCCGACTCTTCACCTCAA | AACAAAAACTCCCTTAAACCCCA | CTTCTTCCAACTCTTCACCTCAA |
| GI29802923_APAF1_3R | NT_019546 | CGAATCCGACATTAATAAAAACG | GACGCGTCCCTAAAACTTAACC | CCCAAATCCAACATTAATAAAAACA | AACACATCCCTAAAACTTAACCACA |
| GI29803889_GPC3_1R | NT_011786 | CCTAACTTAAATCCCCCTCCG | TTCGCCACAAACCTTCTTTC | CCTAACTTAAATCCCCCTCCA | TTCACCACAAACCTTCTTTCAAC |
| GI29803889_GPC3_2R | NT_011786 | ACGCCCTATATAAAACGACTACG | ACGAACAACTAAACTCGACTACCG | CCAAAACACCCTATATAAAACAACTACA | ACAAACAACTAAACTCAACTACCAAA |
| GI29803889_GPC3_3R | NT_011786 | CAAAACCAATCAAAACGAACG | CTACTAAAAAACCAATCAACGCG | CCAAAACCAATCAAAACAAACA | CTACTAAAAAACCAATCAACACACTCA |
| GI29805200_CCND2_1R | NT_009759 | CCAACTTTAACTTCTTCAAAACG | ATCCTAATCCTCCTACCCTTATATTT | CTCCAACTTTAACTTCTTCAAAACA | ATCCTAATCCTCCTACCCTTATATTT |
| GI29805200_CCND2_3R | NT_009759 | TCTCCGCTCTAAAACGATAACG | AAACTAACTAAACAATTAACTCTCCTCCC | CTCTTCTCCACTCTAAAACAATAACA | AAACTAACTAAACAATTAACTCTCCTCCC |
| GI29806267_SOD1_1R | NT_011512 | AAAAATTATTTTCTCCACATTTCG | AATTCTAAACGTTTCCCGACTAC | AAAAAATTATTTTCTCCACATTTCA | AATTCTAAACATTTCCCAACTACAAA |
| GI29806267_SOD1_2R | NT_011512 | ACATCATTTTACCAATTTCGCG | ACTACAACCGACGAACCACG | TTTACATCATTTTACCAATTTCACA | ACTACAACCAACAAACCACACC |
| GI29806267_SOD1_3R | NT_011512 | AATCATTCCCGACCACTCG | GACCCGAAACTACCGCAAA | CAATCATTCCCAACCACTCA | AACCCAAAACTACCACAAAAAAC |
| GI29806588_GP1BB_1R | NT_011519 | CCAACACCGCTATAATATACCG | AATCCTAAACCTAAACCTCCCGA | CCCCAACACCACTATAATATACCA | AATCCTAAACCTAAACCTCCCAA |
| GI29806588_GP1BB_2R | NT_011519 | AACCCCATTTTCTATCGAAACG | ACCGAATCTTCCCTTATCCC | AAAACCCCATTTTCTATCAAAACA | ACCAAATCTTCCCTTATCCCC |
| GI29806588_GP1BB_3R | NT_011519 | AATATAAACGATCACGACCCCG | TCCTTACAACCTCTAACCTCCTT | AAAATATAAACAATCACAACCCCA | TCCTTACAACCTCTAACCTCCTT |
| GI29807454_WT1_2R | NT_009237 | TACGCTTTCCTAAAATTCCCG | CCTCTTAAAACCTACCTACCCCTC | TTTCTACACTTTCCTAAAATTCCCA | CCTCTTAAAACCTACCTACCCCTC |
| GI29807454_WT1_3R | NT_009237 | AACGTCCCTCAATTAAATAACCG | AAATTACACCAATTATAAACCGAACG | AAACATCCCTCAATTAAATAACCA | AAATTACACCAATTATAAACCAAACA |
| GI29808062_SOCS1_1R | NT_010393 | CCTCTCTTCTAAACCCTCCCG | GAAACCCTCTACCCGCCTATT | CCTCTCTTCTAAACCCTCCCA | AAAACCCTCTACCCACCTATTCA |
| GI29808062_SOCS1_2R | NT_010393 | CCGAAAAATAACCGAAAAACG | AAAATCGAAACCAAAACCCC | AAATCCAAAAAATAACCAAAAAACA | AAAATCAAAACCAAAACCCCTC |
| GI29808062_SOCS1_3R | NT_010393 | GCCGAACAAAACGAACTACG | CCGTAACAACTACACGACTCCTAA | CAAACACCAAACAAAACAAACTACA | CCATAACAACTACACAACTCCTAACC |
| GI29808625_CDH3_2R | NT_010498 | GATCTCGACTCACTACAACCTCG | CCTCCCGAACTAAAACGATTCT | CAATCTCAACTCACTACAACCTCA | CCTCCCAAACTAAAACAATTCTC |
| GI29808625_CDH3_3R | NT_010498 | CGCGCTACTACACTCCTAAACG | CAAAACGAAACCCTATCTCCAA | CAAAATCACACTACTACACTCCTAAACA | CAAAACAAAACCCTATCTCCAAA |
| GI29808952_TUBB4_1R | NT_010542 | CCGATTTAAAAACCAACCCG | ACCATCCCAACTCCCTATCTTT | ACACCAATTTAAAAACCAACCCA | ACCATCCCAACTCCCTATCTTT |
| GI29808952_TUBB4_2R | NT_010542 | CGATACGAAACCTACGAACCG | ACGAAACTCTACGACGACGC | CACACAATACAAAACCTACAAACCA | ACAAAACTCTACAACAACACCTCC |
| GI29808952_TUBB4_3R | NT_010542 | GCGATAACATCAACCGATACG | AAAACGAAACCGCGACTATAAA | CCCACAATAACATCAACCAATACA | AAAACAAAACCACAACTATAAAAACA |
| GI29823164_ADCYAP1_1R | NT_010859 | AACGCTTCTTCAAATTCCTACG | TAAAATCACCACCCGAAAAACAT | CAAAACACTTCTTCAAATTCCTACA | TAAAATCACCACCCAAAAAACAT |
| GI29823164_ADCYAP1_2R | NT_010859 | CAACAATCGAATAAACCGATCG | TTTAATAAAATTTTCCCTCCCTTACC | AAATCAACAATCAAATAAACCAATCA | TTTAATAAAATTTTCCCTCCCTTACC |
| GI29823164_ADCYAP1_3R | NT_010859 | TACTACTCCCGCTAATTCCTACG | CTTCTACTCAAACACCAACGCC | TCCTACTACTCCCACTAATTCCTACA | CTTCTACTCAAACACCAACACCA |
| GI29823164_MC2R_1R | NT_010859 | AAAAAATCAATCAAATTTTCCG | AAAATCAAATCCAAATAACATCCC | CCTAAAAAATCAATCAAATTTTCCA | AAAATCAAATCCAAATAACATCCC |
| GI29823167_ATP5A1_1R | NT_010966 | CCTCCTACGACTCCATAACTACG | CTCCCGCCACTTTACTAAAAACT | TCTCCTCCTACAACTCCATAACTACA | CTCCCACCACTTTACTAAAAACTCC |
| GI29823167_ATP5A1_2R | NT_010966 | CTAACATTACAAACCTCGCTTCG | TACCACTTCCCAACTCTTCCC | AACTCTAACATTACAAACCTCACTTCA | TACCACTTCCCAACTCTTCCC |
| GI29823167_ATP5A1_3R | NT_010966 | CTTCCACATCTAAACTACTTCCG | GCCCTACATCCCAACACTAAAA | CAACTTCCACATCTAAACTACTTCCA | ACCCTACATCCCAACACTAAAAA |
| GI17458490_PLAGL2_1R | NT_028392 | TACTACTACTTCTACCGCTCCCG | AAATACCTACGAACTCCTAAAACCC | TTCCTACTACTACTTCTACCACTCCCA | AAATACCTACAAACTCCTAAAACCCA |
| GI17458490_PLAGL2_2R | NT_028392 | TCAACGACCGTACCCACG | GACGACGCCCATATCGAC | ACACTCAACAACCATACCCACA | AACAACACCCATATCAACCCA |
| GI17458490_PLAGL2_3R | NT_028392 | ACTTAAACCTCAAACAAATCACG | AACCTCTCCCAACCTCAATTTT | AACTTAAACCTCAAACAAATCACA | AACCTCTCCCAACCTCAATTTT |
| GI29732427_ABL1_1R | NT_035014 | TTCCAACAAATTTCCTACCG | ACTAATAACCGCTCTCGCTTCT | ATCTTCCAACAAATTTCCTACCA | ACTAATAACCACTCTCACTTCTCTACTTT |
| GI29732427_ABL1_2R | NT_035014 | ACATTCCTTTTCGTCAAAATCG | AAACAAACTCGCTAAAATCTAAATAAC | CAATACATTCCTTTTCATCAAAATCA | AAACAAACTCACTAAAATCTAAATAACCC |
| GI29732427_ABL1_3R | NT_035014 | CATTACCAACAACTTTTAAATCCG | TTTTTAAAAAACCTATAAAACCAATCC | CATTACCAACAACTTTTAAATCCA | TTTTTAAAAAACCTATAAAACCAATCC |
| GI29736559_CRIP1_1R | NT_026437 | ACAAACTCCGCCTAACACCG | AACCATCCTCCGCCTCA | AAAACAAACTCCACCTAACACCA | AACCATCCTCCACCTCAACTTTA |
| GI29736559_CRIP1_2R | NT_026437 | CCAAACGCCTATACCTTAACCG | CCTACAACCCTACTACCCCTACC | ATACCAAACACCTATACCTTAACCA | CCTACAACCCTACTACCCCTACC |
| GI29736559_CRIP1_3R | NT_026437 | TAAAACGACCAAAACACAAACG | CTAACACAAAAACCACACTCCAA | CTATAAAACAACCAAAACACAAACA | CTAACACAAAAACCACACTCCAA |
| GI29736559_DAD1_1R | NT_026437 | TCAAAAACTAACAAACCAAACG | CCCAAAAACTAAACTTCCTCTAAA | TTTCAAAAACTAACAAACCAAACA | CCCAAAAACTAAACTTCCTCTAAA |
| GI29736559_DAD1_2R | NT_026437 | ATCCCCTCAACCCAACG | ACACTACCACCACCACCATC | ATCCCCTCAACCCAACA | ACACTACCACCACCACCATC |
| GI29736559_FOS_1R | NT_026437 | CCTTAACGCGTATCCTAATCTCG | AAACATTTCGCAATTCCTATCTC | CCACCTTAACACATATCCTAATCTCA | AAACATTTCACAATTCCTATCTCAAA |
| GI29736559_FOS_2R | NT_026437 | AATCCGCATTAAACCAAATACG | ATATTCTCTCTCATTCTACGCCG | CAAAAATCCACATTAAACCAAATACA | ATATTCTCTCTCATTCTACACCATTCC |
| GI29736559_FOS_3R | NT_026437 | GAAAACGGAAACTTAAATCCTCG | AATCCTATACTCGATACCGTTTCTC | CAAAAACCAAAACTTAAATCCTCA | AATCCTATACTCAATACCATTTCTCC |
| GI29736559_HSPA2_1R | NT_026437 | AAAACCCGAAAACCCCG | ACAACCACACCAAACCCTATATCT | ACAAAACCCAAAAACCCCA | ACAACCACACCAAACCCTATATCT |
| GI29736559_HSPA2_2R | NT_026437 | CCTATCGACCTCTAATACACTCG | ACTTCCAACCCTTAAAACAAACA | ACTAACCTATCAACCTCTAATACACTCA | ACTTCCAACCCTTAAAACAAACA |
| GI29738185_ESR1_1R | NT_023451 | CAAACACAACTCGATTTAAAACG | TCCCAAAAAACAACTTCCCTAA | TCCAAACACAACTCAATTTAAAACA | TCCCAAAAAACAACTTCCCTAA |
| GI29738185_ESR1_2R | NT_023451 | CCTAAATCCGTCTTTCGCG | TTATTTTAAACCCAATCTTCCCT | AACCCCTAAATCCATCTTTCACA | TTATTTTAAACCCAATCTTCCCT |
| GI29738185_ESR1_3R | NT_023451 | CCCATTCTATCTACCCTATCTCG | TTACAATATAATCCTCCCCAAAATC | CCCCATTCTATCTACCCTATCTCA | TTACAATATAATCCTCCCCAAAATC |
| GI29738645_RB1_1R | NT_024524 | TAAAAAACGCCTAAACCCACG | CAAATTTCCCAATTTAATTCCTCA | CCTAAAAAACACCTAAACCCACA | CAAATTTCCCAATTTAATTCCTCA |
| GI29738645_RB1_3R | NT_024524 | AACGAAAATAACGTTTTCCCG | GATTAAACGCGACGCTCAATTAC | ACAAACAAAAATAACATTTTCCCA | AATTAAACACAACACTCAATTACCAAA |
| GI29738863_GDF10_1R | NT_030772 | GCAACCAAAACAAACTACGACG | CGACACACAAAAACCGACTAC | TCACAACCAAAACAAACTACAACA | CAACACACAAAAACCAACTACAAA |
| GI29738863_GDF10_2R | NT_030772 | TCATCTTACAATCACATAACCATCG | ATACCATTACAAAAACAACCCAACA | TCATCTTACAATCACATAACCATCA | ATACCATTACAAAAACAACCCAACA |
| GI29738863_GDF10_3R | NT_030772 | GCTAACTCCGAAACAACTAAACG | ACACATATCCCCTCTCTAATCCTC | CATACACTAACTCCAAAACAACTAAACA | ACACATATCCCCTCTCTAATCCTC |
| GI29739550_OAT_1R | NT_035040 | CTCTACCCCAACCACAACTACG | AAACCCCATACCCTTAAACCCTA | ACTCTACCCCAACCACAACTACA | AAACCCCATACCCTTAAACCCTA |
| GI29739550_OAT_2R | NT_035040 | CCCAACAAACTTTTCCTTTTCG | ACCTCAATCTTCTCGTCAACAAA | ACCCAACAAACTTTTCCTTTTCA | ACCTCAATCTTCTCATCAACAAA |
| GI29739550_OAT_3R | NT_035040 | GCGATTAATATCCTACCCTCCG | CCCAACCAATAAACGACGAAA | CACACAATTAATATCCTACCCTCCA | CCCAACCAATAAACAACAAAAAT |
| GI29759893_CTNNB1_1R | NT_037565 | CCCGATACAAACCACAACG | CCTCACGAACTACCCTCAAAC | TCCCCAATACAAACCACAACA | CCTCACAAACTACCCTCAAACC |
| GI29791372_RARB_1R | NT_022517 | GAAACGCTACTCCTAACTCACG | TAAACACCAACTTCTCTCCCTTT | AACCAAAACACTACTCCTAACTCACA | TAAACACCAACTTCTCTCCCTTT |
| GI29791372_RARB_2R | NT_022517 | GTAAACCAAAAACAACGTCCCG | CTCCTCCCCTACTCATTTTAAAA | CATAAACCAAAAACAACATCCCA | CTCCTCCCCTACTCATTTTAAAA |
| GI29791372_RARB_3R | NT_022517 | AACGCAAACGAAACACCG | TTTCCAAACTAAACCGCCG | AAACAAACACAAACAAAACACCA | TTTCCAAACTAAACCACCACAAA |
| GI29791375_ARHI_1R | NT_032977 | ACCCTATAAACCTAATCGTTAACG | TAACACCTAATTTTATATACCCCAACC | CAATACCCTATAAACCTAATCATTAACA | TAACACCTAATTTTATATACCCCAACC |
| GI29791375_ARHI_2R | NT_032977 | GTCTACCCTCCACATCTCCG | ACTCCGTCTAACTTCTTTCTCCC | TACATCTACCCTCCACATCTCCA | ACTCCATCTAACTTCTTTCTCCCTT |
| GI29791375_ARHI_3R | NT_032977 | AACGCTCGATAAACGATAATACG | AACTTTCAATACATCCGCCG | CCCAAAACACTCAATAAACAATAATACA | AACTTTCAATACATCCACCACCA |
| GI29791382_SFN_2R | NT_037485 | ACCCCTAACTACTTCAACCTCG | CAAAACCCATAAAAATTCAAAAA | AACCCCTAACTACTTCAACCTCA | CAAAACCCATAAAAATTCAAAAA |
| GI29791382_SFN_3R | NT_037485 | CAAATCTTAAACCCTAATCCCG | CCCCTACTCCTCCCCAC | ACAAATCTTAAACCCTAATCCCA | CCCCTACTCCTCCCCAC |
| GI29794267_SFTPC_1R | NT_023666 | CCCTTCTATATAAACCTCCCCG | TTCCTCCTCCAACCCCTC | CCCTTCTATATAAACCTCCCCA | TTCCTCCTCCAACCCCTC |
| GI29794267_SFTPC_2R | NT_023666 | AAACAACTATCCCCACCCG | CCCTAACACGACTCTACCCAA | AAACAACTATCCCCACCCA | CCCTAACACAACTCTACCCAAT |
| GI29794267_SFTPC_3R | NT_023666 | CCTTACTACCACACATTTAACCG | CCTCCCCATACCAACTTAAAA | TCCTTACTACCACACATTTAACCA | CCTCCCCATACCAACTTAAAA |
| GI29794674_EGFR_1R | NT_033968 | TCAAATCTTATCAAACACCCTCG | AATCATCTAAAATAAAAACACCCAAC | TTCAAATCTTATCAAACACCCTCA | AATCATCTAAAATAAAAACACCCAAC |
| GI29794674_EGFR_2R | NT_033968 | AAACCCAACCTATATCCAAATCG | ACCAAATCTATACCAAAATCCCC | CAAACCCAACCTATATCCAAATCA | ACCAAATCTATACCAAAATCCCC |
| GI29794674_EGFR_3R | NT_033968 | AATACTAAAAACGCCCCTCTCG | AAATTAACTCCTCAAAACACCCG | AACAATACTAAAAACACCCCTCTCA | AAATTAACTCCTCAAAACACCCAC |
| GI29796148_TERT_1R | NT_023089 | CTTTAACCGCTAACCTAATCCG | AAACCCAAAACTACCTCCAAATC | CCCTTTAACCACTAACCTAATCCA | AAACCCAAAACTACCTCCAAATC |
| GI29796148_TERT_2R | NT_023089 | CCCGAAACAACTACGCTATCG | AACCAAACCGAACTCCCAATAA | CCACCCAAAACAACTACACTATCA | AACCAAACCAAACTCCCAATAAA |
| GI29796148_TERT_3R | NT_023089 | AATTACCCCACAACCTAAACCG | TTCGACCTCTCTCCGCTAAA | AAATTACCCCACAACCTAAACCA | TTCAACCTCTCTCCACTAAAACC |
| GI29797939_IL13_1R | NT_034772 | CGCAAATCCCGCTTATCG | ACCCATCTCCCGTTACATAAAAC | AACCACACAAATCCCACTTATCA | ACCCATCTCCCATTACATAAAAC |
| GI29797939_IL13_2R | NT_034772 | GCCAAACAAAACAAAAACCG | CTAAACACCCGAACCAACGACT | AACACCAAACAAAACAAAAACCA | CTAAACACCCAAACCAACAACTC |
| GI29797939_IL13_3R | NT_034772 | GCAACGCATTACAAACAAATACG | ACCACATACGCCAAATACCC | CACACAACACATTACAAACAAATACA | ACCACATACACCAAATACCCAAA |
| GI29797939_LOX_1R | NT_034772 | CACTCTCTCGCTTTTATAAAAAACG | CCTCAACAAATAAACCCCAAAAT | CACACTCTCTCACTTTTATAAAAAACA | CCTCAACAAATAAACCCCAAAAT |
| GI29797939_LOX_2R | NT_034772 | ATTACACAAACCGTTCTAACCCG | CCGCCCCTCAACTATTTATTC | AAATTACACAAACCATTCTAACCCA | CCACCCCTCAACTATTTATTCAC |
| GI29797939_LOX_3R | NT_034772 | GAAAAACTATCCGCCTTACACG | TTCCAATCGCATTACGTAAACA | AAACAAAAAACTATCCACCTTACACA | TTCCAATCACATTACATAAACAAATAA |
| GI29800185_SFTPD_1R | NT_030059 | TCACTCTTATCCCCACCTACG | TCTAAAAACCTACCACTAATATTCACAA | ACTCACTCTTATCCCCACCTACA | TCTAAAAACCTACCACTAATATTCACAA |
| GI29800185_SNCG_1R | NT_030059 | CCACCAACATCCTAAATACCG | TAAACCCTAAACTACCATAAAACCG | CCCACCAACATCCTAAATACCA | TAAACCCTAAACTACCATAAAACCA |
| GI29800185_SNCG_2R | NT_030059 | ACACAACCCAAACCGCG | AAAATTAACTACTCTCACCTAACAAACC | AACTACACAACCCAAACCACA | AAAATTAACTACTCTCACCTAACAAACC |
| GI29800185_SNCG_3R | NT_030059 | ACCTCAAATCCTCCAACCG | TTTCATAACAACCCAAAATCCA | ACCTCAAATCCTCCAACCA | TTTCATAACAACCCAAAATCCA |
| GI29801752_PLAGL1_1R | NT_025741 | CCCAAATACAACTACCCAAACG | AAACACTAACCGTAAACCAAACAC | ACCCAAATACAACTACCCAAACA | AAACACTAACCATAAACCAAACACT |
| GI29801752_PLAGL1_2R | NT_025741 | AACCGACTCGAATCTACCTACG | CAACGCTATACCTAAACGACCTT | CCAAACCAACTCAAATCTACCTACA | CAACACTATACCTAAACAACCTTAACTT |
| GI29801752_PLAGL1_3R | NT_025741 | ACTCTAACGACCCATCCTAACG | AAACTTCGACTAACAAACCCCG | AAACTCTAACAACCCATCCTAACA | AAACTTCAACTAACAAACCCCAC |
| GI29803948_CD63_1R | NT_029419 | CTTACATCCTCTAAATTTCCCCG | CTTATCCCAAAAAACAAAACCAA | ACTTACATCCTCTAAATTTCCCCA | CTTATCCCAAAAAACAAAACCAA |
| GI29803948_CD63_2R | NT_029419 | ACAACTTAATCCCCCTAACCCG | TTCCAACCCTCACCTAACTTTATC | AACAACTTAATCCCCCTAACCCA | TTCCAACCCTCACCTAACTTTATC |
| GI29803948_CD63_3R | NT_029419 | ATAACGACCGATCCTACTCTCCG | CCTATTCAAATCAACTCCTTCTAAA | AATATAACAACCAATCCTACTCTCCA | CCTATTCAAATCAACTCCTTCTAAA |
| GI29803948_CDK4_1R | NT_029419 | CTAAAAACCCTTAACCCTCCG | CACAAACAAACGCTCCAAAA | TCCTAAAAACCCTTAACCCTCCA | CACAAACAAACACTCCAAAAATC |
| GI29803948_CDK4_2R | NT_029419 | ACTATTCTCTCCTATTTCCCTCG | CCCAAAATCTCCGTAAACTAAAA | AACTACTATTCTCTCCTATTTCCCTCA | CCCAAAATCTCCATAAACTAAAAA |
| GI29803948_CDK4_3R | NT_029419 | TAAAACTTTCTCCCCGAAAAACG | AAAATCCCTCTCACCCATTAACA | ATAAAACTTTCTCCCCAAAAAACA | AAAATCCCTCTCACCCATTAACA |
| GI29804485_BCAP31_1R | NT_025965 | AATTCCTCCACTTAAACTACCCG | AAAAACCGCGACCCTAAAAA | AAATTCCTCCACTTAAACTACCCA | AAAAACCACAACCCTAAAAAACC |
| GI29804485_BCAP31_2R | NT_025965 | AACTCAAAACCCTCCAAAATCG | GACCTCTTCGAACAACCCAAATA | AAACTCAAAACCCTCCAAAATCA | AACCTCTTCAAACAACCCAAATA |
| GI29804485_CTAG1_1R | NT_025965 | CTAACTAAACTCAACAACCTCCG | CCCTATCCTAATCGCCCAACTAA | CTCTAACTAAACTCAACAACCTCCA | CCCTATCCTAATCACCCAACTAA |
| GI29804485_CTAG1_2R | NT_025965 | CTTAACTAACGCGAAACCACG | CCACCAAACATACAATTCCAACT | AAAACCTTAACTAACACAAAACCACA | CCACCAAACATACAATTCCAACT |
| GI29804485_CTAG1_3R | NT_025965 | GAATCTCACTCCTAAATTCAACG | ATTCTATCCCTATCCTAATCGCC | CCAAATCTCACTCCTAAATTCAACA | ATTCTATCCCTATCCTAATCACCC |
| GI29804485_STK23_1R | NT_025965 | AAAACCTCTACCCGAAAACCG | ACTCTCTAAAACCGCCGTCG | AAAAAACCTCTACCCAAAAACCA | ACTCTCTAAAACCACCATCACACC |
| GI29804485_STK23_2R | NT_025965 | AATACGAACTACTTCACCCCCG | GTAAACCTCCCCCTCAACTAAAA | AAAATACAAACTACTTCACCCCCA | ATAAACCTCCCCCTCAACTAAAA |
| GI29804485_STK23_3R | NT_025965 | CAACCCAAAACCTACTCCTACG | CGCTACCTAACGCTCTAACTCC | CCAACCCAAAACCTACTCCTACA | CACTACCTAACACTCTAACTCCCAA |
| GI29804900_PRKCDBP_1R | NT_028310 | AAATTTAAATTACCCTCCTCCG | TCCAATTCCCTAATATACCTATTTCA | CTCTAAATTTAAATTACCCTCCTCCA | TCCAATTCCCTAATATACCTATTTCA |
| GI29804900_PRKCDBP_2R | NT_028310 | CCCCTCTAATTATCTCTTTACCG | ACCAACACAATCTCTACGCCC | AACCCCTCTAATTATCTCTTTACCA | ACCAACACAATCTCTACACCCAC |
| GI29804900_PRKCDBP_3R | NT_028310 | CAACCTATCCAAATCACAAAACG | AACCTCGAACCTTAACAATTCG | ACAACCTATCCAAATCACAAAACA | AACCTCAAACCTTAACAATTCACA |
| GI29805597_APOA1_1R | NT_033899 | CAAATCTCCCGAATAAAATCG | AATTCCCTACACTCATCCCCTT | CTTACAAATCTCCCAAATAAAATCA | AATTCCCTACACTCATCCCCTT |
| GI29805597_APOA1_2R | NT_033899 | CAAACACTCCCCTCCCG | CCCCACTAAACCCTTAACCC | ACAAACACTCCCCTCCCA | CCCCACTAAACCCTTAACCC |
| GI29805597_APOA1_3R | NT_033899 | AAACCTCAATCTAAAAACCACG | AAAACTCTCCCCTCTCCCC | CAAAACCTCAATCTAAAAACCACA | AAAACTCTCCCCTCTCCCC |
| GI29805597_PGR_1R | NT_033899 | CCCCTCCCCAAATAATCG | AAAATCCAAACTCCTTACCTCTAAT | ACCCCTCCCCAAATAATCA | AAAATCCAAACTCCTTACCTCTAAT |
| GI29805597_PGR_2R | NT_033899 | CATATTATATCCTACCATTTAATACCCG | TAAACATTCTATACCTACTTCCCAAAA | CATATTATATCCTACCATTTAATACCCA | TAAACATTCTATACCTACTTCCCAAAA |
| GI29809602_CDH13_1R | NT_024797 | AAACTCAACCTCACAAATCACG | TAAACAATACCAACTAATTCCACTTTTC | TTAAAACTCAACCTCACAAATCACA | TAAACAATACCAACTAATTCCACTTTTC |
| GI29809602_CDH13_2R | NT_024797 | CCAAAACCAATAACTTTACAAAACG | ATTCCTTCCTAACGCTCCCTC | CCAAAACCAATAACTTTACAAAACA | ATTCCTTCCTAACACTCCCTCAT |
| GI29809602_CDH13_3R | NT_024797 | AAATAAAATACCACCTCCGCG | AACTCGCTCCTCGCGAAATAC | AAACAAATAAAATACCACCTCCACA | AACTCACTCCTCACAAAATACTCAC |
| GI29809804_ASC_1R | NT_024812 | AAAAACCGCGAAAATTTCG | AATTCTAAAAATCCGAAATTCTAAACC | AATTCAAAAAACCACAAAAATTTCA | AATTCTAAAAATCCAAAATTCTAAACC |
| GI29809804_ASC_2R | NT_024812 | AAAACACCTAAACTTAAAACCTCG | ATTTCTAAAACCCCGAAACCTC | TTAAAACACCTAAACTTAAAACCTCA | ATTTCTAAAACCCCAAAACCTCC |
| GI29809804_ASC_3R | NT_024812 | AAACCAACCCAAATTTCCG | TCTATACCCGCTAATACAAACCC | ATAAAAACCAACCCAAATTTCCA | TCTATACCCACTAATACAAACCCAAA |
| GI29809804_PRSS8_1R | NT_024812 | ACCACATACACACTACACACCG | AACACACACACAAAAAACCTATACA | CACCACATACACACTACACACCA | AACACACACACAAAAAACCTATACA |
| GI29809804_PRSS8_2R | NT_024812 | ACACACACAAACCACATACACG | ACATACACGCACACTCAAACA | CACACACACAAACCACATACACA | ACATACACACACACTCAAACACAC |
| GI29809804_PRSS8_3R | NT_024812 | AAACAAACACCAACCCCG | CCCCCTTAAACCCTAAACTCA | AAAACAAACACCAACCCCA | CCCCCTTAAACCCTAAACTCA |
| GI29823171_SERPINB5_ | NT_025028 | ACATACGTACGACAATCCTCTCG | CCCACGCAAAACCGAAAA | CAAACATACATACAACAATCCTCTCA | CCCACACAAAACCAAAAAATAAA |
| 1R | | | | | |
| GI29823171_SERPINB5_ | NT_025028 | AACTCCTAAACTCAAACAATCCG | TCACGTCAACCTCCCCAAATA | CAAACTCCTAAACTCAAACAATCCA | TCACATCAACCTCCCCAAATACT |
| 3R | | | | | |
| GI29789893_CTNNB1_2R | NT_037565 | GATACTCGAAAACCGAAACCG | AATACCACCTTCCGCAAACC | AACAATACTCAAAAACCAAAACCA | AATACCACCTTCCACAAACCAC |
| GI29789893_CTNNB1_3R | NT_037565 | CACACTCCCTACTAAACCTAAATCG | AACCCAATCTCACAACACCC | CACACTCCCTACTAAACCTAAATCA | AACCCAATCTCACAACACCC |
| GI29794147_RUNX3_1R | NT_077383 | CCAACCTCAACTCACAAAATACG | AAAACCTACTCGCGACCTTAACT | CCAACCTCAACTCACAAAATACA | AAAACCTACTCACAACCTTAACTCA |
| GI29794147_RUNX3_2R | NT_077383 | TAACATCACGACCCAAATAACCG | GACCCAACCAATAAACCAAAAC | ATAACATCACAACCCAAATAACCA | AACCCAACCAATAAACCAAAACC |
| GI29794147_RUNX3_3R | NT_077383 | ACCTCGCCCCTAAACTACG | CTAACTCCCTACCATTATCCACCA | ATACCACCTCACCCCTAAACTACA | CTAACTCCCTACCATTATCCACCA |
| GI29794313_MYCL1_1R | NT_077386 | TACCAACCGACTAAACTAAACCG | ACTCCTTACCCACTCCGAACTAA | CAATACCAACCAACTAAACTAAACCA | ACTCCTTACCCACTCCAAACTAAA |
| GI29794313_MYCL1_2R | NT_077386 | AATAACCAAACGAACGCCG | AACCAACCCAAATTCAAAACTAA | CAACAAATAACCAAACAAACACCA | AACCAACCCAAATTCAAAACTAA |
| GI29799662_SCGB3A1_1R | NT_077451 | CAAAACAAAAACGAAAACGACG | AATTCCTAACTCCCTAATCCCTACC | ACCAAAACAAAAACAAAAACAACA | AATTCCTAACTCCCTAATCCCTACC |
| GI29801002_SFTPA1_1R | NT_077575 | CCAAACCAACTTATCTATCCTACG | CCTAACTCAACCATAAACCCCA | TCCAAACCAACTTATCTATCCTACA | CCTAACTCAACCATAAACCCCA |
| GI29801002_SFTPA1_2R | NT_077575 | CAAACCTAAAATTCCTCTTTCG | AAATTCTATACTCCCCTCAAAAATC | TTACAAACCTAAAATTCCTCTTTCA | AAATTCTATACTCCCCTCAAAAATC |
| GI29801002_SFTPA1_3R | NT_077575 | AACAAAAACTACAACTCTCCCG | AACCTTCCAAATACTACCCTAACTC | AAACAAAAACTACAACTCTCCCA | AACCTTCCAAATACTACCCTAACTC |
| GI29804083_UNG_1R | NT_078089 | CCAATACACTCCAAACCTAAACG | CAAAACGAAACCCTATCTCTCG | ACCAATACACTCCAAACCTAAACA | CAAAACAAAACCCTATCTCTCAA |
| GI29804083_UNG_2R | NT_078089 | AAAATTACAAACGTAAACCACCG | GCCTAACCAATCTTCTCTTCTTACA | CACTAAAATTACAAACATAAACCACCA | ACCTAACCAATCTTCTCTTCTTACAA |
| GI29804083_UNG_3R | NT_078089 | TCAAACCCTCTAACCTCAAACG | TCCTCCAACCTAAACCTCCC | ACTCAAACCCTCTAACCTCAAACA | TCCTCCAACCTAAACCTCCC |

TABLE 2
Methylation Markers for Squamous Cell Carcinoma
METHYLATION MARKERS FOR SQUAMOUS CELL CARCINOMA

| Panel_I | | Panel_II | | Panel_III | | Panel_IV | |
|---|---|---|---|---|---|---|---|
| Marker Name | p-value | Marker Name | p-value | Marker Name | p-value | Marker Name | p-value |
| HTR1B_2r | 2.559E−06 | MTHFR_1r | 0.00004 | HTR1B_2r | 0.00043 | HTR1B_2r | 0.00043 |
| GDF10_3r | 1.065E−05 | TSLL2_2r | 0.00053 | MTHFR_1r | 0.00080 | MTHFR_1r | 0.00080 |
| ARHI_2r | 2.616E−05 | HTR1B_2r | 0.00056 | MLH1_2r | 0.00099 | MLH1_2r | 0.00099 |
| SFN_1r | 4.169E−05 | ABCC5_2r | 0.00206 | GDF10_3r | 0.00183 | GDF10_3r | 0.00183 |
| CALCA_1r | 4.187E−05 | GDF10_3r | 0.00244 | APC_1r | 0.00228 | APC_1r | 0.00228 |
| ADCYAP1_2r | 4.533E−05 | MLH1_2r | 0.00290 | TP53_3r | 0.00279 | TP53_3r | 0.00279 |
| TERT_1r | 1.094E−04 | VHL_1r | 0.00350 | ARHI_2r | 0.00339 | ARHI_2r | 0.00339 |
| RASSE1_3r | 1.384E−04 | UBB_2r | 0.00400 | BRCA1_3r | 0.00397 | BRCA1_3r | 0.00397 |
| MOS_1r | 1.497E−04 | CALCA_1r | 0.00474 | CALCA_1r | 0.00468 | CALCA_1r | 0.00468 |
| SFTPC_3r | 4.626E−04 | SNCG_3r | 0.00522 | ADCYAP1_2r | 0.00505 | ADCYAP1_2r | 0.00505 |
| TERT_3r | 4.681E−04 | S100A2_1r | 0.00532 | SFTPC_3r | 0.00555 | SFTPC_3r | 0.00555 |
| MYOD1_2r | 6.924E−04 | SFN_2r | 0.00639 | TSLL2_2r | 0.00676 | TSLL2_2r | 0.00676 |
| C4B_2r | 7.712E−04 | SFTPC_3r | 0.00817 | TERT_1r | 0.00713 | TERT_1r | 0.00713 |
| CALCA_2r | 7.857E−04 | ADCYAP1_2r | 0.00910 | MYC_2r | 0.00745 | MYC_2r | 0.00745 |
| | | SFN_3r | 0.00923 | RASSF1_3r | 0.00825 | RASSF1_3r | 0.00825 |
| | | | | MOS_1r | 0.00872 | MOS_1r | 0.00872 |
| | | | | SNCG_3r | 0.00876 | SNCG_3r | 0.00876 |
| | | | | TGFBR2_3r | 0.00957 | TGFBR2_3r | 0.00957 |
| | | | | | | APC_2r | 0.01187 |
| | | | | | | HOXA5_3r | 0.01204 |
| | | | | | | TYMS_2r | 0.01442 |
| | | | | | | CDKN2B_3r | 0.01455 |
| | | | | | | CALCA_2r | 0.01579 |
| | | | | | | MYOD1_2r | 0.01594 |
| | | | | | | TERT_3r | 0.01734 |
| | | | | | | PTEN_1r | 0.01896 |
| | | | | | | C4B_2r | 0.01927 |
| | | | | | | GSTP1_3r | 0.01964 |

TABLE 3
Methylation Markers for Adenocarcinoma
Methylation Markers for Adenocarcinoma

| | |
|---|---|
| Panel_I | Panel_II |

| Marker Name | p-value | Marker Name | p-value |
|---|---|---|---|
| SFN_2r | 0.00000000007 | SFN_2r | 0.000009 |
| TWIST1_3r | 0.00000093146 | CD1A_1r | 0.000511 |
| SERPINB5_1r | 0.00000098742 | TNF_2r | 0.000526 |
| GDF10_3r | 0.00000130650 | SERPINB5_1r | 0.000638 |
| PGR_1r | 0.00000188610 | PGR_1r | 0.001117 |
| SFTPD_1r | 0.00000420738 | TWIST1_3r | 0.001288 |
| ARHI_3r | 0.00000598158 | GDF10_3r | 0.001469 |
| CALCA_1r | 0.00000933174 | SFTPD_1r | 0.002031 |
| SFTPB_1r | 0.00004835043 | ADCYAP1_2r | 0.002176 |
| CALCA_2r | 0.00008410116 | ARHI_3r | 0.002211 |
| WT1_1r | 0.00013356747 | IL13_3r | 0.002844 |
| PRDM2_3r | 0.00014989843 | HOXA5_2r | 0.002966 |
| TERT_1r | 0.00019412964 | CALCA_1r | 0.003758 |
| S100A2_2r | 0.00031181656 | HOXA5_1r | 0.004410 |
| ADCYAP1_3r | 0.00033644602 | SFTPB_1r | 0.004675 |
| RUNX3_2r | 0.00045917044 | MC2R_1r | 0.005590 |
| MYOD1_2r | 0.00061315386 | HOXA5_3r | 0.005836 |
| TWIST1_2r | 0.00077702738 | WT1_1r | 0.005920 |
| | | SERPINB5_3r | 0.008011 |
| | | CDH13_3r | 0.008365 |
| | | CALCA_2r | 0.008434 |
| | | RUNX3_1r | 0.008640 |

1. A method for identification of differentially methylated genomic CpG dinucleotide sequences associated with cancer in an individual, said method comprising:
(a) obtaining a biological sample comprising genomic DNA from said individual;
(b) measuring the level of methylated genomic CpG dinucleotide sequences for two or more of the genomic targets designated as SEQ ID NOS: 1-376 in said sample, and
(c) comparing the level of methylation at genomic CpG dinucleotide sequences in the sample to a reference level of methylated genomic CpG dinucleotide sequences, wherein a difference in the level of methylation of said genomic CpG dinucleotide sequences in the sample compared to the reference level identifies differentially methylated genomic CpG dinucleotide sequences associated with cancer.

2. The method of claim 1, wherein the level of methylation of said differentially methylated genomic CpG dinucleotide sequences is used to diagnose cancer in the individual.

3. The method of claim 1, wherein the level of methylation of said differentially methylated genomic CpG dinucleotide sequences is used to predict the course of the cancer in the individual.

4. The method of claim 1, wherein the level of methylation of said differentially methylated genomic CpG dinucleotide sequences is used to predict the susceptibility to cancer of the individual.

5. The method of claim 1, wherein the level of methylation of said differentially methylated genomic CpG dinucleotide sequences is used to stage the progression of the cancer in the individual.

6. The method of claim 1, wherein the level of methylation of said differentially methylated genomic CpG dinucleotide sequences is used to predict the likelihood of overall survival for said individual.

7. The method of claim 1, wherein the level of methylation of said differentially methylated genomic CpG dinucleotide sequences is used to predict the likelihood of recurrence of cancer for individual.

8. The method of claim 1, wherein the level of methylation of said differentially methylated genomic CpG dinucleotide sequences in said sample is used to determine the effectiveness of a treatment course undergone by the individual.

9. The method of claim 8, wherein said reference level corresponds to the level of methylated genomic CpG dinucleotide sequences present in a corresponding sample obtained from said individual prior to treatment.

10. The method of claim 1, wherein said level of methylation in the biological sample is decreased in comparison to the reference level.

11. The method of claim 1, wherein said level of methylation in the biological sample is increased in comparison to the reference level.

12. The method of claim 1, wherein said differentially methylated genomic CpG dinucleotide sequences are observed only in a subset of said genomic targets.

13. The method of claim 12, wherein said subset of targets has a methylation pattern that is characteristic of a particular type of cancer.

14. The method of claim 13, wherein said subset comprises the genomic targets set forth in Table 3 and designated SEQ ID NOS: [ ]

15. The method of claim 14, wherein said type of cancer is adenocarcenoma.

16. The method of claim 13, wherein said subset comprises the genomic targets set forth in Table 2 and designated SEQ ID NOS: [ ]

17. The method of claim 16, wherein said type of cancer is squamous cell carcenoma.

18. A population of genomic targets comprising SEQ ID NOS: 1-376.

19. A population of genomic targets selected from the group consisting of SEQ ID NOS: 1-376.

20. The population genomic markers of claim 18 or 19, wherein said targets are capable of exhibiting differential methylation of genomic CpG dinucleotide sequences, wherein said differential methylation is predictive of the presence or susceptibility of an individual for cancer.

21. The population of genomic targets of claim 19, further comprising a subset of SEQ ID NOS: 1-376.

22. The population of claim 21, wherein differential methylation of genomic CpG dinucleotide sequences in said subset is characteristic of a particular type of cancer.

23. The population of genomic targets of claim 22, wherein said subset comprises the nucleic acid sequences designated SEQ ID NOS: [ ].

24. The population of genomic targets of claim 23, wherein said type of cancer is adenocarcenoma.

25. The population of genomic targets of claim 22, wherein said subset comprises the nucleic acid sequences designated SEQ ID NOS: [ ].

26. The population of genomic targets of claim 25, wherein said type of cancer is squamous cell carcenoma.

27. A population of nucleic acid probes capable of detecting methylation of genomic CpG dinucleotide sequences of two or more genomic targets selected from the group consisting of SEQ ID NOS: 1-376.

28. The population of nucleic acid probes of claim 27, further consisting of two or more nucleic acid sequences selected from the group consisting of SEQ ID NOS: 377-1880.

29. The population nucleic acid probes of claim 27 or 28, wherein said nucleic acid probes are capable of detecting methylation of genomic CpG dinucleotide sequences of said two or more genomic targets, wherein said methylation is predictive of the presence or susceptibility of an individual for cancer.

30. The population nucleic acid probes of claim 27 or 28, wherein said nucleic acid probes are capable of detecting differential methylation of genomic CpG dinucleotide sequences of said two or more genomic targets, wherein said differential methylation is predictive of the presence or susceptibility of an individual for cancer.

31. The population of nucleic acid probes of claim 27, wherein said nucleic acid probes are capable of detecting detecting differential methylation of genomic CpG dinucleotide sequences of a subset of said two or more genomic targets.

32. The population of nucleic acid probes of claim 31, wherein differential methylation of genomic CpG dinucleotide sequences in said subset is characteristic of a particular type of cancer.

33. The population of nucleic acid probes of claim 32, wherein said nucleic acid probes comprise the nucleic acid sequences designated SEQ ID NOS:[ ].

34. The population of nucleic acid probes of claim 33, wherein said type of cancer is adenocarcinoma.

35. The population of nucleic acid probes of claim 32, wherein said nucleic acid probes comprise the nucleic acid sequences designated SEQ ID NOS:[ ].

36. The population of nucleic acid probes of claim 35, wherein said type of cancer is squamous cell carcinoma.
